(19) United States
(12) Patent Application Publication
(10) Pub. No.: US 2026/0207249 A1
(43) Pub. Date: Jul. 23, 2026

(54) SYSTEMS AND METHODS FOR TREATING JOINTS

(71) Applicant: Artezo, Inc., Morro Bay, CA (US)

(72) Inventors: Travis Jered Nolan, Carmel, CA (US); Keir David Hart, Lafayette, CO (US); Russell Delmar Hempstead, Lafayette, CO (US)

(21) Appl. No.: 19/145,045

(22) PCT Filed: Jan. 2, 2024
(86) PCT No.: PCT/US2024/010059

§ 371 Date: Jul. 1, 2025

Related U.S. Application Data
(60) Provisional application No. 63/438,432, filed on Jan. 11, 2023.

(51) Int. Cl. A61B 18/14; A61B 18/00; A61B 18/12; A61B 90/00

(52) U.S. Cl. CPC A61B 18/1477; A61B 18/1206; A61B 18/1492; A61B 90/37; A61B 2018/00434; A61B 2018/00577; A61B 2018/126; A61B 2018/1475; A61B 2090/376; A61B 2090/378

(57) ABSTRACT

Methods and systems for modulating articular branch nerves emanating from sensory and/or motor nerves to treat joint pain or facet. The methods and systems described may be used to modulate (e.g., denervate, ablate) articular branch nerves of, for example, cranial gluteal, femoral, sciatic, and/or obturator nerves, the sensory and/or motor nerves themselves, and/or other nerves innervating a joint or facet. The modulation of these nerves may facilitate treatment of chronic joint pain (e.g., hip, shoulder, knee, elbow, 1202 stifle, fetlock, carpal, etc.) and lameness (e.g., osteoarthritis (OA), degenerative joint disease (DJD), etc.). The modulation may be performed using a 1200 neuromodulation device (e.g., an energy delivery device). The device may use bipolar radiofrequency energy to form a linear or arcuate lesion to ablate a nerve. The lesion may follow curvature of bone, for example adjacent a joint capsule.

### INCORPORATION BY REFERENCE

This application claims the benefit of priority from U.S. Provisional Application No. 63/438,432 filed Jan. 11, 2023, which is incorporated in its entirety by reference, herein. Any and all priority claims identified in the Application Data Sheet, or any correction thereto, are hereby incorporated by reference under 37 CFR 1.57.

### FIELD

The present application is directed generally to medical devices, systems, and methods for treating tissue. More specifically, the application is directed to devices, systems, and methods for treating nerves, nerve fibers, or neurons to treat pain, and more particularly to therapeutically treat articular joint and/or synovial joint pain.

### DESCRIPTION OF THE RELATED ART

Osteoarthritis (OA) and degenerative joint disease (DJD) can affect both large and small animals. Non-human animals can begin developing the disease at a young age. The majority of OA in animals occur secondarily to developmental orthopedic disease, such as cranial cruciate ligament disease, hip dysplasia, elbow dysplasia, osteochondritis dissecans (OCD), patella (kneecap) dislocation, etc. In some animals, OA occurs with no obvious primary causes and can be related to genetics and age. Other contributing factors to OA can include, for example, body weight, obesity, gender, exercise, and diet.

Osteoarthritis generally begins as a disruption of the cartilage. Ultimately, this disruption causes the bones in the joint to erode into each other. Some signs or symptoms of osteoarthritis can include stiffness, lameness, and pain. The condition may start with minor pain during activity, but can develop into continuous chronic pain, which can even occur when the animal is at rest.

### SUMMARY

Joint pain can have deleterious effects such as musculoskeletal deterioration, central sensitization, and/or cognitive and/or affective decline, which can result in increased resistance to treatment. Using traditional treatment, once osteoarthritis has started in a joint, it generally cannot be fully cured and will likely affect an animal for the rest of his or her life. It is desirable to have new treatments for treating joints, in particular for treating joint pain. In several embodiments provided herein, devices and treatments are minimally invasive, reduce or negate the need for pharmaceutical remedies, and/or are permanent or at least long-lasting. In several embodiments pharmaceuticals such as pain-relieving drugs, anti-inflammatory drugs, etc. are reduced by 10-50% or more when used with the treatments described herein. The synergies between the treatments described herein and drug therapy may reduce the dose of drug, the length of drug treatment, drug tolerance, the number of doses and/or the side effects. In addition to or instead of radiofrequency, other forms of neuromodulation (e.g., ablation) may also be used (including but not limited to heat, cryo, ultrasound, microwave, etc.). Although multiple embodiments are designed for veterinary use, several of the devices described herein may be used for humans.

Modulation (e.g., denervation, ablation, etc.) of one or multiple articular nerves that branch from sensory and/or motor nerves innervating the joint, for example by radiofrequency ablation and/or other modalities, can reduce or eliminate joint pain. For example, the articular branches of the sciatic nerve, the articular branches of the cranial gluteal nerve, the articular branches of the femoral nerve, and/or the articular branches of the obturator nerve maybe ablated using a minimally invasive percutaneous treatment device. The treatment device may be configured to deliver bipolar energy between two points of the treatment device. The modulation may be performed at a location along the articular nerve, for example at or near the end of the articular nerve located in a joint capsule, at a junction between the articular branch nerve and the nerve from which it emanates, and/or locations therebetween. Without being bound by any particular theory, the modulation of the articular nerve is akin to cutting a wire so that the pain transmission signal cannot be transmitted along the cut wire (the modulated articular nerve). The modulation procedure may be minimally invasive, for example using a needle or a device advanced through a needle or catheter. Articular branches run along, or immediately adjacent, to cortical surface of bone as they approach a bone joint. Ablations can be performed where nerve fibers are bundled together to increase chances of capturing the relevant branches. However, ablation of bundled nerve fibers can be difficult due to their poor ablation profile and shape. Certain issues with standard approaches to bundled nerves is that nerves run next to critical structures (arteries, vessels, major nerves, both motor and sensory) and in most cases and it is hard to avoid collateral damage and more damage can be done to critical structures of joint (muscles, motor function, blood supply, etc.). Ablation can be performed reversibly or irreversibly. In several embodiments, denervation and/or neuromodulation may be accomplished with or without ablation.

In several embodiments, desired tissue (e.g., a nerve) is ablated while non-target tissue is preserved. In various embodiments, a treatment device beneficially achieves denervation as close to the joint as possible. Additionally, it can be difficult to know the depth of the nerves at these locations which could result in missing target nerves, resulting in poor outcomes in some cases. Thus in some embodiments, provided are devices and methods for ablating where the nerve is close to bone, which increases likelihood of capturing relevant nerve(s), provides easy anatomical reference points, and allows for the use of lower profile ablation zone (proximal/distal direction) which protects tissues/structures nearby. In one embodiment, close to the bone shall be within 1 mm to 50 mm, e.g., 1-5 mm, 5-10 mm, 10-15 mm, 15-20 mm, 20-30 mm, 30-40mm, 40-50mm, and overlapping ranges therein.

In some embodiments, ablating an area close to a joint capsule attachment or across the capsule successfully targets only, or primarily, the nerves of the joint capsule, thereby reducing vascular damage because branches to areas such as the nutrient foramen or metaphysis points have already branched and most of the blood supply to the bone capsule has already branched. In several embodiments, a treatment device is configured to fully or partially avoid ligament and tendon and their associated attachment points near the bone capsule. In some embodiments, methods of elevating a nerve ablation zone off the surface of a bone or joint involves placing the electrodes more proximal from the distal tips results in enhanced elevation of ablation off of the bone/capsule surface to avoid critical structures. For example, a device treatment position is configured to placement of a needle is on/adjacent to an important vascular structure or ligament attachment point that would avoid ablating/damaging the structure and access nerves on both sides. In one embodiment, a placement of a device, probe, and/or needle is guided by a marker. In various embodiments, an orientation device is configured to guide an angle, placement, orientation, depth, width, height of a placement of the device. In various embodiments, an orientation device is a marker. In various embodiments, the orientation device is round square, triangular, rectangular, a polyhedron, and may comprise 1, 2, 3, 4, 5, 6 or more segments. In one embodiment, a segment is a quadrant. In some embodiments, a harness, strap, arm, or other guide is used to elevate and/or orient a body party for the treatment. In various embodiments, the orientation device is scaled for size based on the specific anatomy, size, species, breed, of the patient.

In some embodiments, methods of ablating across one or more surfaces of a joint capsule involve treatment of nociceptive nerves. Sensory branches are commonly associated with the muscle groups that act on a joint and therefore, could come in a variety of locations. A benefit to this approach, in one embodiment, would be very minimal disruption to other structures, especially ligaments, tendons, arteries, veins, and nerves. Nociceptive sensory nerves are found on the surface and in the fibrous layer of joint capsules which may allow for selective targeting of nociceptive nerve endings while sparing the sympathetic nerves that are responsible for pressure, stability, motor feedback, vasoconstriction. The sympathetic nerves are in close proximity to vessels and can be found in the synovium below fibrous layers and would be protected in some embodiments.

In some embodiments, a treatment device is configured to treat one, two, three, or four quadrants of a joint. Most joints (ex. shoulder, hip, elbow) can be broken up into four general quadrants. These are broken into planes and differ by species. For example, a canine hip includes four quadrants: Cranial-lateral, Cranial-medial, Caudal-Lateral, and Caudal-Medial. In one embodiment, the treatment device is configured to create elongated ablation zones (e.g., up to 20 mm, 30 mm, 40 mm, 44 mm, 50 mm or more) with a single placement, which is designed to allow coverage and ablate the sensory nerves of an entire quadrant to simply the surgical technique and procedure. In one embodiment, the user is able to cover up to two quadrants from a single placement. In one embodiment, the user is able to cover up to three quadrants from a single placement. In one embodiment, the user is able to cover up to four quadrants from a single placement.

In some embodiments, a treatment device is configured to ablate along an extended length of a nerve to increase duration of pain relief. For example, configuring the treatment device for a thin and low profile ablation zone allows for adjustable ablation (length) along the axis of a nerve (distal-proximal in this example) while keeping collateral damage to a minimum (lateral-medial width).

In some embodiments, methods for modulating the sensory nerves of the of the dorsal spinous processes are configured to treat back pain. Kissing spine is a common problem with equines when over ridden. In various embodiments, dorsal spinous process treatment is a minimally invasive technique, performed through a small incision, used to resolve pain caused by the impingement. Dorsal spinous process treatment can be performed quicker than amputation, and convalescence after dorsal spinous process treatment is shorter than convalescence after amputation. Dorsal spinous process treatment can result in a better functional outcome than medical management. In various embodiments, dorsal spinous process treatment includes one or more of: ablating the Dorsal Ramus of the spinal nerve; Ablating the Lateral Dorsal Ramus of the spinal nerve branch; methods of sparing the medial branch of dorsal ramus to prevent multifidus muscle atrophy; methods of ablating two spinous processes with single placement of a device needle; methods of ablating long axis of medial dorsal ramus nerve branch to prolong effectiveness of pain relief; methods of modulating the lateral Dorsal Ramus n. at dorsal portion/top of spinous process where ligament attaches; and/or nerve innervating the DSP is the medial branch of the dorsal ramus n.

In various embodiments, a method of ablating an equine or canine articular nerve branch in a shoulder or hip, includes imaging an equine or canine joint capsule using at least one of ultrasound or fluoroscopy to identify anatomical landmarks in a shoulder or hip; percutaneously inserting a treatment device to a first point determined by the anatomical landmarks until a distal end of the treatment device contacts a bone, wherein the first point is disposed at one side selected from the group of: a medial side of the joint capsule and a lateral side of the joint capsule; radially extending a stylet laterally out of a needle body of the treatment device along a surface of the bone at an angle in a range of 45°-135°, wherein extending the stylet comprises sliding the stylet along the bone to a second point, wherein the needle body comprising a first electrode and the stylet comprising a second electrode; imaging the joint capsule using the at least one of ultrasound or fluoroscopy to confirm a first location of the first electrode and a second location of the second electrode relative to the anatomical landmarks; applying bipolar radiofrequency energy to the first electrode and the second electrode to ablate a first tissue between the first point and the second point, the first tissue including an articular nerve branch; retracting the stylet into the needle body; and retracting the treatment device out of the subject. In various embodiments, the stylet is extended along a length of 1-100 mm (e.g., 1-50 mm, 2-100 mm, 5-50 mm, 10-60 mm, 20-80 mm, 30-90 mm, 40-70 mm, 30-50 mm, 25-35 mm, 25-30 mm, 30-35 mm, 25-50 mm, 20-50 mm, 20-60 mm, 20-70 mm, 20-75 mm, 20-80 mm, 20-90 mm, 40-60 mm, 40-70 mm, 45-60 mm, 50-100 mm, 50-75 mm, 55-75 mm, 60-80 mm, 70-95 mm, 20-100 mm, 30-100 mm, 40-100 mm, 50-100 mm, 60-100 mm, 70-100 mm, 80-100 mm, 90-100 mm, and any values and ranges therein; e.g., 1 mm, 2 mm, 2.5 mm, 3.3 mm, 4 mm, 5 mm, 6.7 mm, 7 mm, 9 mm, 10 mm, 12 mm, 13 mm, 14 mm, 15 mm, 17 mm, 19 mm, 20 mm, 23 mm, 25 mm, 26 mm, 30 mm, 31 mm, 35 mm, 37 mm, 40 mm, 43 mm, 45 mm, 48 mm, 50 mm, 56 mm, 58 mm, 60 mm, 62 mm, 67 mm, 70 mm, 75 mm, 80 mm, 90 mm, 95 mm, 100 mm and ranges and values between such values). In various embodiments, a torque of a device such as the outer tube will be in a range of 1-6 in-lb. (e.g., 1, 1.5, 1.7, 1.9, 2, 2.1, 2.2, 2.3, 2.5, 2.8, 3, 3.3, 3.5, 3.7, 3.9, 4, 4.2, 4.5, 4.8, 5, 5.3, 5.5, 5.8 and 6 in-lb, and other ranges and values therein).

In various embodiments, a minimally invasive method of ablating an articular nerve branch adjacent to a joint capsule to treat joint pain in a subject, the method comprising: percutaneously inserting a treatment device to a first point until a distal end of the treatment device contacts a bone; radially extending a stylet laterally out of a needle body of the treatment device along a surface of the bone at an angle in a range of 45°-135°, wherein extending the stylet comprises sliding the stylet along the bone to a second point, wherein the needle body comprising a first electrode and the stylet comprising a second electrode; applying bipolar radiofrequency energy to the first electrode and the second electrode to ablate first tissue between the first point and the second point, the first tissue including an articular branch nerve; retracting the stylet into the needle body.

In one embodiment, the method includes repeating the percutaneously inserting, extending, and applying to ablate a second tissue selected from the group consisting of: a sciatic nerve, a cranial gluteal nerve, a femoral nerve, and an obturator nerve. In one embodiment, the method includes repeating the percutaneously inserting, extending, and applying to ablate a third tissue selected from the group consisting of:
the sciatic nerve, the cranial gluteal nerve, the femoral nerve, and the obturator nerve. In one embodiment, the method includes repeating the percutaneously inserting, extending, and applying to ablate a fourth tissue selected from the group consisting of: the sciatic nerve, the cranial gluteal nerve, the femoral nerve, and the obturator nerve. In one embodiment, the subject is a quadruped. In one embodiment, the joint capsule is in a hip, a shoulder, a knee, an elbow, a stifle, a fetlock, or a carpal. In one embodiment, the method includes generating coordinates for a target treatment site as a function of a calculated distance from the anatomical landmarks. In one embodiment, the subject has osteoarthritis or degenerative joint disease. In various embodiments, an angle in a range (fixed or variable) of 20-160 degrees (e.g., 30°-140°, 45°-135°, 60°-120°, 80°-110°, 20°, 25°, 30°, 35°, 40°, 45°, 50°, 55°, 60°, 65°, 70°, 75°, 80°, 85°, 90°, 95°, 100°, 110°, 115°, 120°, 125°, 130°, 135°, 140°, 145°, 150°, 155°, 160°, and any range or value of angles therein).

In various embodiments, a minimally invasive method of ablating an articular nerve branch adjacent a joint quadrant to treat joint pain in a subject, the method comprising: identifying an anatomical landmark; percutaneously inserting a treatment device to a first point determined by the anatomical landmarks until a distal end of the treatment device contacts a bone, radially extending a stylet laterally out of a needle body of the treatment device along a surface of the bone at an angle in a range of 45°-135°, wherein extending the stylet comprises sliding the stylet along the bone to a second point, wherein the needle body comprising a first electrode and the stylet comprising a second electrode; wherein a first region between the first point and the second point corresponds to a first joint quadrant; applying bipolar radiofrequency energy to the first electrode and the second electrode to ablate a first tissue between the first point and the second point, the first tissue including an articular nerve branch; retracting the stylet into the needle body; and retracting the treatment device out of the subject.

In one embodiment, extending the stylet further comprises: sliding the stylet along the bone from the second point to a third point, wherein a second region between the second point and the third point corresponds to a second joint quadrant. In one embodiment, extending the stylet further comprises: sliding the stylet along the bone from the second point to a third point, wherein a second region between the second point and the third point corresponds to a third joint quadrant. In one embodiment, extending the stylet further comprises: sliding the stylet along the bone from the second point to a third point, wherein a second region between the second point and the third point corresponds to a fourth joint quadrant. In one embodiment, the subject is a quadruped. In one embodiment, the first joint quadrant is in a hip, a shoulder, a knee, an elbow, a stifle, a fetlock, or a carpal. In one embodiment, the method includes generating coordinates for a target treatment site as a function of a calculated distance from the anatomical landmarks. In one embodiment, the subject has osteoarthritis or degenerative joint disease.

In various embodiments, a minimally invasive method of ablating an extended length of an articular nerve to increase a duration of joint pain relief, the method comprising: percutaneously inserting a treatment device to a first point determined by an anatomical landmark; radially extending a stylet laterally out of a needle body of the treatment device along a surface of a bone at an angle in a range of 45°-135°, wherein extending the stylet comprises sliding the stylet along the bone to a second point lengthwise along an articular nerve, wherein the needle body comprising a first electrode and the stylet comprising a second electrode; imaging using at least one of ultrasound or fluoroscopy to confirm a location of the first electrode and the second electrode relative to the anatomical landmarks; applying bipolar radiofrequency energy to the first electrode and the second electrode to ablate nerve tissue along a length of the nerve between the first point and the second point; retracting the stylet into the needle body; and retracting the treatment device out of the subject. In one embodiment, the length of the nerve between the first point and the second point comprises a plurality of nociceptors.

In various embodiments, a minimally invasive method of modulating the sensory nerves of the dorsal spinous processes in a subject, the method comprising: percutaneously inserting a treatment device to a first point determined by the anatomical landmarks, wherein the first point is disposed at a first spinous process of a first vertebrae of the subject; radially extending a stylet laterally out of a needle body of the treatment device along a surface of the first vertebrae at an angle in a range of 45°-135°, wherein extending the stylet comprises sliding the stylet along the bone to a second point, wherein the needle body comprising a first electrode and the stylet comprising a second electrode; imaging using at least one of ultrasound or fluoroscopy to confirm a location of the first electrode and the second electrode relative to the anatomical landmarks; applying bipolar radiofrequency energy to the first electrode and the second electrode to ablate a first tissue between the first point and the second point; retracting the stylet into the needle body; and retracting the treatment device out of the subject.

In one embodiment, the first tissue is one or more of: a dorsal ramus of the first vertebrae, a lateral dorsal ramus of the first vertebrae, a medial dorsal ramus of the first vertebrae. In one embodiment, the second point is one of: a transverse process of the first vertebrae or a transverse process of a second vertebrae.

In various embodiments, a minimally invasive device for treating tissue, the device comprising: a needle body comprising: a tissue penetrating tip; and an aperture in a side of the needle body; a stylet configured to radially extend from the aperture of the needle body at an angle in a range of 45°-135°, wherein the stylet is configured for extending along a surface of a bone, the stylet comprising: a first electrode, and a tissue penetrating tip; a second electrode disposed along the needle body between the tissue penetrating tip of the needle body and the stylet; and a handle configured to be coupled to a tissue treatment system, the handle comprising a stylet mechanism configured to longitudinally move the stylet relative to the needle body.

In one embodiment, the needle body comprises a needle body temperature sensor. In one embodiment, the stylet comprises a stylet temperature sensor. In one embodiment, the stylet is configured to radially extend from the aperture of the needle body along a curved path. In one embodiment, the stylet mechanism comprises a knob configured to slide along a path. In one embodiment, the stylet mechanism comprises the handle being rotatable relative to the needle body. In one embodiment, the stylet mechanism comprises a rotatable knob. In one embodiment, the needle body comprises the first electrode. In one embodiment, the tissue penetrating tip of the needle body comprises the first electrode. In one embodiment, the tissue penetrating tip of the stylet comprises the first electrode. In one embodiment, the tissue penetrating tip of the stylet comprises the first electrode. In one embodiment, the stylet mechanism comprises indicia configured to inform a user about an extend of longitudinal movement of the stylet. In one embodiment, the stylet mechanism comprises detents configured to inform a user about an extend of longitudinal movement of the stylet. In one embodiment, the device includes electronic circuitry configured to track use information. In one embodiment, the device includes a longitudinally movable ramp, wherein a longitudinal position of the ramp affects a longitudinal position at which the stylet radially extends from the aperture of the needle body. In one embodiment, the handle comprises a ramp mechanism configured to longitudinally move the ramp relative to the needle body. In one embodiment, the stylet is steerable. In one embodiment, the stylet is steerable in one direction. In one embodiment, the stylet is steerable in two directions. In one embodiment, the two directions are on one plane. In one embodiment, the stylet comprises one steering wire. In one embodiment, the stylet comprises two steering wires. In one embodiment, the stylet comprises a tube. In one embodiment, the tube comprises a first plurality of kerfs on a first side of the tube and a second plurality of kerfs on a second side of the tube opposite the first side. In one embodiment, at least one of a shape, size, or spacing of the first plurality of kerfs is different than at least one of a shape, size, or spacing of the second plurality of kerfs. In one embodiment, a distal portion of the tube comprises a pattern and material bent radially inwardly to attach a steering wire between the material and an inner surface of the tube. In one embodiment, the stylet mechanism comprises a knob configured to slide along a path and to rotate relative to the needle body, wherein rotation of the knob is configured to steer the stylet. In one embodiment, the stylet comprises the first electrode and the second electrode longitudinally movable relative to each other. In one embodiment, the stylet comprises a first tube comprising the first electrode and a second tube comprising a second electrode, the second tube in telescoping arrangement with the first tube. In one embodiment, the stylet comprises a tube comprising a shape memory material.

In various embodiments, a method of ablating an equine or canine articular nerve branch in the method comprising: imaging a joint capsule using at least one of ultrasound or fluoroscopy to identify anatomical landmarks; percutaneously inserting a treatment device to a first point determined by the anatomical landmarks until a distal end of the treatment device contacts a bone, wherein the first point is disposed at one side selected from the group of: a medial side of the joint capsule and a lateral side of the joint capsule; radially extending a stylet laterally out of a needle body of the treatment device at an angle in a range of 45°-135°, wherein extending the stylet comprises sliding the stylet along the bone to a second point, wherein the needle body comprising a first electrode and the stylet comprising a second electrode; imaging the joint capsule using the at least one of ultrasound or fluoroscopy to confirm a first location of the first electrode and a second location of the second electrode relative to the anatomical landmarks; applying bipolar radiofrequency energy to the first electrode and the second electrode to ablate a first tissue between the first point and the second point, the first tissue including an articular nerve branch; retracting the stylet into the needle body; and retracting the treatment device out of the subject.

In various embodiments, a minimally invasive method of ablating an articular nerve branch adjacent to a joint capsule to treat joint pain in a subject, the method comprising: percutaneously inserting a treatment device to a first point until a distal end of the treatment device contacts a bone; radially extending a stylet laterally out of a needle body of the treatment device at an angle in a range of 45°-135°, wherein extending the stylet comprises sliding the stylet along the bone to a second point, wherein the needle body comprising a first electrode and the stylet comprising a second electrode; applying bipolar radiofrequency energy to the first electrode and the second electrode to ablate first tissue between the first point and the second point, the first tissue including an articular branch nerve; retracting the stylet into the needle body.

In some embodiments, a minimally invasive method of modulating an articular nerve branch to treat hip joint pain in a subject comprises imaging the hip joint using at least one of ultrasound or fluoroscopy to identify anatomical landmarks, percutaneously inserting a treatment device to a first point determined by the anatomical landmarks until a distal end of the treatment device contacts bone, and extending a stylet out of a needle body of the treatment device. Extending the stylet comprises sliding the stylet along the bone to a second point. The needle body comprises a first electrode, and the stylet comprises a second electrode. The method comprises imaging using at least one of ultrasound or fluoroscopy to confirm a first location of the first electrode and the second electrode relative to the anatomical landmarks, and applying bipolar radiofrequency energy to the first electrode and the second electrode to ablate first tissue between the first point and the second point. The first tissue includes an articular branch nerve of a sciatic nerve. The method comprises retracting the stylet into the needle body, rotating the treatment device at the first point, and extending the stylet out of the needle body of the treatment device. Extending the stylet comprises sliding the stylet along the bone to a third point. The method comprises imaging using at least one of ultrasound or fluoroscopy to confirm a second location of the first electrode and the second electrode relative to the anatomical landmarks, and applying bipolar radiofrequency energy to the first electrode and the second electrode to ablate second tissue between the first point and the third point. The second tissue includes an articular branch nerve of a cranial gluteal nerve. The method comprises retracting the stylet into the needle body, and retracting the treatment device out of the subject.

In some embodiments, a minimally invasive method of modulating an articular nerve branch to treat hip joint pain in a subject comprises imaging the hip joint using at least one of ultrasound or fluoroscopy to identify anatomical landmarks, percutaneously inserting a treatment device to a first point determined by the anatomical landmarks until a distal end of the treatment device contacts bone, and extending a stylet out of a needle body of the treatment device. Extending the stylet comprises sliding the stylet along the bone to a second point. The needle body comprises a first electrode and the stylet comprising a second electrode. The method comprises imaging using at least one of ultrasound or fluoroscopy to confirm a first location of the first electrode and the second electrode relative to the anatomical landmarks, and applying bipolar radiofrequency energy to the first electrode and the second electrode to ablate first tissue between the first point and the second point. The first tissue includes an articular branch nerve of a sciatic nerve. The method comprises retracting the stylet into the needle body, retracting the treatment device out of the subject, percutaneously inserting the treatment device to a third point determined by the anatomical landmarks until the distal end of the treatment device contacts the bone, and extending the stylet out of the needle body of the treatment device. Extending the stylet comprises sliding the stylet along the bone to a fourth point. The method comprises imaging using at least one of ultrasound or fluoroscopy to confirm a second location of the first electrode and the second electrode relative to the anatomical landmarks, and applying bipolar radiofrequency energy to the first electrode and the second electrode to ablate second tissue between the third point and the fourth point. The second tissue includes an articular branch nerve of a cranial gluteal nerve. The method comprises retracting the stylet into the needle body, and retracting the treatment device out of the subject.

The above methods and other methods described herein may comprise percutaneously inserting the treatment device to a fifth point determined by the anatomical landmarks until a distal end of the treatment device contacts bone, and extending the stylet out of the needle body of the treatment device. Extending the stylet may comprise sliding the stylet along the bone to a sixth point. The method may comprise imaging using at least one of ultrasound or fluoroscopy to confirm a third location of the first electrode and the second electrode relative to the anatomical landmarks. The method may comprise applying bipolar radiofrequency energy to the first electrode and the second electrode to ablate third tissue between the fifth point and the sixth point. The third tissue may include an articular branch nerve of a femoral nerve. The method may comprise retracting the stylet into the needle body, and retracting the treatment device out of the subject.

The above methods or other methods described herein may comprise percutaneously inserting the treatment device to a seventh point determined by the anatomical landmarks until a distal end of the treatment device contacts bone, and extending the stylet out of the needle body of the treatment device. Extending the stylet may comprise sliding the stylet along the bone to an eighth point. The method may comprise imaging using at least one of ultrasound or fluoroscopy to confirm a fourth location of the first electrode and the second electrode relative to the anatomical landmarks. The method may comprise applying bipolar radiofrequency energy to the first electrode and the second electrode to ablate fourth tissue between the seventh point and the eighth point. The fourth tissue may include an articular branch nerve of an obturator nerve. The method may comprise retracting the stylet into the needle body, and retracting the treatment device out of the subject.

In some embodiments, a method of modulating an articular nerve branch to treat hip joint pain in a subject comprises percutaneously inserting a treatment device to a first point until a distal end of the treatment device contacts bone, and extending a stylet out of a needle body of the treatment device. Extending the stylet comprises sliding the stylet along the bone to a second point. The needle body comprises a first electrode and the stylet comprising a second electrode. The method comprises applying bipolar radiofrequency energy to the first electrode and the second electrode to ablate first tissue between the first point and the second point. The first tissue includes an articular branch nerve of a first one of a sciatic nerve, a cranial gluteal nerve, a femoral nerve, or an obturator nerve. The method comprises retracting the stylet into the needle body, and repeating the percutaneously inserting, extending, and applying to ablate second tissue including an articular branch nerve of a second one of the sciatic nerve, the cranial gluteal nerve, the femoral nerve, or the obturator nerve.

The first one may be the articular branch of the sciatic nerve and the second one may be the articular branch of the cranial gluteal nerve. The first one may be the articular branch of the cranial gluteal nerve and the second one may be the articular branch of the sciatic nerve. The method may comprise repeating the percutaneously inserting, extending, and applying to ablate second tissue including an articular branch nerve of a third one of the sciatic nerve, the cranial gluteal nerve, the femoral nerve, or the obturator nerve. The method may comprise repeating the percutaneously inserting, extending, and applying to ablate second tissue including an articular branch nerve of a fourth one of the sciatic nerve, the cranial gluteal nerve, the femoral nerve, or the obturator nerve.

In some embodiments, a minimally invasive method of modulating an articular nerve branch to treat pain in a joint of a subject comprises identifying an articular branch nerve emanating from a nerve innervating the joint (e.g., a sensory and/or motor nerve), and percutaneously modulating the articular branch nerve. After modulating the articular branch nerve, the pain is reduced.

The joint may comprise a hip. Identifying the articular branch nerve may comprise acquiring a hip joint reference point corresponding to a radiographically identifiable anatomical feature of the hip joint, and generating coordinates for a target treatment site as a function of a calculated distance from the reference point. The nerve may comprise at least one of the cranial gluteal nerve, femoral nerve, sciatic nerve, or obturator nerve. Modulating the articular branch nerve may comprise modulating the articular branch nerve at a target site. The target site may be external to the joint. The target site may comprise a portion of the articular branch nerve proximate a junction between the articular branch nerve and the nerve, a portion of the articular branch nerve proximate a terminus of the articular branch nerve, a portion of the articular branch nerve between a junction between the articular branch nerve and the nerve and a terminus of the articular branch nerve, and/or combinations thereof. Modulating the articular branch nerve may comprise percutaneously inserting a treatment device to a first point, extending a stylet out of a needle body of the treatment device, the needle body comprising a first electrode and the stylet comprising a second electrode, and applying energy to the treatment device to ablate tissue including the articular branch nerve. Applying the energy may comprise applying radiofrequency energy. The radiofrequency energy may be bipolar. The method may further comprise modulating the nerve.

In the above methods or other methods described herein, the subject may be a quadruped. The subject may be a canine. The subject may have osteoarthritis or degenerative joint disease.

In some embodiments, a device for treating tissue comprises, or alternatively consists essentially of, a needle body and a stylet configured to radially extend from the aperture of the needle body along a curved path, and a handle. The needle body comprises a first electrode, a tissue penetrating tip, a first temperature sensor, and an aperture in a side of the needle body. The stylet comprises a second electrode, a tissue penetrating tip, and a second temperature sensor. The handle is configured to be coupled to a tissue treatment system. The handle comprises a stylet mechanism configured to longitudinally move the stylet relative to the needle body.

The needle body may comprise a radiopaque material. The stylet may comprise a radiopaque material. The needle body may comprise an echogenic surface. The stylet may comprise an echogenic surface. The needle body may comprise stainless steel. The stylet may comprise a shape memory material. The shape memory material may comprise nitinol. The stylet mechanism may comprise a knob configured to slide along a path. The stylet mechanism may comprise the handle being rotatable relative to the needle body. The stylet mechanism may comprise a rotatable knob. The stylet mechanism may comprise indicia configured to inform a user about an extend of longitudinal movement of the stylet. The stylet mechanism may comprise detents configured to inform a user about an extend of longitudinal movement of the stylet. The device may comprise electronic circuitry configured to track use information. A kit may comprise the device and a guide catheter. The guide catheter may comprise a first lumen configured to accommodate passage of an imager, and a second lumen configured to accommodate passage of the device. A kit may comprise the device, and a tissue treatment system.

The tissue treatment system may comprise a display screen, an energy generator, a control computer, and a removable connector configured to couple the device and the system. The removable connector may comprise electronic circuitry configured to track use information. The tissue treatment system may comprise an imaging device.

In some embodiments, a device for treating tissue comprises, or alternatively consists essentially of, a needle body comprising a tissue penetrating tip and an aperture in a side of the needle body. The device comprises a first electrode and a stylet configured to radially extend from the aperture of the needle body. The stylet comprises a second electrode and a tissue penetrating tip. The device comprises a handle configured to be coupled to a tissue treatment system. The handle comprises a stylet mechanism configured to longitudinally move the stylet relative to the needle body.

The needle body may comprise a first temperature sensor. The stylet may comprise a second temperature sensor. The stylet may be configured to radially extend from the aperture of the needle body along a curved path. The stylet mechanism may comprise a knob configured to slide along a path. The stylet mechanism may comprise the handle being rotatable relative to the needle body. The stylet mechanism may comprise a rotatable knob. The needle body may comprise the first electrode. The tissue penetrating tip of the needle body may comprise the first electrode. The stylet may comprise the first electrode. The tissue penetrating tip of the stylet may comprise the first electrode. The stylet mechanism may comprise indicia configured to inform a user about an extend of longitudinal movement of the stylet. The stylet mechanism may comprise detents configured to inform a user about an extend of longitudinal movement of the stylet. The device may comprise electronic circuitry configured to track use information. The device may comprise a longitudinally movable ramp. The longitudinal position of the ramp may affect a longitudinal position at which the stylet radially extends from the aperture of the needle body. The handle may comprise a ramp mechanism configured to longitudinally move the ramp relative to the needle body. The stylet may be steerable. The stylet may be steerable in one direction. The stylet may be steerable in two directions. The two directions may be on one plane. The stylet may comprise one steering wire. The stylet may comprise two steering wires. The stylet may comprise a tube. The tube may comprise a first plurality of kerfs on a first side of the tube and a second plurality of kerfs on a second side of the tube opposite the first side. At least one of a shape, size, or spacing of the first plurality of kerfs may be different than at least one of a shape, size, or spacing of the second plurality of kerfs. A distal portion of the tube may comprise a pattern and material bent radially inwardly to attach a steering wire between the material and an inner surface of the tube. The stylet mechanism may comprise a knob configured to slide along a path and to rotate relative to the needle body. Rotation of the knob may be configured to steer the stylet. The stylet may comprise the first electrode and the second electrode longitudinally movable relative to each other. The stylet may comprise a first tube comprising the first electrode and a second tube comprising a second electrode. The second tube may be in telescoping arrangement with the first tube. The may comprise a tube comprising a shape memory material.

In some embodiments, a minimally invasive method of treating pain in an arcuate joint capsule of a subject comprises percutaneously positioning a treatment device at a first point along the capsule extending a stylet to a second point along the capsule, applying energy to the device to create a first lesion along the capsule, retracting the stylet, extending the stylet to a third point along the capsule, and applying energy to the device to create a second lesion along the capsule. The second lesion is at an angle to the first lesion.

The first lesion may have a thickness to length ratio between 1.25:1 and 10:1. The second lesion may have a thickness to length ratio between 1.25:1 and 10:1. The capsule may be a hip joint capsule. The subject may be a quadruped.

In some embodiments, a minimally invasive method of treating pain in a subject comprises positioning a treatment device at a first point and extending a stylet from the treatment device to a second point. After extending the stylet to the second point a first electrode is proximate to or touching a bone of the subject and the stylet comprises a second electrode proximate to or touching the bone. The method further comprises applying energy to the device to create a lesion along the bone. The lesion follows the curvature of the bone.

Applying the energy may comprise applying bipolar radiofrequency energy. The bone may include an arcuate rim. The treatment device may comprise the first electrode. The stylet may comprise the first electrode. The bone may be part of a hip joint capsule. The subject may be a quadruped.

In some embodiments, a treatment system comprises, consists essentially of, or consists of one or more of the features described herein.

In some embodiments, a tissue treatment system comprises, consists essentially of, or consists of one or more of the features described herein.

In some embodiments, a method of modulating an articulator nerve comprises, consists essentially of, or consists of one or more of the features described herein. In several embodiments, treatment systems and methods are configured to operate with systems and methods disclosure in International App. PCT/US2022/073450 filed on Jul. 6, 2022 and U.S. App. 63/218,676 filed on Jul. 6, 2021, each of which is hereby incorporated by reference in its entirety, herein.

### BRIEF DESCRIPTION OF THE DRAWINGS

The following drawings are for illustrative purposes only and show non-limiting embodiments. Features from different figures may be combined in several embodiments.

FIG. 1A is a topographic illustration showing the innervation of the lateral canine hip joint.

FIG. 1B is a topographic illustration showing the innervation of the ventral canine hip joint.

FIG. 2 is a schematic drawing revealing articular branches of sciatic nerve supplying the dorsal portion of hip joint capsule.

FIG. 3 is an illustration displaying innervation of the craniolateral part of the hip joint capsule by the branches from the cranial gluteal nerve.

FIG. 4 is a diagram showing one of the articular branches of the femoral nerve innervating the cranioventral part of the hip joint capsule by passing through the m. iliopsoas.

FIG. 5 is a schematic drawing presenting the articular branch of the obturator nerve.

FIG. 6A schematically illustrates an example treatment device.

FIG. 6B schematically illustrates an example delivery device for a treatment device.

FIG. 7 is a schematic diagram of an example tissue treatment system.

FIGS. 8A and 8B illustrate an example method of treating hip joint pain.

FIG. 9A is a partial cross-sectional perspective view of an example tissue treatment device.

FIG. 9B is a partial perspective view of an example component of the tissue treatment device of FIG. 9A.

FIG. 9C is a partial cross-sectional side view of the tissue treatment device of FIG. 9A.

FIG. 9D is a schematic diagram of the tissue treatment device of FIG. 9A.

FIG. 9E is another schematic diagram of the tissue treatment device of FIG. 9A.

FIG. 10A is a partial perspective view of an example tissue treatment device.

FIG. 10B is a partial perspective view of the tissue treatment device of FIG. 10A with a component removed.

FIG. 10C is a partial cross-sectional distal end view of an example component of the tissue treatment device of FIG. 10A.

FIG. 10D is a partial perspective view of the tissue treatment device of FIG. 10A with additional components removed.

FIG. 10E is a partial cross-sectional side view of the tissue treatment device of FIG. 10A along the line 10E-10E.

FIG. 10F is a schematic diagram of the tissue treatment device of FIG. 10A.

FIG. 10G is another schematic diagram of the tissue treatment device of FIG. 10A.

FIG. 11A is a partial perspective view of an example tissue treatment device.

FIG. 11B is a partial perspective view of the tissue treatment device of FIG. 11A with a component removed.

FIG. 11Ci is a side view of an example component of the tissue treatment device of FIG. 11A.

FIG. 11Cii is an expanded side view of the example component of FIG. 11Ci.

FIG. 11D is a partial perspective view of the tissue treatment device of FIG. 11A with another component removed.

FIG. 11E is a partial cross-sectional side view of the tissue treatment device of FIG. 11A along the line 11E-11E.

FIG. 11Fi is a partial side and distal end perspective view of example components of the tissue treatment device of FIG. 11A.

FIG. 11Fii is a partial distal end view of the example components of FIG. 11Ei.

FIG. 12A is a partial perspective view of an example tissue treatment device.

FIG. 12B is a partial cross-sectional side view of the tissue treatment device of FIG. 12A along the line 12B-12B.

FIG. 12C is a schematic diagram of the tissue treatment device of FIG. 12A.

FIG. 12D is another schematic diagram of the tissue treatment device of FIG. 12A.

FIG. 12E is partial side view of an example tissue treatment device.

FIG. 12F is partial top view of the tissue treatment device of FIG. 12E.

FIG. 12G is another partial side view the tissue treatment device of FIG. 12E.

FIG. 12H is partial bottom view of the tissue treatment device of FIG. 12E.

FIG. 12I is a side and detail view of a 5 mm deployment of the tissue treatment device of FIG. 12E.

FIG. 12J is a side and detail view of a 20 mm deployment of the tissue treatment device of FIG. 12E.

FIG. 12K is a side and detail view of a 22 mm deployment of the tissue treatment device of FIG. 12E.

FIG. 12L is an isometric view of the tissue treatment device of FIG. 12E.

FIGS. 12M-12N are schematic views of cleaning a retracted tissue treatment device of FIG. 12E.

FIG. 12O-12P are schematic views of cleaning an extended tissue treatment device of FIG. 12E. FIGS. 13A-13D are schematic depictions of example lesions.

FIGS. 13E-13F are other schematic depictions of example lesions.

FIG. 14 is a schematic front (left) and side (right) depiction of example lesion.

FIG. 15A is an example method of treating hip joint pain.

FIG. 15B is another example method of treating hip joint pain.

FIG. 15C is another example method of treating hip joint pain in multiple quadrants.

FIG. 15D is another example method of treating hip joint pain in multiple quadrants.

FIG. 16A illustrates an example of placement of a probe via a lateral view of a knee joint.

FIG. 16B illustrates an example of placement of a probe via an anterior view of a knee joint.

FIG. 16C illustrates an example of placement of a probe via a medial view of a knee joint.

FIG. 17A is a schematic of an example treatment device.

FIG. 17B illustrates how the treatment device may be used to treat a subject without damaging a vessel.

FIG. 18 illustrates an example placement of a treatment device along the length of a nerve.

FIG. 19 illustrates a method for treating pain associated with a spinous process

FIG. 20A illustrates an example of placement of a probe via an anterior view of an equine stifle.

FIG. 20B illustrates an example of placement of a probe via a medial view of an equine stifle.

FIG. 20C illustrates an example of placement of a probe via a lateral view of an equine stifle.

FIG. 21A illustrates an example of placement of a probe via a medial view of a canine knee.

FIG. 21B illustrates an example of placement of a probe via a lateral view of a canine knee.

FIG. 22A illustrates an example of placement of a probe via a cranial view of an elbow.

FIG. 22B illustrates an example of placement of a probe via a lateral view of an elbow.

FIG. 22C illustrates an example of placement of a probe via a medial view of an elbow.

FIG. 23A illustrates an example of placement of a probe via a lateral view of a shoulder.

FIG. 23B illustrates an example of placement of a probe via a lateral view of a shoulder.

FIG. 23C illustrates an example of placement of a probe via a lateral view of a shoulder.

FIG. 24A illustrates an example of placement of a probe via a medial view of a metacarpophalangeal joint.

FIG. 24B illustrates an example of placement of a probe via a lateral view of a metacarpophalangeal joint.

FIG. 25A illustrates an example of placement of a probe via a medial view of a metatarsophalangeal joint.

FIG. 25B illustrates an example of placement of a probe via a lateral view of a metatarsophalangeal joint.

FIG. 25C illustrates an example incision for placement of a probe via a cranial view of a metatarsophalangeal joint.

FIG. 26A illustrates an example of placement of a probe via a medial view of a hock.

FIG. 26B illustrates an example of placement of a probe via a lateral view of a hock.

FIG. 27 illustrates an example of placement of a probe via a lateral view of a coffin joint.

### DETAILED DESCRIPTION

Joint pain that can be due to a variety of problems in both humans and non-human animals (e.g., canine, equine, feline, porcine, pachyderm, etc.) can be debilitating. Treatments for joint pain vary widely, and can include physical therapy, pharmacological therapy, surgical intervention, and/or others. Use of pharmacological therapy to treat joint pain can present several concerns. First, long term use can carry a high risk of complications to the animal's gastrointestinal tract, kidney, liver, and/or other organs. Second, the cost of the pharmacological therapy, usually over several years, may be extremely costly. Third, the pain generally persists for many years. Surgery can also present several concerns. First, most techniques disadvantageously involve removing part or most of the joint (osteotomy), surgically removing or transecting the nerves, and/or implanting a prosthesis. While surgery may provide long-term relief, surgical techniques have the disadvantage of being extremely expensive, having extensive recovery time, and being fraught with high complications rates (up to 20%). Additionally, many subjects (e.g., old, obese, etc.) may be unable to undergo surgery. While physical therapy does not necessarily present all the concerns of surgery or using pharmacological therapies, subjects receive varying degrees of pain relief, ranging from none or minimal to total. Additionally, physical therapy may provide only short-term pain relief, thereby extending treatment over several years, and thus increasing the cost of treatment. Moreover, many subjects ultimately require surgical intervention. Pharmaceuticals, surgery, and/or physical therapy may be combined with other treatments described herein, for example but not limited to, use in a manner that may reduce or eliminate one, some, or all the concerns about such treatment. In several embodiments pharmaceuticals such as pain-relieving drugs, anti-inflammatory drugs, etc. are reduced by 10-50% or more when used with the treatments described herein. The synergies between the treatments described herein and drug therapy may reduce the dose of drug, the length of drug treatment, drug tolerance, the number of doses and/or the side effects.

Minimally invasive techniques for accessing the nerves have been developed through the surgical techniques used in regional nerve blocks, intra-articular joint injections, and articular denervation (e.g., ablation, neurectomy, etc.). Some procedures useful in humans do not translate well to other, non-human animal species (e.g., canine, equine, feline, porcine, pachyderm, etc.). Clear anatomical differences exist (e.g., different joints, nerves, access routes, sizes, etc.), but even how nerves innervate the joints is different. Different minimally invasive techniques of accessing the nerves are preferably used when treating non-human animals such as quadrupeds. In these species, minimally invasive techniques of accessing the nerves through surgical techniques are useful in regional nerve blocks, intra-articular joint injections, and surgical joint capsule denervation (e.g., transection of articular nerve branches by surgically removing the periosteum).

The sensory and/or motor nerves have small nerve branches, known as articular nerves or articular nerve branches, that emanate from the trunk of the nerve and enter into the joint capsule or terminate in muscles adjacent to the joint capsule. Modulation of one articular nerve or a plurality of articular nerves can reduce or eliminate pain at that joint. The modulation may be performed at a location along the articular nerve at or near the end located in the joint capsule, for example to avoid modulating other anatomy, but modulation anywhere along the articular nerve may be effective. The modulation may be performed at a location along the articular nerve, for example at or near the end of the articular nerve located in a joint capsule, at a junction between the articular branch nerve and the nerve from which it emanates, and/or locations therebetween.

Hip joint arthritis (HJA) is one of the most common orthopedic problems in both humans and non-human animals (e.g., canine, equine, feline, porcine, pachyderm, etc.). Hip joint arthritis is usually accompanied by severe pain originating mainly from the richly innervated hip joint capsule. In canines, hip dysplasia is a common genetic disorder in which the ball and socket of the hip joint do not fit properly, causing rubbing and grinding instead of smooth sliding. A treatment based on modulation (e.g., inhibition, denervation, ablation, etc.) of articular nerves in and/or around the joint capsule may be a cost-effective alternative method for reduction of pain. For the subject or patient, this type of treatment can provide a significant improvement in the quality of life and, more importantly, significant slowing of the atrophy of pelvic limb muscles. Modulation (e.g., stimulation) of one or more nerves identified herein is provided in several embodiments. In some embodiments, reduction of neurotransmitter activity or release and/or increased neurotransmitter uptake or degradation is accomplished. In some embodiments, increased neurotransmitter activity or release and/or reduction of neurotransmitter uptake or degradation is accomplished. Examples of neurotransmitters include GABA, substance P, glutamate, dopamine, etc. In some embodiments, nociceptors are modulated. In several embodiments, the neuromodulation techniques described herein may be used synergistically with drugs or to replace drugs. This may advantageously reduce undesired side effects from drugs, dependence, tolerance, withdrawal, etc. In one embodiment, neuromodulation can reduce the dose, amount, time etc. of the drug required to achieve relief.

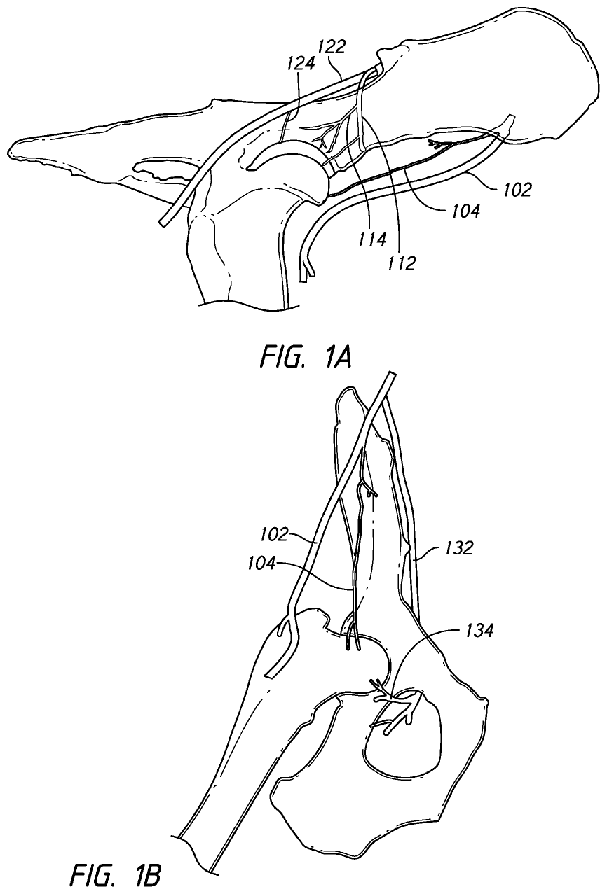

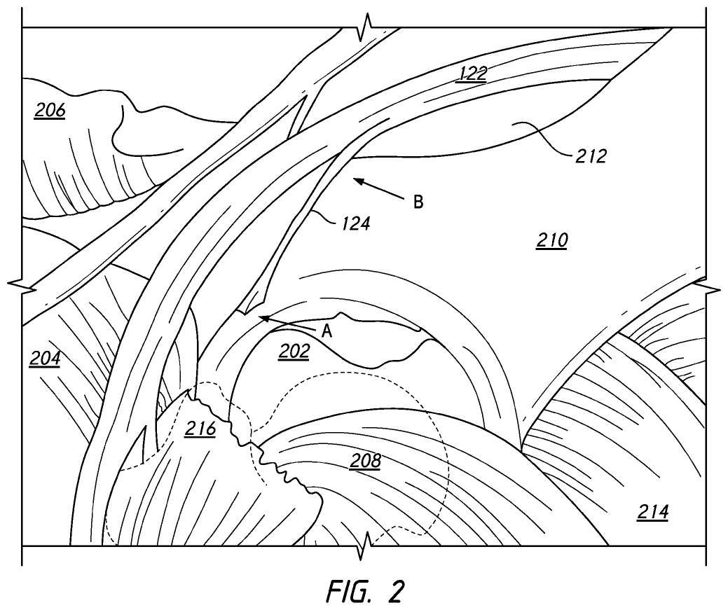

FIG. 1A is a topographic illustration showing the innervation of the lateral canine hip joint. FIG. 1B is a topographic illustration showing the innervation of the ventral canine hip joint. FIG. 1A shows a femoral nerve 102, articular branches 104 from femoral nerve 102, a cranial gluteal nerve 112, articular branches 114 from the cranial gluteal nerve 112, a sciatic nerve 122, and articular branches 124 from the sciatic nerve 122. FIG. 1B shows the femoral nerve 102, articular branches 104 from femoral nerve 102, an obturator nerve 132, and articular branches 134 from the obturator nerve 132. One, some, or all of the articular branches 104, 114, 124, 134 and/or one, some, or all of the nerves 102, 112, 122, 132 may be denervated to treat hip pain, several of which are described in additional detail herein. In some embodiments, the method does not include modulating a sensory nerve. In some embodiments, the method does not include modulating a motor nerve.

FIG. 2 is a schematic drawing revealing articular branches 124 of a sciatic nerve 122 supplying a dorsal portion of hip joint capsule 202 (right hip, lateral view). FIG. 2 shows the caudal part of m. gemelli 204, but the cranial part of mm. gemelli are not shown. The m. obturator internus 206 and the m. gluteus profundus 208 are shown elevated from the bone. For context, FIG. 2 also shows the body of ilium 210, the greater sciatic notch 212, the m. rectus femoris 214, and the greater trochanter 216. The terminus of the articular branch 124 is noted by the arrow A. The junction between the sciatic nerve 122 and the articular branch 124 is noted by the arrow B.

In some embodiments, a method of modulating an articular nerve branch 124 of a sciatic nerve 122 comprises locating a point on the sciatic nerve 122 as the sciatic nerve 122 passes through a caudal margin of a greater sciatic notch 212. The articular nerves 124 branch off after the point, and run to the dorsal portion of the hip joint capsule 202. The method further comprises modulating (e.g., denervating, ablating, etc.) the articular nerve branch 124.

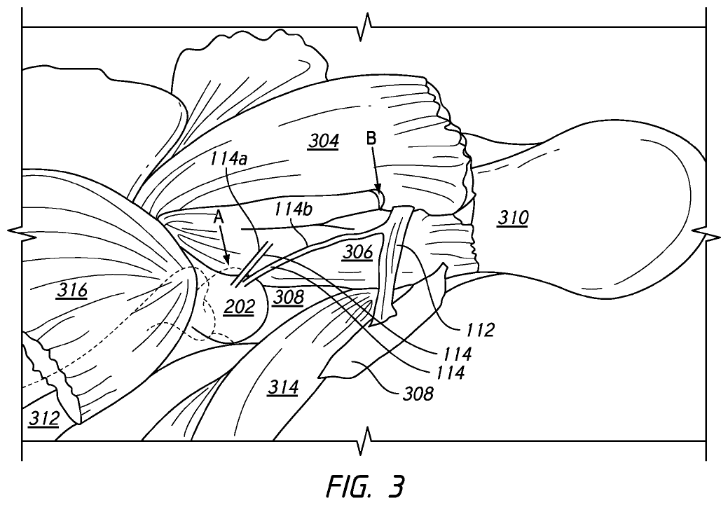

FIG. 3 is an illustration depicting innervation of a craniolateral section of a hip joint capsule 202 by articular nerve branches 114 emanating from a cranial gluteal nerve 112 (right hip, lateral view). The cranial gluteal nerve 112 penetrates between the m. gluteus medius 304 and m. gluteus profundus 306 and ends at the m. tensor fascia latae 308. The terminus of the articular branches 114 is noted by the arrow A. The junction between the gluteal nerve 112 and the articular branch 114 is noted by the arrow B. The junction noted by the arrow B may arise between the accessory head of the m. gluteus medius and the m. gluteus profundus. For context and clarity, FIG. 3 also illustrates the ilium 310, the femur 312, the m. rectus femoris 314, and the m. vastus lateralis 316, and the m. sartorius and m. articularis coxae are not shown. The cranial gluteal nerve 112 runs along the periosteum of the ilium 310 and enters the lateral muscles of the rump. The articular branch 114 may run vertically and caudally along the ventral fascia of the rump muscle before entering the hip joint capsule 202.

In several embodiments, a method of modulating an articular nerve branch 114 of a cranial gluteal nerve 112 comprises locating a junction B between the articular nerve branch 114 and a m. gluteus medius 304 and a m. gluteus profundus 306 after the articular nerve branch 114 enters lateral muscles of a rump. The articular nerve branch 114 may comprise a first articular branch 114a running between a periosteum and fascia of the m. gluteus profundus 306 running to a craniolateral aspect of the hip joint capsule 202. A second articular branch 114b can be identified before the cranial gluteal nerve 112 plunges into the m. tensor facia latae 308. The second articular branch 114 arises between the accessory head of the m. gluteus medius 304 and the m. gluteus profundus 306, and runs vertically and caudally along the ventral fascia of the rump muscle entering the craniolateral hip joint capsule and m. articularis coxae. The method further comprises modulating the articular nerve branches 114, for example as the articular nerve branches 114 enter a craniolateral aspect of the hip joint capsule 202.

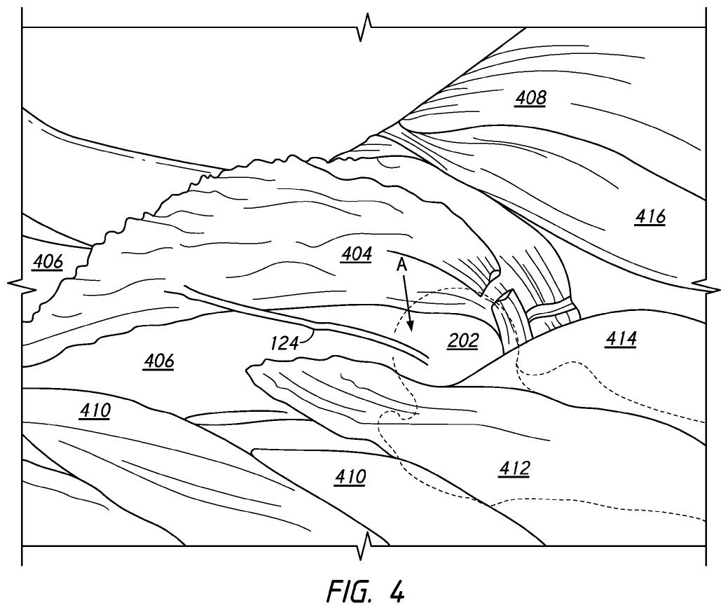

FIG. 4 is a diagram showing an articular branch 104 of a femoral nerve 102 (FIGS. 1A and 1B) innervating a cranioventral part of a hip joint capsule 202 by passing through the m. iliopsoas 404 (right hip with femur in full extension, ventral view). The terminus of the articular branch 104 is noted by the arrow A. For context and clarity, the m. iliopsoas 402 in FIG. 4 is partially severed from its original site, and FIG. 4 also shows an iliac body 406 (the m. iliopsoas 402 is elevated from the iliac body 406), mm. adductores 408, m. sartorius 410, m. rectus femoris 412, m. vastus medialis 414, and m. pectineus 416, and the femoral nerve and the femoral vessels are not shown.

In several embodiments, a method of modulating an articular nerve branch 104 of a femoral nerve 102 comprises locating a proximal portion of the femoral nerve 102 as the femoral nerve 102 passes caudoventrally through the m. iliopsoas 402. The articular branch 104 traverses distally to the caudal portion of the m. iliacus and then emerges out of the muscle, coursing a short distance between the fascia and the ventral periosteum of the ilium until reaching the cranioventral section of the hip joint capsule 402. The method further comprises modulating (e.g., denervating, ablating, etc.) the articular nerve branch 104.

FIG. 5 is a schematic drawing presenting an articular branch 134 of an obturator nerve 132 (right hip, caudoventral view with partial excision of pubis). The obturator nerve 132 passes through the obturator foramen. The terminus of the articular branch 134 is noted by the arrow A. The junction between the obturator nerve 132 and the articular branch 134 is noted by the arrow B. For context and clarity, FIG. 5 also shows a caudoventral portion of a hip joint capsule 202, muscular branches 504 of the obturator nerve 132 to mm. adductores, muscular branches 506 of the obturator nerve 132 to m. gracillus, m. obturator internus 508 separating from the pubis margin, and m. obturator externus 510 with partial resection, and branches of the medial circumflex femoral vessels are not shown.

In several embodiments, a method of modulating an articular nerve branch 134 of an obturator nerve 132 comprises following the obturator nerve 132 as the obturator nerve 132 passes through a cranial margin of obturator foramen, adjacent to a caudal portion of a hip joint capsule 202, into the obturator foramen. The articular nerve branch 134 emanates from the obturator nerve 132 when the obturator nerve 132 arrives into the foramen but before the obturator nerve 132 splits to form the muscular branches 504, 516 that feed into mm. adductores and mm. gracillus, respectively, and feeds into a caudal aspect of the hip joint capsule 202. The method further comprises modulating (e.g., denervating, ablating, etc.) the articular nerve branch 134.

In humans, the obturator nerve supplies the greatest part of the anterior capsule of the hip joint and is implicated as the cause of the groin and thigh portion of hip pain. Denervation of the obturator nerve plays an important role in managing of human hip pain. The Applicant has discovered that a connection between the obturator nerve and the source of hip pain in non-human animals, such as canine osteoarthritis, is not as strong such that denervation of the obturator nerve may not fully remedy hip pain or even play a significant role in addressing hip pain. In quadrupeds, access to the obturator nerve 132 may be difficult due to the anatomy. In some embodiments, the method does not include modulating the obturator nerve 132 or the articular branches 134 of the obturator nerve.

In several embodiments, methods are provided for treating joint pain associated with a hip of a subject. The articular joint (hip joint) is innervated by articular branch nerves originating or emanating from four nerves (cranial gluteal nerve, femoral nerve, sciatic nerve, and/or obturator nerve). In some embodiments, for example for treating quadrupeds, the method comprises identifying at least a portion of at least one of the four nerves that contribute to the circumambiency innervation of hip capsule and isolating the articular nerves at a location external to the joint capsule. In some embodiments, the method comprises modulating (e.g., denervating, ablating, etc.) the articular branch nerves at the external location with a neuromodulation device (e.g., energy delivery device) to treat pain associated with the hip.

In some embodiments, a method of modulating an articular nerve branch comprises identifying at least a portion of the articular nerve branch. Identifying the portion of the articular nerve branch comprises locating a nerve trunk and locating a junction between the articular nerve branch and the nerve trunk. The method further comprises modulating the articular nerve branch at or near the junction with the nerve trunk.

In some embodiments the method comprises identifying at least a portion of the articular nerves associated with a joint of the hip and isolating the articular nerve at a location external to the hip joint, comprising modulating (e.g., denervating, ablating, etc.) the articular nerve at the location external to the hip joint to treat the pain associated with the hip.

Several embodiments include a method for treating joint pain associated with a hip of a subject. The hip is innervated by articular branches originating from the cranial gluteal nerve, the sciatic nerve, the femoral nerve, and/or the obturator nerve. In some embodiments, the method comprises percutaneously guiding a delivery device within or near the hip joint. In some embodiments, the method comprises identifying at least a portion of the cranial gluteal nerve, femoral nerve, sciatic nerve, or obturator nerve associated with the hip joint and isolating the articular branch nerves at a location external to the hip joint. In some embodiments, the method comprises delivering a treatment device to the external location using the delivery device and operating the treatment device at the external location. In some embodiments, the operation of the treatment device is configured to modulate the cranial gluteal nerve, the femoral nerve, the sciatic nerve, or the obturator nerve at the external location to treat pain associated with the hip joint. In some embodiments, the delivery device comprises a catheter comprising a first lumen for advancing an imaging device and a second lumen for advancing the treatment device.

In some embodiments, determining a target treatment site comprises acquiring a hip joint reference point corresponding to a radiographically identifiable anatomical feature of the hip joint and generating coordinates for the target treatment site as a function of a calculated distance from the reference point. For example, the calculated distance may correspond to a predicted articular nerve location that is obtained (e.g., automatically obtained) from analysis of the acquired imaging data.

Treatment may be delivered to the target treatment site to modulate at least a portion of an articular nerve. The target treatment site may be, for example, a terminus of the articular nerve, a junction between the articular nerve and the nerve, and/or a portion of the articular nerve between the terminus and the junction. In accordance with several embodiments, the treatment may focus on a location of the articular nerve that is upstream (closer to the nerve junction, further from the terminus) of the articular nerve. In some embodiments, a method may comprise modulating (e.g., denervating, ablating, etc.) one or more nerves instead of or in addition to articular nerves. In some embodiments, a method does not include modulating a nerve.

In human beings, the sensitive innervation of the acetabular area includes branches from the superior gluteal, sciatic or ischiatic, femoral, and obturator nerves. In dogs, as an example of a quadruped, the sensitive innervation of the acetabular area includes the cranial gluteal, sciatic, and femoral nerves. The obturator nerve is not particularly related to this function in most non-human animals such that it can be considered an anatomic variation. The innervation, both in human beings and non-human animals, presents bilateral symmetry and, apart from that, the difference between the human bipedal support and the canine quadrupedal support generates biomechanical forces in distinct points, leading to different nerve fiber concentrations between the two species. While humans may present a bigger nerve fiber density in the anteromedial area, for which the obturator nerve is responsible, dogs present a bigger density in the craniolateral and dorsal areas, for which the cranial gluteal and the sciatic nerves are most responsible for pain, followed by the femoral nerve and the obturator nerve.

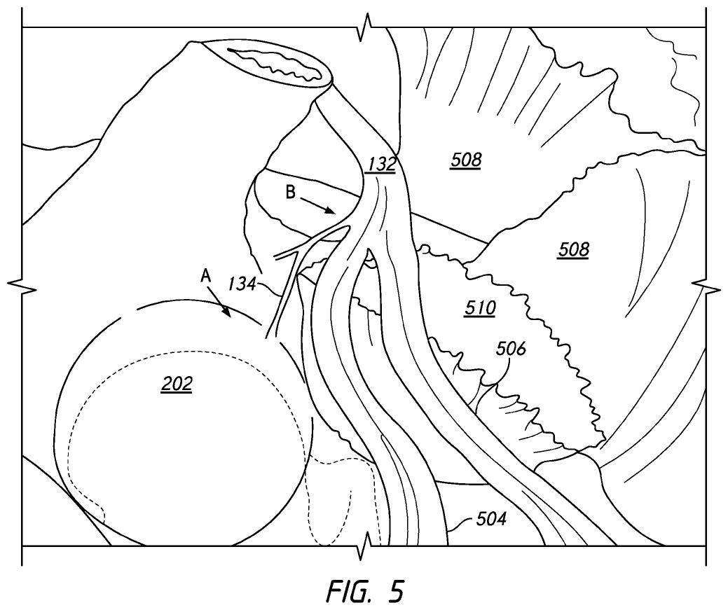

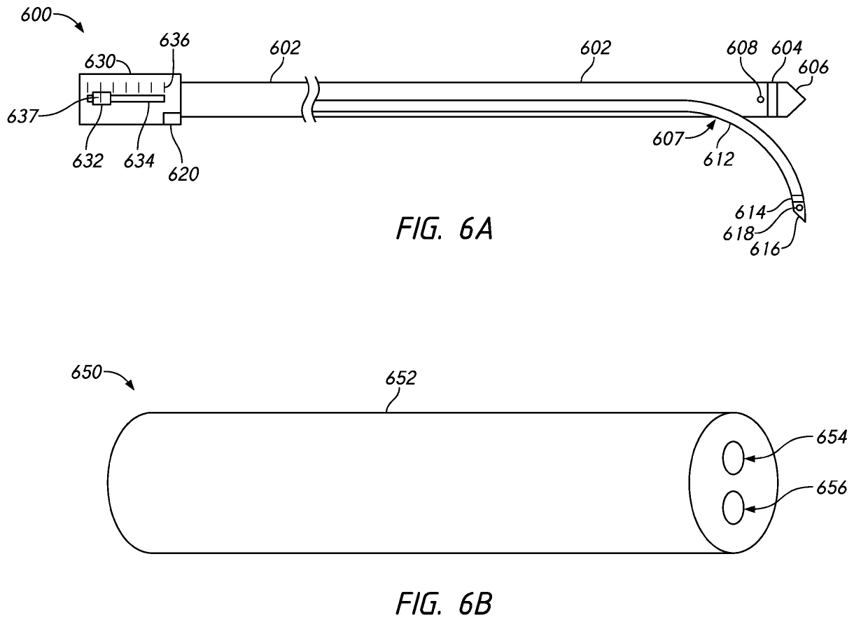

FIG. 6A schematically illustrates an example treatment device 600. The treatment device 600 comprises a needle body 602. The needle body 602 may comprise and one or more suitable biocompatible materials, for example, nitinol, chromium cobalt, Elgiloy, MP35N, Finox, Phynox, stainless steel, metal alloys, fiberglass, carbon fiber, etc. The first electrode 604 may comprise radiopaque material. The needle body 602 may comprise a radiopaque marker. For example, the tip 606 may comprise radiopaque material for visualization under fluoroscopy. The needle body 602 may comprise an echogenic surface. For example, the tip 606 may comprise an echogenic surface for visualization under ultrasound. In some embodiments, the tip 606 comprises radiopaque material and an echogenic surface. In contrast to direct visualization, for example as in open surgery, articular branches may be visualized which can provide targeted modulation of the desired nerves and not undesirably damage or remove a great deal of adjacent tissue.

The treatment device 600 comprises a first electrode 604 along the needle body 602. The first electrode 604 may at least partially span a circumference or a surface of the needle body 602. In various embodiments, the needle body 602 may have a round, elliptical, oval, smooth, edged, faceted, symmetrical, asymmetrical cross-section, with one, two, three, four, five, six, or more sides. In some embodiments, the first electrode 604 comprises a barrel electrode. In some embodiments, the first electrode 604 comprises a button electrode. In some embodiments, the first electrode 604 comprises an uninsulated or a partially insulated metal portion of the needle body 602. The first electrode 604 is proximate to a distal end of the needle body 602. For example, the first electrode 604 may be between about 0.1 mm and about 2 mm (e.g., about 0.1 mm, about 0.2 mm, about 0.3 mm, about 0.4 mm, about 0.5 mm, about 1 mm, about 1.5 mm, about 2.5 mm, and ranges between such values) from the distal end of the needle body 602. The distal end of the needle body 602 comprises a tissue-penetrating tip 606. The tip 606 is configured to allow the treatment device 600 to be inserted to a target site percutaneously or minimally invasively. In some embodiments, the procedures described herein, using the treatment device 600 or other treatment devices, can avoid or reduce open surgery and its myriad complications such as high infection risk, long recovery time of a 3-5 cm incision including all of the tissue between the skin and the bone, damaging or removing the entire periosteum, etc. The tip 606 may comprise, for example, a beveled tip (e.g., single bevel or multi-bevel), a pencil-point tip (e.g., multi-faceted or conical), etc. In one embodiment, the treatment device 600 comprises a first temperature sensor 608 configured to sense a temperature proximate to the first electrode 604. The first temperature sensor 608 is shown proximal to the first electrode 604. Positioning the first temperature sensor 608 proximal to the first electrode 604 can reduce the distance between the first electrode 604 and the distal end of the tip 606 because there are fewer structures between the first electrode 604 and the distal end of the tip 606. Positioning the first temperature sensor 608 proximal to the first electrode 604 can be useful when power control is used for modulation because the temperature can be monitored closer to structures that are not trying to be treated (e.g., outside of an ablation zone). The first temperature sensor 608 may be distal to the first electrode 604. If the first electrode 604 is not fully annular, the first temperature sensor 608 may be longitudinally aligned with the first electrode 604.

The needle body 602 comprises one or more apertures 607. In one embodiment, the one or more apertures 207 are located on a lateral surface of the needle body 602. The needle body 602 may comprise a longitudinally-extending lumen in communication with the aperture 607. The needle body 602 may be hollow at least proximal to the aperture 607. The treatment device 600 comprises a stylet 612 configured to exit the aperture 607 and curve radially away from the needle body 602. The treatment device 600 comprises a stylet 612 configured to exit the lateral aperture 607 and extend laterally away from the needle body 602 at an angle non-parallel to a longitudinal axis of the needle body 602. In various embodiments, the stylet 612 extends laterally away from the needle body at an angle of 20-160 degrees (e.g., 30°-140°, 45°-135°, 60°-120°, 80°-110°, 20°, 25°, 30°, 35°, 40°, 45°, 50°, 55°, 60°, 65°, 70°, 75°, 80°, 85°, 90°, 95°, 100°, 110°, 115°, 120°, 125°, 130°, 135°, 140°, 145°, 150°, 155°, 160°, and any range or value of angles therein). In one embodiment, the stylet 612 extends with a curve such that the distal tip of the stylet extends perpendicularly from the needle body 602. The aperture 607 may be free of or lack a ramped surface. In one embodiment, the aperture 607 comprises a ramped surface over which the stylet is configured to slide in order to extend at an angle laterally or radially away from the needle body 602. In one embodiment, the ramped surface proximate the aperture 607 is adjustable (e.g., with actuatable with a knob or lever or button) to controllably change the angle of lateral deployment extending the stylet 612 from the needle body 602. The stylet 612 may comprise one or more suitable biocompatible materials, for example, nitinol, chromium cobalt, Elgiloy, MP35N, Finox, Phynox, stainless steel, metal alloys, etc. The stylet 612 comprises a shape memory material so that the stylet 612 can be heat treated or shape set to have a curvature such that the stylet 612 radially extends from the aperture 607 along a curved path. The stylet 612 may comprise a shape memory material that, when the treatment device 600 is sterilized, reverts to a set curved shape, which may be able to reduce or eliminate bends in the stylet 612. The aperture 607 may be used to infuse liquid (e.g., saline, contrast, etc.) into a modulation site. For example, the modulation may comprise RF ablation enhanced with liquid.

The treatment device 600 comprises a second electrode 614 along the stylet 612. In various embodiments, the stylet 612 may have a round, elliptical, oval, smooth, edged, faceted, symmetrical, asymmetrical cross-section, with one, two, three, four, five, six, or more sides. The second electrode 614 may at least partially span a circumference or surface of the stylet 612. In some embodiments, the second electrode 614 comprises a barrel electrode. In some embodiments, the second electrode 614 comprises a button electrode. In some embodiments, the second electrode 614 comprises an uninsulated metal portion of the stylet 612. The second electrode 614 is proximate to a distal end of the stylet 612. The distal end of the stylet 612 comprises a tissue-penetrating tip 616. The tip 616 may comprise, for example, a beveled tip (e.g., single bevel or multi-bevel), a pencil-point tip (e.g., multi-faceted or conical), etc. In some embodiments, a beveled tip 616 that is angled to face the proximal end of the device 600 upon curving can deflect the stylet 612 distally as the stylet 612 is distally advanced. In various embodiments, the tip 606 is configured to contact a bone (e.g., the ilium bone) and then the stylet 612 is configured to be distally advanced, a beveled tip 616 can help the distal part of the stylet 612 scrape along the surface of the bone. If the bevel faced distally, then the stylet 612 might be inclined to lift away from the bone. In some embodiments, a method of manufacturing the stylet 612 comprises forming a beveled tip 616 and shape setting a curve into the stylet 612 so that the beveled tip faces proximally upon curving. The curvature can help the stylet to extend substantially radially away from the needle body 602, for example with a small longitudinal component so that the first electrode 604 and the second electrode 614 can both be at a substantially similar longitudinal extent (e.g., along a bone).

The second electrode 614 may comprise radiopaque material. The stylet 612 may comprise a radiopaque marker. For example, the tip 606 may comprise radiopaque material for visualization under fluoroscopy. The stylet 612 may comprise an echogenic surface. For example, the tip 606 may comprise an echogenic surface for visualization under ultrasound. In some embodiments, the tip 606 comprises radiopaque material and an echogenic surface. Other radiopaque and/or echogenic markers are also possible. For example, the needle body 602 may comprise a radiopaque, echogenic, and/or visual marker to help a user identify the aperture 607 (e.g., proximate the aperture 607, around the aperture 607, on an opposite side of the aperture 607, etc.) to determine a direction of extension of the stylet 612. Fluoroscopy may be used to view radiopaque markers of the device 600 during and/or after insertion into the subject, for example to confirm position prior to modulation. Ultrasound may be used to view echogenic surfaces of the device 600 during and/or after insertion into the subject, for example to confirm position prior to modulation. The ultrasound may be external ultrasound. In some embodiments, the needle body 602 has a circular cross-section. In some embodiments, the needle body 602 has a non-circular cross-section, which can provide a user with rotational orientation information in the absence of a marker per se. For example, the needle body 602 can have a D-shaped cross-section in which the aperture 607 is along the flat side, opposite the flat side, etc.

The treatment device 600 comprises a second temperature sensor 618 configured to sense a temperature proximate to the second electrode 614. The second temperature sensor 618 is shown distal to the second electrode 614. Positioning the second temperature sensor 618 distal to the second electrode 614 does not affect the distance between the second electrode 614 and the distal lateral surface that the stylet 612 extends along. Positioning the second temperature sensor 618 distal to the second electrode 614 can be useful when power control is used for modulation because the temperature can be monitored closer to structures that are not trying to be treated (e.g., outside of an ablation zone). The second temperature sensor 618 may be proximal to the second electrode 614. If the second electrode 614 is not fully annular, the second temperature sensor 618 may be longitudinally aligned with the second electrode 614. The treatment device 600 may comprise the first temperature sensor 608, the second temperature sensor 618, or both the first temperature sensor 608 and the second temperature sensor 618. One temperature sensor may help to reduce costs and/or complexity of the treatment device 600. Two temperature sensors may help to provide information about specific local temperature (e.g., due to different heating proximate to the first electrode 604 versus the second electrode 614 due to nearby anatomy). Two temperature sensors may help to show an error in one of the temperature sensors. Other temperature sensors are also possible (e.g., proximate the aperture 607). The temperature sensor(s) may be used to provide feedback to a system that can be used, for example, to determine a treatment duration.

During modulation, the first electrode 604 may be an anode and the second electrode 614 may be a cathode, or vice versa, to provide bipolar modulation. The energy extends between the first electrode 604 and the second electrode 614 to create a line of ablation. In some embodiments, the treatment device 600 is configured to create a substantially cylindrical or thin prolate spheroid ablation zone. In some embodiments, the treatment device 600 does not create a spherical, egg-shaped, oval, etc. ablation zone. In one embodiment, the modulation is not monopolar and does not use a grounding pad.

Other treatment devices are also possible. For example, the treatment device may comprise a needle and a plurality of stylets each comprising an electrode. Depending on positioning, one electrode on one stylet may be made anodic and one electrode on another stylet may be made cathodic so that the ablation zone is between the electrodes on two stylets (e.g., as opposite to being between an electrode on a needle body and an electrode on a stylet). If the stylets are configured to travel along the surface of a bone, for example, the ablation zone may be able to substantially avoid tissue distant from the bone. In some embodiments, a plurality of electrodes on different stylets may be used as the anode and/or the cathode, which can make generally flat polygonal shaped ablation zones, which could be useful for denervating a larger area where nerves extend in different directions. For another example, the treatment device may comprise a monopolar electrode. The monopolar electrode, for example when placed accurately relative to a nerve, can accurately ablate the nerve. A monopolar electrode may comprise a cooling system, for example if a larger ablation zone is desired. In some embodiments, the treatment device may be implantable. An implantable treatment device may advantageously modulate the nerves to inhibit or prevent transmission of pain signals, but keep the nerves intact. An implantable device may comprise a power source (e.g., wirelessly rechargeable battery) and a lead or a plurality of leads including electrodes configured to be positioned proximate the articular branch nerves of interest. In some embodiments, the modulation does not denervate or ablate tissue.

Although described herein with respect to RF (including bipolar, monopolar, liquid enhanced, etc.), other energy modalities may also or alternatively be used via, for example, a treatment device, such as unfocused ultrasound, focused ultrasound such as high-intensity or low-intensity focused ultrasound, microwave energy, thermal energy (e.g., cryoenergy, heat or cold provided by a fluid (e.g., water, saline, liquid medicament, etc.) or gas (e.g., steam)), electrical energy (e.g., non-RF electrical energy), infrared energy, laser energy, phototherapy or photodynamic therapy (e.g., in combination with one or more activation agents), plasma energy (e.g., plasma blades), ionizing energy delivery (e.g., X-ray, proton beam, gamma rays, electron beams, alpha rays, etc.), electroporation (e.g., irreversible electroporation), mechanical energies delivered by cutting or abrasive elements, cryoablation, chemical energy or modulation (e.g., chemoablation), or combinations thereof. In some embodiments, disruption or interruption of nerves is carried out by chemicals or therapeutic agents (for example, via drug delivery), either alone or in combination with an energy modality. In some embodiments, pharmaceuticals are combined with the neuromodulation (e.g., ablation) described herein to reduce the dosage or duration of pharmacology therapy, thus reducing side effects. In various embodiments, different energy modalities may be used in combination (either simultaneously or sequentially).

In some embodiments, the device 600 comprises a handle 630. The handle 630 is configured to couple to a nerve modulation system (e.g., by connecting the treatment device 600 to a connector). The handle 630 may comprise, for example, electrical couplers and/or fluid couplers. The handle 630 comprises a mechanism for deploying the stylet 612 from the needle body 602. FIG. 6A shows an example deployment mechanism comprising a knob 632 configured to slide in a channel 634. Distally advancing the knob 632 distally advances the stylet 612. The movement may be direct, or gearing may be used such that the movement of the knob 632 is larger than the movement of the stylet 612 or smaller than the movement of the stylet 612. The handle 630 may comprise indicia 636, for example alignable with indicia 637 on the knob 632, to provide a user with information about the extent of extension of the stylet 612. In some embodiments, the handle 630 comprises sensors configured to communicate extension information with a nerve modulation system.

In some embodiments, the handle 630 comprises a rotatable knob. Gearing (e.g., a worm gear, bevel gears, rack and pinion, etc.) can be used to translate the rotation of the knob into longitudinal movement of the stylet 612. The handle may comprise detents (e.g., providing audible and/or tactile feedback), indicia, sensors, etc. to provide a user with information about the extent of extension of the stylet 612. In some embodiments, the knob 632 may be longitudinally advanced to provide rough or gross motion and the knob 632 may be rotated to provide fine motion.

In some embodiments, the handle 630 is rotatable relative to the needle body 602. Gearing (e.g., a worm gear, bevel gears, rack and pinion, etc.) can be used to translate the rotation of the handle 630 into longitudinal movement of the stylet 612. The handle may comprise detents, indicia, sensors, etc. to provide a user with information about the extent of extension of the stylet 612.

The stylet 612 may be replaceable, for example by being removably coupled to the handle 630. If the stylet 612 is bent beyond a useful tolerance, the existing stylet 612 can be removed and a second stylet 612 can be inserted into the needle body 602 and coupled to the handle 630. The stylet 612, the handle 630, and/or the needle body 602 may comprise keyed or alignment features to ensure that the second stylet 612 exits the aperture 607 along a curved path upon longitudinal movement.

FIG. 6B schematically illustrates an example delivery device 640 for a treatment device 600. The delivery device 640 comprises a catheter 642 comprising a first lumen 644 and a second lumen 646. In one embodiment, delivery device 650 comprises a catheter 652 comprising a first lumen 654 and a second lumen 656. In some embodiments, one, two, three, four or more lumens may be included. An imaging device (e.g., an optical scope, an ultrasound scanner, or combinations thereof, etc.) can be advanced through the first lumen 644, 654 to a treatment site. The lumen 644, 654 may be used to supply fluid (e.g., ultrasound fluid, saline, contrast, etc.) to the treatment site. The imaging device may be used to image the treatment site. A treatment device (e.g., the treatment device 600 and/or other treatment devices) can be advanced through the second lumen 646, 656 to the treatment site. The imaging device may be used to image the treatment device. A system may comprise the treatment device 600 and the delivery device 640, 650. The delivery device 640, 650 may be configured to rest on the surface of a subject (e.g., to not penetrate the skin of a subject). In certain such embodiments, the lumens 644, 654, 646, 656—may help to position the imaging device and the treatment device in a particular orientation, spacing, etc.

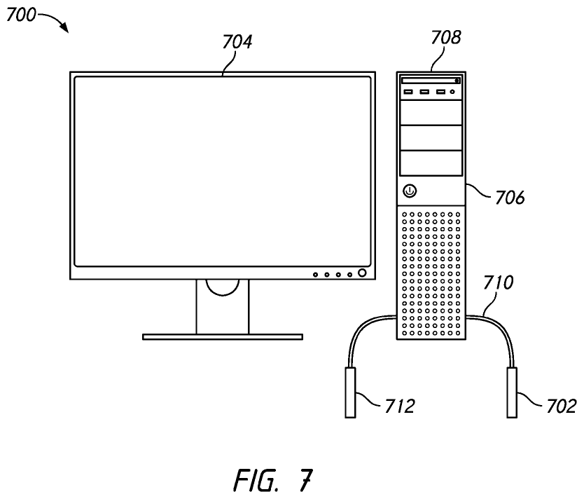

FIG. 7 is a schematic diagram of an example tissue treatment (e.g., nerve modulation) system 700. The system 700 serves as the user interface and provides the energy to a treatment device 702. The system 700 includes a display screen 704, energy generator 706, a control computer 708, and a removable connector 710 between the control computer 706 and the treatment device 702. The system 700 optionally comprises an imaging device 712. The display screen 702 may be a touch screen. The system 700 may comprise other inputs (e.g., a mouse, a keyboard, a track ball, foot pedal, etc.). In some embodiments, the control computer 708 may comprise the energy generator 706 or vice versa, or the energy generator 706 and the control computer 708 may be integral. Referring again to FIG. 6B, the imaging device 712 may be configured to be advanced through the first lumen 654 and the treatment device 702 is configured to be advanced through the second lumen 606. A system may comprise the system 700, the treatment device 600, and the delivery device 650. A system may comprise the system 700 and the treatment device 600. A system may comprise the system 700 and the delivery device 650. The treatment device 702 may comprise one or more electrodes configured to generate radiofrequency (RF) energy to ablate tissue around the one or more electrodes. The RF may be monopolar or bipolar. The imaging device 712 may comprise a laparoscope, an ultrasound imager, etc. The display screen 702 may show images from the imaging device 712. The images may be manually and/or automatically annotated, for example to mark anatomical landmarks, show an expected ablation area, etc.

The treatment device may be configured to apply bipolar RF energy to an anode and a cathode at a frequency between about 100 kilohertz (kHz) and about 100 megahertz (MHz) (e.g., about 100 kHz, about 250 kHz, about 450 kHz, about 500 kHz, about 550 kHz, about 1 MHz, about 10 MHz, about 50 MHz, about 100 MHz, and ranges between such values), a power between about 0.1 Watts (W) and about 100 W (e.g., about 0.1 W, about 1 W, about 5 W, about 10 W, about 15 W, about 20 W, about 25 W, about 50 W, about 75 W, about 100 W, and ranges between such values), a current between about 0.5 milliamperes (mA) and about 5 amperes (A) (e.g., about 0.5 mA, about 1 mA, about 10 mA, about 100 mA, about 500 mA, about 1 A, about 1.5 A, about 2 A, about 3 A, about 4 A, about 5 A, and ranges between such values) (if measured as root mean squared (rms), a current between about 0.25 mAᵣₘₛ and about 3 Aᵣₘₛ (e.g., about 0.25 mAᵣₘₛ, about 0.5 mAᵣₘₛ, about 1 mAᵣₘₛ, about 10 mAᵣₘₛ, about 100 mAᵣₘₛ, about 500 mAᵣₘₛ, about 750 mAᵣₘₛ, about 1 Aᵣₘₛ, about 1.5 Aᵣₘₛ, about 2 Aᵣₘₛ, about 2.5 Aᵣₘₛ, about 3 Aᵣₘₛ, and ranges between such values), a duration between about 1 second(s) and about 20 minutes (min) (e.g., about 1 s, about 3 s, about 5 s, about 10 s, about 15 s, about 20 s, about 30 s, about 45 s, about 1 min, about 2 min, about 3 min, about 4 min, about 5 min, about 10 min, about 15 min, about 20 min, and ranges between such values), etc. For example, a non-limiting example treatment energy may have a frequency between about 450 kHz and about 500 kHz, a power between about 10 W and about 20 W, a current between about 1 A and about 2 A, and a duration between about 30 s and about 5 min. Stimulation may be pulsed for a portion of the duration (e.g., a few seconds at a time) and turned off or not applied for other portions of the duration. Other parameters, for example depending on the specific treatment device 702, are also possible. In some embodiments, the energy has a waveform. In various embodiments, the energy waveform is varied.

The treatment device 600 can be used to perform one ablation or a plurality of ablations. The treatment device 600 may be sterilizable for use on a plurality of subjects. The treatment device 600 may comprise electronic circuitry 631 schematically shown in FIG. 6A as being in the handle 630, but the electronic circuity 631 can be located at any suitable position on the treatment device 600. The electronic circuitry 631 may be configured to track use information (e.g., the number of uses of the treatment device 600, the number of times the treatment device 600 is sterilized (indicative of the number of procedures) (e.g., using thermocouple information to sense high temperature, time between treatments, etc.), performance data (e.g., duration of use), defects, power used, etc.), which can be used for billing users by the number of uses. For example, whenever the treatment device 600 is coupled to a system 700 (e.g., by connecting the treatment device 600 to the connector 710), the use information may be transmitted locally and/or over a network where a supplier can access the information for billing purposes. In some embodiments, the connector 710 may comprise the electronic circuitry 631. In certain such embodiments, upon connection of the treatment device 702 to the connector 710, a usage may be measured. The electronic circuitry 631 may derive power from the system 700. The electronic circuitry 631 may comprise, for example, a processor and/or memory. In embodiments in which the treatment device 600 comprises the electronic circuitry 631, the electronic circuitry 631 may comprise a power source (e.g., for detecting information when not coupled to the system 700). The components of the electronic circuitry 631 may be configured to withstand sterilization. The electronic circuitry 631 may comprise insulation to at least partially shield the electrical components of the electronic circuitry 631 from sterilization.

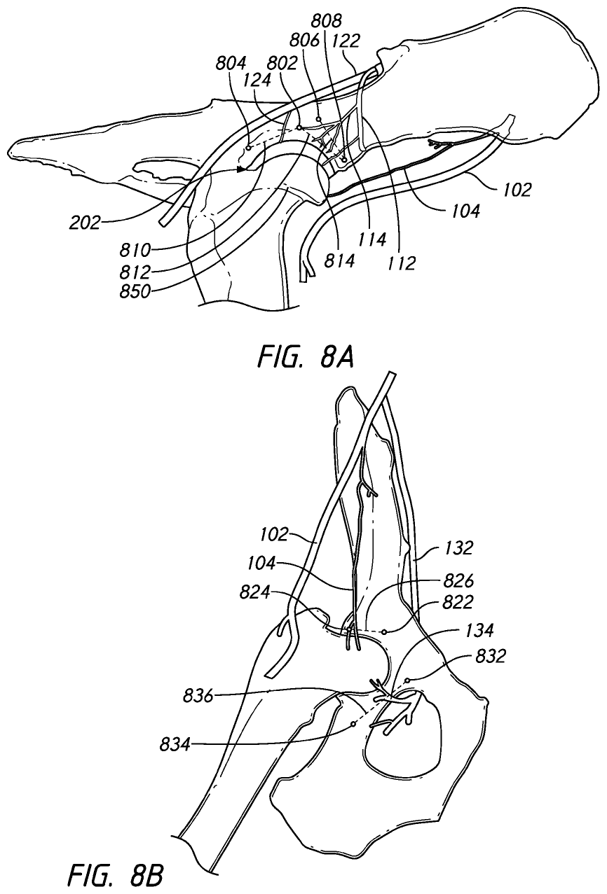

FIGS. 8A and 8B illustrate an example method of treating hip joint pain. The method comprises modulating (e.g., denervating, ablating, etc.) an articular branch 124 from the sciatic nerve 122 and an articular branch 114 from the cranial gluteal nerve 112. The method optionally comprises modulating an articular branch 104 from femoral nerve 102. The method optionally comprises modulating an articular branch 134 from the obturator nerve 132.

Using a dorsal and caudal approach, a user can insert a treatment device at a first point 802. The first point 802 may be an apex of a hip joint capsule. The user can advance the treatment device until the distal end contacts bone. Other approaches are also possible. Facets of the hip joint capsule 202 may be used to help determine a position of the first point 802. If the treatment device comprises the treatment device 600, for example, imaging can help to orient the treatment device 600 so that the stylet 612 is deployed until the second point 804. The acetabulum/acetabular rim (socket) and femoral head 850, for example, may be used to help determine a position of the second point 804. Imaging (e.g., fluoroscopy, ultrasound, etc.) may be used to verify the position of the treatment device prior to modulation. Terms such as “first,” second, “third,” etc. provide nomenclature that can help distinguish between different points or other multiple items discussed herein, and do not necessarily describe an order, a preference, a hierarchy, etc. For example, in some embodiments, the treatment device may be inserted at the second point 804 and extended to the first point 802. For example, the second point 804 may be easier to identify than the first point 802 when percutaneously positioning the treatment device. The acetabulum is bounded dorsally, cranially, and caudally by the acetabular rim. The targeted modulations may occur just above this rim when targeting the articular branch 124 of the sciatic nerve 122 and/or the articular branch 114 of the cranial gluteal nerve 112. Using borders (e.g., dorsal, cranial, caudal) of the hip joint or acetabulum can help to identify the rim, which may be termed the dorsal rim and/or the ventral rim). Although certain examples of insertion and extension points are provided herein, other insertion and extension points are also possible, for example others that would create a path that would include an articular branch nerve of interest. Bipolar radiofrequency energy can be applied between the first electrode 604 and the second electrode 614. The energy will extend along the path 810, forming a substantially linear or cylindrical ablation zone. The articular branch 124 from the sciatic nerve 122 lies in the path 810 such that the RF energy can modulate the articular branch 124. The stylet 612 can be retracted back into the needle body 602. After modulating the articular branch nerve 124, the pain is reduced. Pain reduction can be measured, for example, by walking evaluation, a biped station, rotation with external abduction, subluxation and iliopsoas, combinations thereof, a distraction index, etc. The test(s) can be performed before a procedure to establish a preoperative baseline, and then at one or more intervals after the procedure (e.g., one day, two days, one week, two weeks, one month, three months, six months, etc.). Imaging can also or alternatively be used to evaluate the tissue for signs of recovery.

In some embodiments, the user can rotate the treatment device 600 (e.g., between about 100° and about 160°) to orient the treatment device 600 so that the stylet 612 is deployed until the third point 808. Facets of the hip joint capsule 202 may be used to help determine a position of the third point 808. Imaging (e.g., fluoroscopy, ultrasound, etc.) may be used to verify the position of the treatment device prior to modulation. Bipolar radiofrequency energy can be applied between the first electrode 604 and the second electrode 614. The energy will extend along the path 812, forming a substantially linear or cylindrical ablation zone. The articular branch 114 from the cranial gluteal nerve 112 lies in the path 812 such that the RF energy can modulate the articular branch 114. After modulating the articular branch nerve 114, the pain is reduced. The stylet 612 can be retracted back into the needle body 602. Rotating the treatment device 600 between energy application can reduce the number of puncture sites, which can reduce soft tissue trauma and a number of possible infection sites. A smaller amount of rotation is also possible, for example to make sure that the first tissue was treated enough to capture the articular branch nerve.

In some embodiments, using a dorsal and caudal approach, the user can insert the treatment device at a fourth point 806. Other approaches are also possible. The user can advance the treatment device until the distal end contacts bone. If the treatment device comprises the treatment device 600, for example, imaging can help to orient the treatment device 600 so that the stylet 612 is deployed until the third point 808. Imaging (e.g., fluoroscopy, ultrasound, etc.) may be used to verify the position of the treatment device prior to modulation. In some embodiments, the treatment device may be inserted at the third point 808 and extended to the fourth point 808. Bipolar radiofrequency energy can be applied between the first electrode 604 and the second electrode 614. The energy will extend along the path 814, forming a substantially linear or cylindrical ablation zone. The articular branch 114 from the cranial gluteal nerve 112 lies in the path 812 such that the RF energy can modulate the articular branch 114. After modulating the articular branch nerve 114, the pain is reduced. The stylet 612 can be retracted back into the needle body 602. Removing and then reinserting the treatment device can provide greater flexibility in treatment, for example if extension from the first point 802 to the second point 804 is good for modulating the articular branch 124 but the anatomy makes extension from the first point 802 to the third point 808 difficult for modulating the articular branch 114. In some embodiments, a second puncture site can be useful for targeting a plurality of articular branches 114 (e.g., as show in FIG. 8A). In some embodiments, a second puncture site can be useful when the stylet 612 is extended from an edge towards a center. In some embodiments, both rotation and an additional puncture site may be used.

The lengths of the paths 810, 812, 814 are long enough to capture an articular branch to be modulated but short enough that the modulation energy can be somewhat targeted. One, some, or all of the paths 810, 812, 814 may be along a portion of the diameter of the joint, such as a hip joint, for example between about 1/10 and about ⅔ (e.g., about 1/10, about ⅛, about ⅙, about ¼, about ⅓, about ½, about ⅔, and ranges between such values). For example, in an average adult German Shepherd, the acetabulum has a diameter of about 20 mm, so one, some, or all of the paths 810, 812, 814 may have a length between about 2 mm and about 13.3 mm (e.g., about 2 mm, about 2.5 mm, about 3.3 mm, about 5 mm, about 6.7 mm, about 10 mm, about 13.3 mm, and ranges between such values). In various embodiments, length is between 0.1 mm and 60 mm, with ranges and values of 1-22 mm, 5 -25 mm, 10-30 mm, 15-30 mm, 20-30 mm, 27-30 mm, 40-50 mm, 43-48 mm, 56-58 mm, 5-50 mm, 10-40 mm, 15-30 mm, 30-60 mm, 30-50 mm, 30-45 mm, 30-55 mm, 40-60 mm, 50-60 mm, 35-55 mm, 35-60 mm, 40-55 mm, 45-60 mm, 55-60 mm, or other ranges and values therein. For another example, in an average adult horse, the acetabulum has a diameter of about 56 mm, so one, some, or all of the paths 810, 812, 814 may have a length between about 5 mm and about 37 mm (e.g., about 5 mm, about 6 mm, about 7 mm, about 9 mm, about 14 mm, about 19 mm, about 28 mm, about 37 mm, and ranges between such values). For another example, in an average adult human male, the acetabulum has a diameter of about 52 mm, so one, some, or all of the paths 810, 812, 814 may have a length between about 5 mm and about 35 mm (e.g., about 5 mm, about 6 mm, about 7 mm, about 9 mm, about 13 mm, about 17 mm, about 26 mm, about 35 mm, and ranges between such values). For another example, in an average adult human female, the acetabulum has a diameter of about 46 mm, so one, some, or all of the paths 810, 812, 814 may have a length between about 4 mm and about 31 mm (e.g., about 4 mm, about 5 mm, about 6 mm, about 8 mm, about 12 mm, about 15 mm, about 23 mm, about 31 mm, and ranges between such values). Larger breeds or species (e.g., bovine, equine, large feline, pachyderm) may have longer paths 810, 812, 814. Smaller breeds or species (e.g., feline) may have shorter paths 810, 812, 814. The stylet 612 may be configured to treat both large and small breeds of one type of species. The stylet 612 may be configured to treat both large and small breeds and species.

In various embodiments, the length of a treatment, e.g., such as a distance between electrodes, is a value or an adjustable value in a range of about 1 mm and 100 mm (e.g., 1-50 mm, 2-100 mm, 5-50 mm, 10-60 mm, 20-80 mm, 30-90 mm, 40-70 mm, 30-50 mm, 25-35 mm, 25-30 mm, 30-35 mm, 25-50 mm, 20-50 mm, 20-60 mm, 20-70 mm, 20-75 mm, 20-80 mm, 20-90 mm, 40-60 mm, 40-70 mm, 45-60 mm, 50-100 mm, 50-75 mm, 55-75 mm, 60-80 mm, 70-95 mm, 20-100 mm, 30-100 mm, 40-100 mm, 50-100 mm, 60-100 mm, 70-100 mm, 80-100 mm, 90-100 mm, and any values and ranges therein; e.g., 1 mm, 2 mm, 2.5 mm, 3.3 mm, 4 mm, 5 mm, 6.7 mm, 7 mm, 9 mm, 10 mm, 12 mm, 13 mm, 14 mm, 15 mm, 17 mm, 19 mm, 20 mm, 23 mm, 25 mm, 26 mm, 30 mm, 31 mm, 35 mm, 37 mm, 40 mm, 43 mm, 45 mm, 48 mm, 50 mm, 56 mm, 58 mm, 60 mm, 62 mm, 67 mm, 70 mm, 75 mm, 80 mm, 90 mm, 95 mm, 100 mm and ranges and values between such values). In various embodiments, length is between 0.1 mm and 60 mm, with ranges and values of 1-22 mm, 27-30 mm, 43-48 mm, 56-58 mm, 5-50 mm, 10-40 mm, 15-30 mm, or other ranges and values therein. Larger breeds or species (e.g., canine, bovine, equine, large feline, pachyderm, such as Mastiff, Pit Bulls, Greyhounds, Shepherd, etc.) may have longer paths 810, 812, 814. Smaller breeds (e.g., Chihuahua, Shih Tzu, Maltese,) or species (e.g., feline) may have shorter paths 810, 812, 814. The stylet 612 may be configured to treat both large and small breeds of a type of species.

In various embodiments, a torque of a device such as the outer tube will be in a range of 1-6 in-lb. (e.g., 1, 1.5, 1.7, 1.9, 2, 2.1, 2.2, 2.3, 2.5, 2.8, 3, 3.3, 3.5, 3.7, 3.9, 4, 4.2, 4.5, 4.8, 5, 5.3, 5.5, 5.8 and 6 in-lb, and other ranges and values therein).

When the modulation is close to the surface of the bone and/or follows a substantially linear path, tissue such as muscles, tendons, blood vessels, etc. can be substantially avoided. For example, RF ablation around a muscle can cause muscle atrophy, but RF ablation distant to the muscles can avoid causing muscle atrophy.

Referring to FIG. 8B, additional articular branches 104 and/or 134 can be optionally modulated. Using a dorsal and caudal, a true lateral, a cranial and ventral, etc. approach a user can insert a treatment device at a fifth point 822. Other approaches are also possible. The user can advance the treatment device until the distal end contacts bone. Facets of the hip joint capsule, the acetabular notch, blood vessels, and/or other anatomical landmarks may be used to help determine a position of the fifth point 822. If the treatment device comprises the treatment device 600, for example, imaging can help to orient the treatment device 600 so that the stylet 612 is deployed until the sixth point 824. Facets of the hip joint capsule, blood vessels, and/or other anatomical landmarks may be used to help determine a position of the sixth point 824. Imaging (e.g., fluoroscopy, ultrasound, etc.) may be used to verify the position of the treatment device prior to modulation. Bipolar radiofrequency energy can be applied between the first electrode 604 and the second electrode 614. The energy will extend along the path 826, forming a substantially linear or cylindrical ablation zone. The articular branch 104 from the femoral nerve 102 lies in the path 826 such that the RF energy can modulate the articular branch 104. The stylet 612 can be retracted back into the needle body 602. After modulating the articular branch nerve 104, the pain is reduced.

Using a ventral and caudal approach, user can insert a treatment device at a seventh point 832. Other approaches are also possible. In some embodiments, the limb of the hip joint may be lifted to increase access. The user can advance the treatment device until the distal end contacts bone. Facets of the hip joint capsule, the obturator foramen, the acetabular notch, blood vessels, and/or other anatomical landmarks may be used to help determine a position of the seventh point 832. If the treatment device comprises the treatment device 600, for example, imaging can help to orient the treatment device 600 so that the stylet 612 is deployed until the eighth point 834. Facets of the hip joint capsule, blood vessels, and/or other anatomical landmarks may be used to help determine a position of the eighth point 834. Imaging (e.g., fluoroscopy, ultrasound, etc.) may be used to verify the position of the treatment device prior to modulation. Bipolar radiofrequency energy can be applied between the first electrode 604 and the second electrode 614. The energy will extend along the path 836, forming a substantially linear or cylindrical ablation zone. The articular branch 134 from the obturator nerve 132 lies in the path 836 such that the RF energy can modulate the articular branch 134. The stylet 612 can be retracted back into the needle body 602. After modulating the articular branch nerve 134, the pain is reduced.

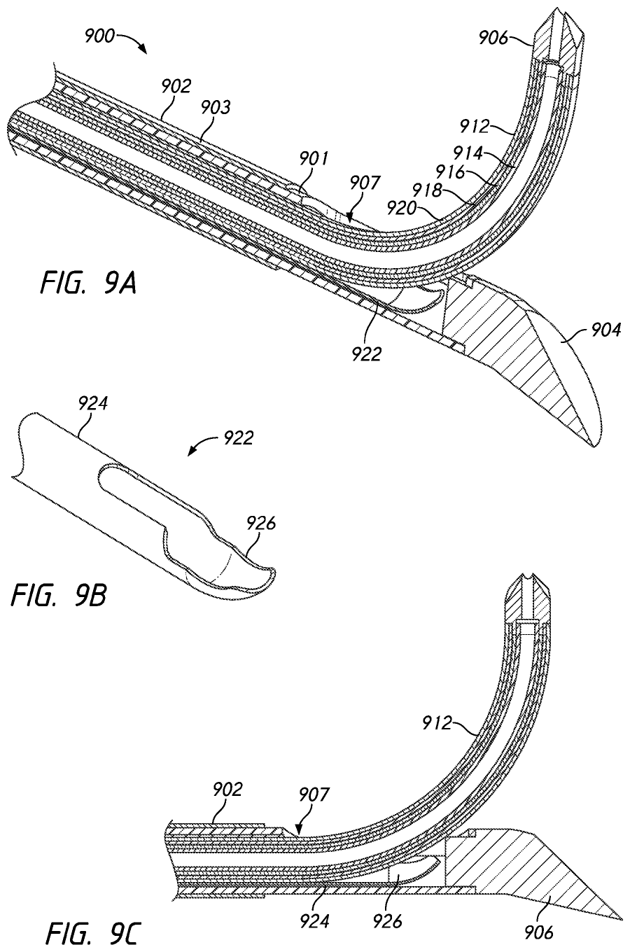

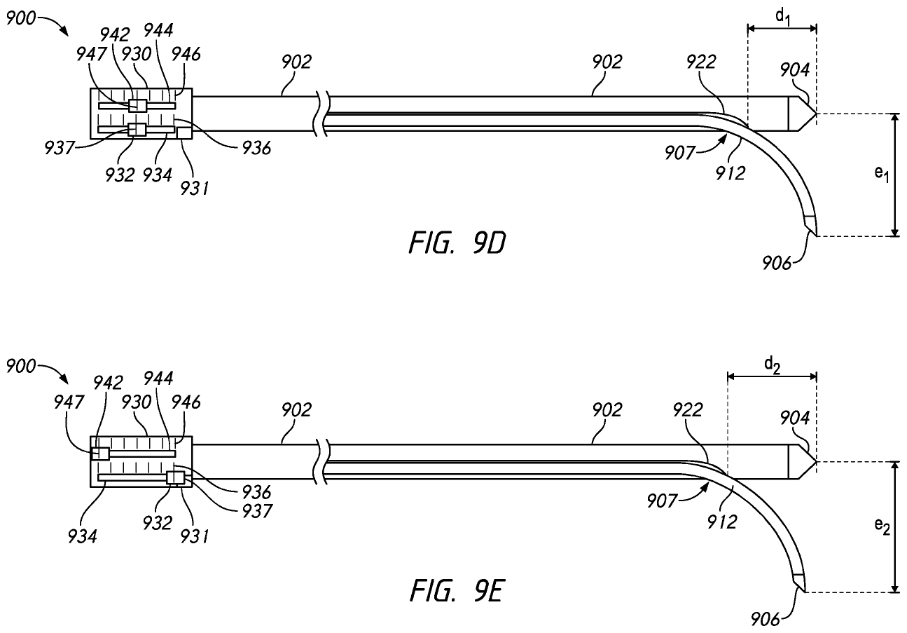

FIG. 9A is a partial cross-sectional perspective view of an example tissue treatment device 900. The device 900 may share features with the device 600 and/or other tissue treatment devices herein (e.g., radiopaque markers, echogenic surfaces, electrode type, tip type, sensors, materials, dimensions including distances, usage, handle features, connection to a tissue treatment (e.g., nerve modulation (e.g., ablation)) system, etc.). The treatment device 900 comprises a needle body 902. The needle body 902 may comprise a shaft 901 and an outer tube 903. The outer tube 903 may comprise a heat shrink material. The distal end of the needle body 902 comprises a tissue-penetrating tip 904. The tip 904 comprises a first electrode. The portion of the needle body 902 proximal to the tip 904 may comprise electrically insulating material. The needle body 902 comprises an aperture 907. The needle body 902 may comprise a longitudinally-extending lumen in communication with the aperture 907. The needle body 902 may be hollow at least proximal to the aperture 907.

The treatment device 900, in some embodiments, comprises a stylet 912 configured to exit the aperture 907 and away (e.g., curve radially away) from the needle body 902. In one embodiment, the aperture 907 is located on a lateral surface of the needle body 902. The needle body 902 may comprise a longitudinally-extending lumen in communication with the aperture 907. The needle body 902 may be hollow at least proximal to the aperture 907. The treatment device 900 comprises a stylet 912 configured to exit the aperture 907 and curve radially away from the needle body 902. The treatment device 900 comprises a stylet 912 configured to exit the lateral aperture 907 and extend laterally away from the needle body 602 at an angle non-parallel to a longitudinal axis of the needle body 902. In various embodiments, the stylet 912 extends laterally away from the needle body at an angle of 20-160 degrees (e.g., 30°-140°, 45°-135°, 60°-120°, 80°-110°, 20°, 25°, 30°, 35°, 40°, 45°, 50°, 55°, 60°, 65°, 70°, 75°, 80°, 85°, 90°, 95°, 100°, 110°, 115°, 120°, 125°, 130°, 135°, 140°, 145°, 150°, 155°, 160°, and any range or value of angles therein). In one embodiment, the stylet 912 extends with a curve such that the distal tip of the stylet extends perpendicularly from the needle body 902. The aperture 907 may be free of or lack a ramped surface. In one embodiment, the aperture 907 comprises a ramped surface over which the stylet is configured to slide in order to extend at an angle laterally or radially away from the needle body 902. In one embodiment, the ramped surface proximate the aperture 907 is adjustable (e.g., with actuatable with a knob or lever or button) to controllably change the angle of lateral deployment extending the stylet 912 from the needle body 902.

The stylet 912 may comprise a plurality of layers or tubes. For example, the stylet 912 shown in FIG. 9A includes a first tube 914, a second tube 916, a third tube 918, and a fourth tube 920. The first tube 914 may comprise a shape set shape memory tube, for example configured to take a curved shape when not confined by the needle body 902. The second tube 916 may comprise a heat shrink. The third tube 918 may comprise a shape set and laser cut shape memory tube, for example configured to take a curved shape when not confined by the needle body 902. The third tube 918 in combination with the first tube 914 can provide refinement of the shape, additional stiffness, and/or a secondary pathway for return if desired. The fourth tube 920 may comprise a heat shrink. For example, the fourth tube may at least partially fill or cover features cut into the third tube 918. The distal end of the stylet 912 comprises a tissue-penetrating tip 906. The tip 906 comprises a second electrode. More or fewer tubes are also possible. For example, the first tube 914 or the third tube 918 may be omitted. The second electrode may be electrically coupled to a signal generator by, for example, the first tube 914 and/or the third tube 918. Bipolar radiofrequency signals can be applied between the first electrode and the second electrode. In some embodiments, the first electrode and the second electrode can be separate from the tips 904, 906, for example as described with respect to the device 600. In various embodiments, the device may have enhanced/elevated stiffness, column strength, bending moment, hardness to account for increased tissue depth, length, musculature, density, of the breed or species of the patient.

The device 900 comprises a ramp 922. FIG. 9B is a partial perspective view of an example component, specifically the ramp 922, of the tissue treatment device 900 of FIG. 9A. FIG. 9C is a partial cross-sectional side view of the tissue treatment device 900 of FIG. 9A. The ramp 922 comprises an elongate section 924 and a deflection section 926. The elongate section 924 is coupled to an actuator in a handle that is configured to longitudinally move the ramp 922. The elongate section 924 may comprise a tube (e.g., as shown in FIG. 9B), a wire, a flat segment, etc. The deflection section 926 is configured to direct the stylet 912 laterally out of the aperture 907 of the needle body 902. The deflection section 926 may be curved (e.g., comprising a continuous curve as shown in FIGS. 9A-9C, comprising a plurality of arcuate segments), straight (e.g., at an angle), combinations thereof, and the like. The ramp 922 is longitudinally movable in the needle body 902.

FIG. 9D is a schematic diagram of the tissue treatment device 900 of FIG. 9A. The device 900 comprises a handle 930. The handle 930 may share features with the handle 630 (e.g., electronic circuitry 931, gearing, etc.). The handle 930 comprises a first knob 932 configured to slide in a first channel 934 and a second knob 944 configured to slide in a second channel 944. Distally advancing the second knob 942 distally advances the stylet 912. The handle 930 comprises indicia 946, for example alignable with indicia 947 on the knob 942, to provide a user with information about the extent of extension of the stylet 912. Distally retracting the first knob 932 distally retracts the ramp 922. The handle 930 comprises indicia 936, for example alignable with indicia 937 on the knob 932, to provide a user with information about the extent of retraction and/or longitudinal position of the ramp 922. In some implementations, the channel 934 approximates the aperture 907 (e.g., the position of the knob 932 in the channel 934 approximates the position of the ramp 922 in the aperture 907).

The position of the ramp 922 and the extent of deployment of the stylet 912 can influence the position of the second electrode of the stylet 912 (e.g., the tip 906 or a separate electrode structure). For example, in FIG. 9D, the knob 942 is retracted about half way through the channel 944 such that the distal tip of the ramp 922 is a first distance di from the distal tip of the tip 904, and the knob 932 is advanced about half way through the channel 934 such that the distal tip of the tip 906 of the stylet 912 is a first lateral distance e₁ from the distal tip of the tip 904, the longitudinal axis of the needle body 902, or the like. Applying a bipolar radiofrequency signal between the tip 904 and tip 906 will create an approximately linear lesion between the tip 904 and the tip 906. If the tip 904 is proximate to a bone and the tip 906 is in the position illustrated in FIG. 9D, the lesion will be proximate to the bone.

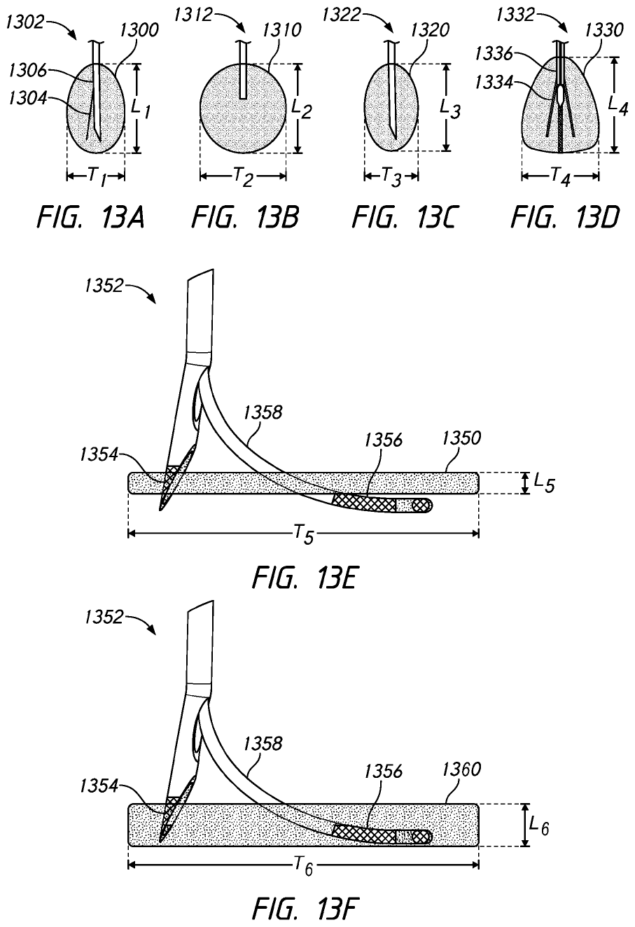

FIG. 9E is another schematic diagram of the tissue treatment device 900 of FIG. 9A. In some embodiments, the user may desire to take a different angle of deployment of the stylet 912, for example due to the tissue around the treatment site. Adjusting where the stylet 912 comes out the side of the needle body 902 can affect the size of the ablation (e.g. thickness T in FIG. 13E). For example, if the stylet 912 comes out more proximally, the thickness T will be relatively larger, and if the stylet 912 comes out more distally, the thickness T will be relatively smaller. Adjusting where the stylet 912 comes out of the side of the needle body 902 can ensure that the second electrode makes it around the curvature of the joint surface. For example, if the needle body 902 is docked on the dorsal aspect (12:00) on the acetabular rim (e.g. dorsal rim), the surface is concave. The position of the ramp 922 and the extent of deployment of the stylet 912 can again influence the position of the second electrode of the stylet 912 (e.g., the tip 906 or a separate electrode structure). For example, in FIG. 9E, the knob 942 is fully retracted through the channel 944 such that the distal tip of the ramp 922 is a second distance d₂ (greater than the first distance d₁) from the distal tip of the tip 904, and the knob 932 is fully advanced through the channel 934 such that the distal tip of the tip 906 of the stylet 912 is a second lateral distance e₂ (greater than the first lateral distance e₁) from the distal tip of the tip 904, the longitudinal axis of the needle body 902, or the like. Even though the stylet 912 is advanced from the needle body 902 a greater longitudinal distance and a greater lateral distance, the tip 906 is still proximate a same longitudinal position as the tip 904, which can both be proximate a bone, for example.

In some embodiments, the knobs 932, 942 can be mechanically linked. For example, if the treatment method results in the tip 906 being proximate a same longitudinal position as the tip 904, the handle 930 can include gears, stop surfaces, etc. such that proximal retraction of the knob 942 affects the permitted extent of advancement of the knob 932. In some implementations, the indicia 936, 946 can be color coded. For example, if the ramp 922 is in a proximal-most position, then the stylet 912 is fully advanced out of the aperture 907, so the proximal-most indicia 946 and the distal-most indicia 936 can be the same color to alert the user that matching colors means proper positioning.

The ramp 922 moves longitudinally back and forth. For example, in some embodiments, the ramp 922 does not move laterally (e.g., out of the needle body 902). The ramp 922 is rigid or otherwise maintains its shape. For example, in some embodiments, the deflection surface 926 does not deform, tilt, etc. to change an angle of deflection. The ramp 922 comprises an open deflection surface. For example, in some embodiments, the deflection surface 926 does not comprise a fully annular tube. The ramp 922 is in the needle body 902. For example, in some embodiments, the device 900 does not comprise a tubular that interacts with the needle body 902 to change the aperture 907 or the like.

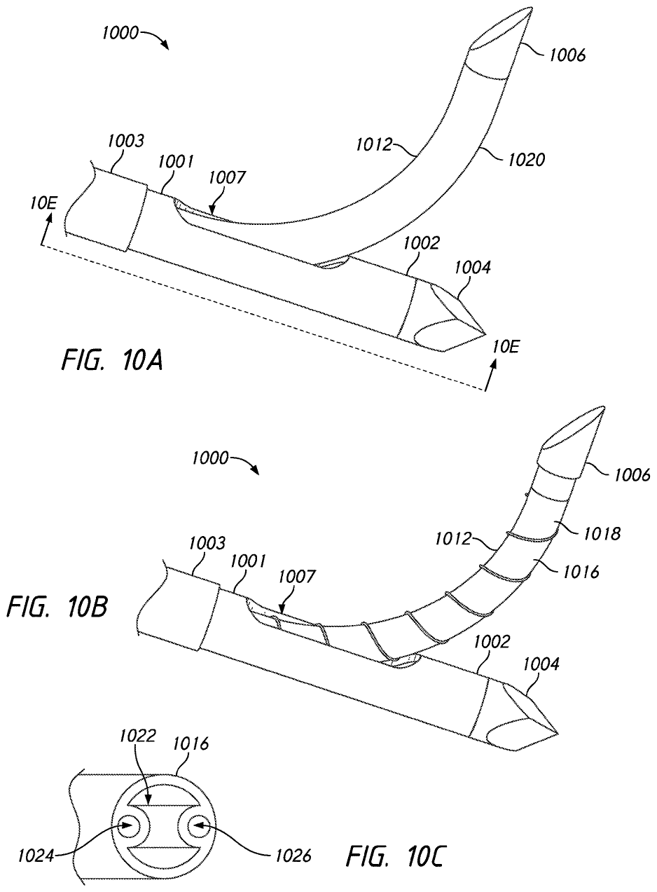

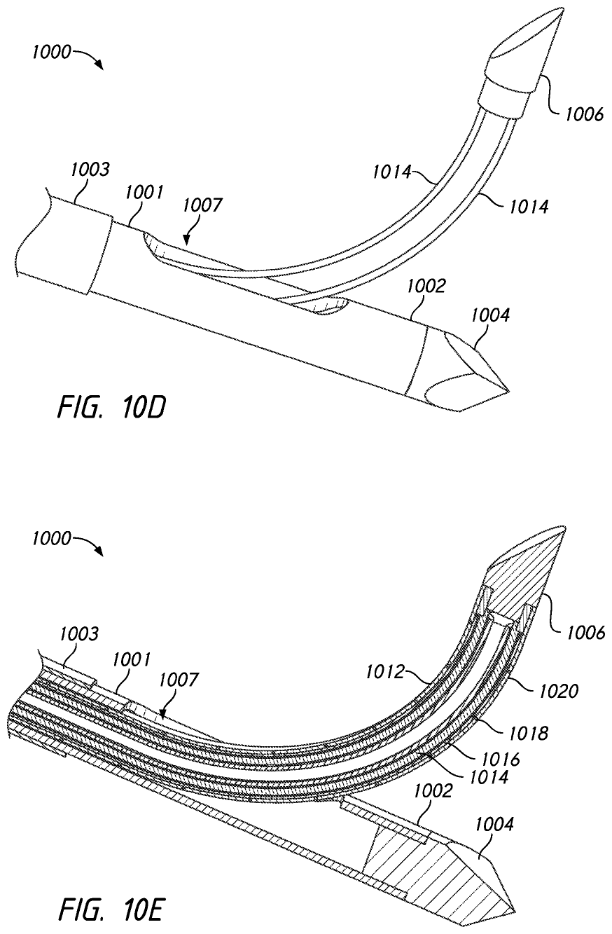

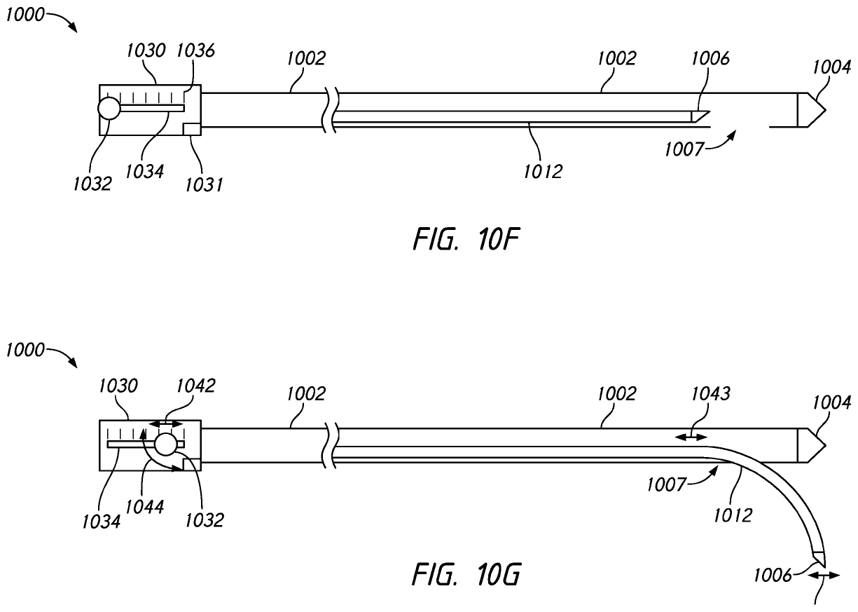

FIG. 10A is a partial perspective view of an example tissue treatment device 1000. The device 1000 may share features with the device 600, the device 900, and/or other tissue treatment devices herein (e.g., radiopaque markers, echogenic surfaces, electrode type, tip type, sensors, materials, dimensions including distances, usage, handle features, connection to a tissue treatment (e.g., nerve modulation (e.g., ablation)) system, movable ramp, etc.). The treatment device 1000 comprises a needle body 1002. The needle body 1002 may comprise a shaft 1001 and an outer tube 1003. The outer tube 1003 may comprise a heat shrink material. The distal end of the needle body 1002 comprises a tissue-penetrating tip 1004. The tip 1004 comprises a first electrode. The portion of the needle body 1002 proximal to the tip 1004 may comprise electrically insulating material. The needle body 1002 comprises an aperture 1007. The needle body 1002 may comprise a longitudinally-extending lumen in communication with the aperture 1007. The needle body 1002 may be hollow at least proximal to the aperture 1007.

The treatment device 1000 comprises a stylet 1012 configured to exit the aperture 1007 and curve radially away from the needle body 1002. The stylet 1012 may comprise a plurality of layers or tubes or components. For example, FIG. 10A shows the outer tube 1020 of the stylet 1012. The outer tube 1020 may comprise a heat shrink. The outer tube 1020 may comprise a polymer such as polyether ether ketone (PEEK), polytetrafluoroethylene (PTFE), combinations thereof, or the like. The distal end of the stylet 1012 comprises a tissue-penetrating tip 1006. The tip 1006 comprises a second electrode.

FIG. 10B is a partial perspective view of the tissue treatment device 1000 of FIG. 10A with a component, specifically the stylet outer tube 1020, removed. FIG. 10B shows a catheter 1016 and an optional stiffener 1018. The stiffener 1018 may comprise a coil (e.g., as shown in FIG. 10B), a braid, a tube, combinations thereof, or the like. FIG. 10C is a partial cross-sectional distal end view of an example component, specifically the catheter 1016, of the tissue treatment device 1000 of FIG. 10A. The catheter 1016 comprises a first lumen 1024 configured to house a pull wire. The catheter 1016 optionally comprises a second lumen 1026 configured to house a second pull wire. The first lumen 1024 may be circumferentially opposite (e.g., circumferentially spaced by 180°) the second lumen 1026. The first lumen 1024 may have a diameter between about 0.005 inches (in.) (approx. 0.13 mm) and about 0.02 in. (approx. 0.51 mm) (e.g., about 0.005 in. (approx. 0.13 mm), about 0.008 in. (approx. 0.2 mm), about 0.011 in. (approx. 0.28 mm), about 0.014 in. (approx. 0.36 mm), about 0.017 in. (approx. 0.43 mm), about 0.02 in. (approx. 0.51 mm), ranges between such values, and the like). The catheter 1016 optionally comprises a central lumen 1022. The central lumen 1022 may take a shape remaining after the lumen 1024 and optionally the lumen 1026 are removed (e.g., as shown in FIG. 10C). The central lumen 1022 may have a circular, oval, elliptical, etc. lateral cross section. The catheter 1016 may comprise a polymer such as PEEK, PTFE, combinations thereof, or the like. The catheter 1016 may have an outer diameter between about 2 French (Fr) (approx. 0.026 in.; approx. 0.66 mm) and about 6 Fr (approx. 0.078 in.; approx. 2 mm) (e.g., about 2 Fr (approx. 0.026 in.; approx. 0.66 mm), about 3 Fr (approx. 0.039 in.; approx. 1 mm), about 4 Fr (approx. 0.052 in.; approx. 1.3 mm), about 5 Fr (approx. 0.065 in.; approx. 1.6 mm), about 6 Fr (approx. 0.078 in.; approx. 2 mm), ranges between such values, and the like). The catheter 1016 may have an outer diameter between 22-14 Gauge (e.g., 16-18, 14-20 Gauge, 22, 21, 20, 19, 18, 17, 16, 15, 14 Gauge and other values and ranges therein).

FIG. 10D is a partial perspective view of the tissue treatment device 1000 of FIG. 10A with additional components, specifically the stiffener 1018 and the catheter 1016, removed. FIG. 10D shows pull wires 1014. The pull wires 1014 extend through the lumens 1024, 1026. The pull wires 1014 are configured to deflect the stylet 1012 proximally and distally. For example, if the pull wire 1014 connected to an upper side of the tip 1004 (left as illustrated in FIG. 10D) is proximally retracted, the tip 1006 will deflect proximally, and if the pull wire 1014 connected to a lower side of the tip 1004 (right as illustrated in FIG. 10D) is proximally retracted, the tip 1006 will deflect distally. In one embodiment, the stylet 1012 only deflects in one or two directions along a plane including the longitudinal axis of the needle body 1002. One or both of the pull wires 1014 can provide electrical signals to the tip 1006. In some embodiments, a separate wire or other electrical conductor (e.g., extending through the central lumen 1022) provides electrical signals to the tip 1006.

FIG. 10E is a partial cross-sectional side view of the tissue treatment device 1000 of FIG. 10A along the line 10E-10E. FIG. 10E shows the components of the stylet 1012 in combination. For example, FIG. 10E shows the pull wires 1014 extending through the lumens 1024, 1026 of the catheter 1016 and coupled to the tip 1006.

FIG. 10F is a schematic diagram of the tissue treatment device 1000 of FIG. 10A. The device 1000 comprises a handle 1030. The handle 1030 may share features with the handle 630, 930 (e.g., electronic circuitry 1031, gearing, etc.). The handle 1030 comprises a knob 1032 configured to slide in a channel 1034 and to rotate about an axis perpendicular to the channel 1034. Distally advancing the knob 1032 distally advances the stylet 1012. In one embodiment, treatment via activation of the electrodes in a tissue treatment device 1000 is performed with the stylet 1012 in a non-extended position, e.g., with the electrode(s) of the stylet within the aperture 1007. In one embodiment, treatment via activation of the electrodes in a tissue treatment device 1000 is performed with the stylet 1012 in a minimally-extended position, e.g., with the electrode(s) of the stylet extending just outside the aperture 1007. In various embodiments, treatment via activation of the electrodes in a tissue treatment device 1000 is performed with the stylet 1012 in an extended position, e.g., with the electrode(s) of the stylet extended at one or more fixed and/or adjustable lengths from the aperture 1007. The handle 1030 comprises indicia 1036 to provide a user with information about the extent of extension of the stylet 1012. Rotating the knob 1032 in a first direction (e.g., clockwise) deflects the stylet 1012 in a first direction (e.g., proximally) and rotating the knob 1032 in a second direction (e.g., counterclockwise) deflects the stylet 1012 in a second direction (e.g., distally). By rotating the knob 1032 and moving the knob 1032 through the channel 1034, a user can effectively steer the stylet 1012. Steering the stylet 1012 can help to make sure that the tip 1006 is close to bone and does not deflect upwardly into muscle, for example, upon additional extension due in contrast to a stylet having an accurate shape set that might begin to curve away from bone as the stylet is further extended. In some anatomy, the stylet 1012 may desirable deflect distally to be able to remain close to bone. Steering the stylet 1012 can help to avoid crucial structures. Steering the stylet 1012 can allow for poor initial placement or variances in anatomy from patient to patient (e.g., initial placement may be the same for every case, but variances in anatomy such as joint shape, size, etc. and whether congenital or due to disease may use a change in direction to navigate to desired end point. The acetabulum and most joint surfaces are curved/concave. The adjustability provided by a steerable stylet 1012 can allow a user to articulate/steer the electrode around curved structures. In various embodiments, adjustability and steerability is enhances with elevated strength, torque, column strength, rigidity, hardness, and/or other characteristics to account for increased tissue depth, length, density, musculature, ligaments, joint capsule structure of the breed and/or species of the patient. Steering the stylet 1012 can allow the tip 1006 to be navigated to a desired position that the stylet 1012 may not otherwise achieve upon deployment. For example, the tip 1006 may be deflected to traverse along a bone for a desired extension distance, whereas a non-steered stylet may start to curve away from bone after some extension distance. Steering the stylet 1012 can allow the stylet 1012 to avoid certain tissue. For example, the stylet may be deflected to avoid tendons, cartilage, etc. that a non-steered stylet may damage.

FIG. 10G is another schematic diagram of the tissue treatment device 1000 of FIG. 10A. FIG. 10G shows the knob 1032 moving through the channel 1034, as indicated by the arrow 1042 and the corresponding arrow 1043, and being rotated, as shown by the arrow 1044 and the corresponding arrow 1045, which shows the deflection upon rotation of the knob 1032. Other mechanisms are also possible. For example, the knob 1032 could comprise an annular element extending around the handle 1030. For another example, the knob 1032 could comprise a lever configured to tilt proximally and distally to deflect the stylet 1012 proximally and distally.

In some embodiments, the knob 1032 can be mechanically geared to couple the rotation and the position along the channel 1034. For example, the knob 1032 may be configured so that rotation of the knob 1032 has a greater effect when the knob 1032 is further distally advanced than when the knob 1032 is less distally advanced.

In one embodiment, the steerable stylet 1012 extends out of a lateral side of the needle body 1002. For example, in some embodiments, the stylet 1012 does not exit or extend out of a distal end of the needle body 1002. The steerable stylet 1012 is configured to navigate through soft tissue. For example, in some embodiments, the stylet 1012 cannot penetrate bone or navigate through spongy bone. The steerable stylet 1012 can comprise one, two, three or more electrodes that are used for neuromodulation (e.g., ablation), and thus should not be confused with a guidewire, a guide catheter, etc. In some embodiments, the steerable stylet 1012 is not a hollow steerable component that is then used to guide a separate electrode-bearing tube.

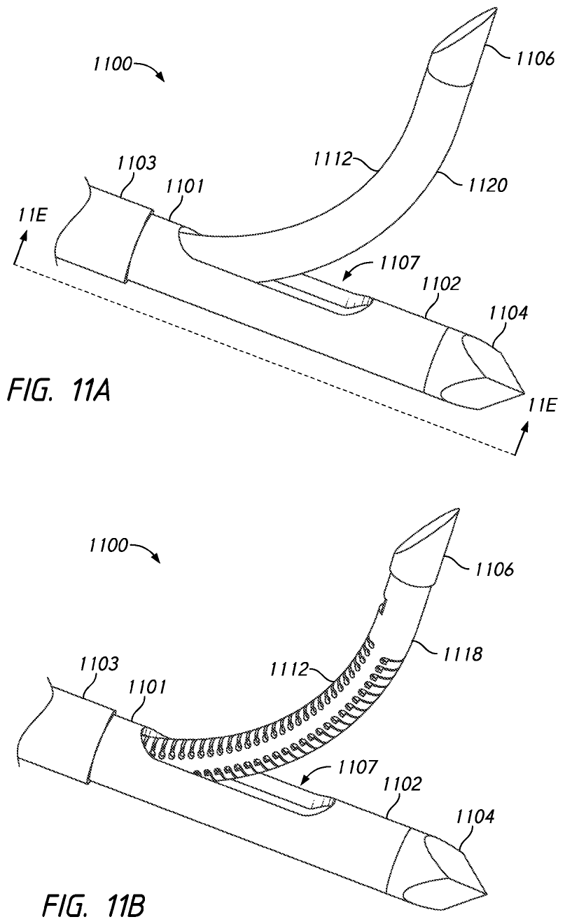

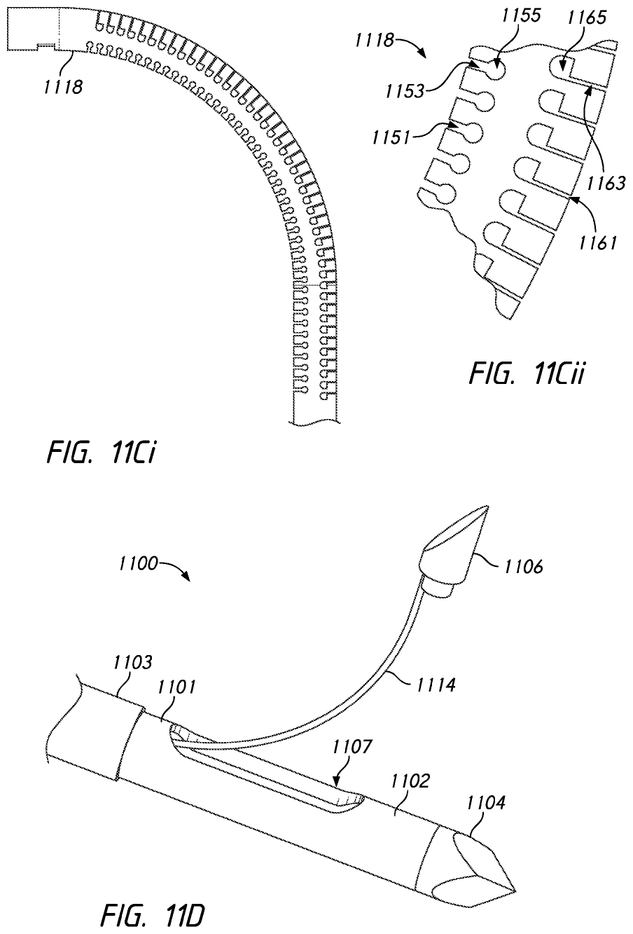

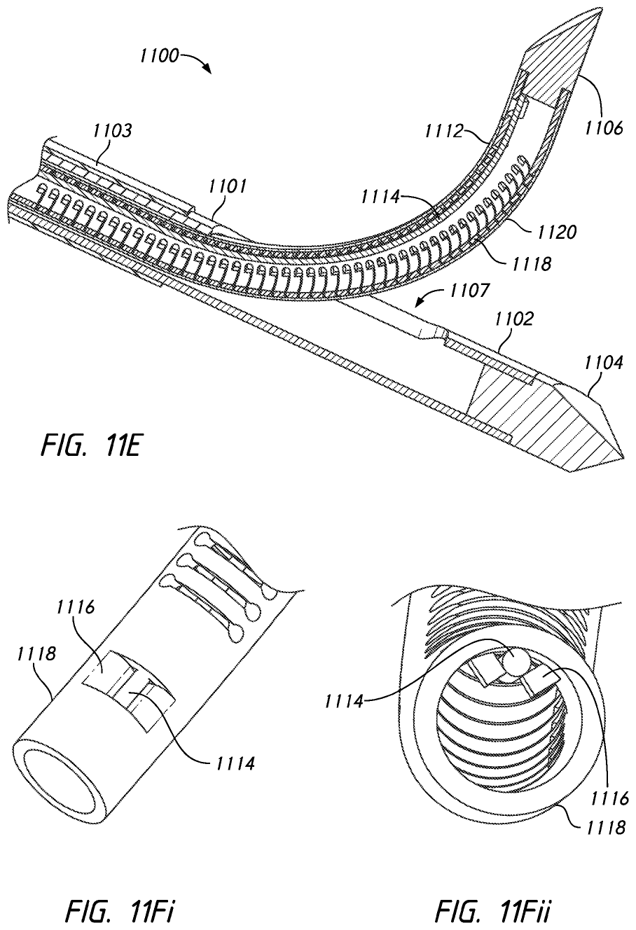

FIG. 11A is a partial perspective view of an example tissue treatment device 1100. The device 1100 may share features with the device 600, the device 900, the device 1000, and/or other tissue treatment devices herein (e.g., radiopaque markers, echogenic surfaces, electrode type, tip type, sensors, materials, dimensions including distances, usage, handle features, connection to a tissue treatment (e.g., nerve modulation (e.g., ablation)) system, movable ramp, etc.). The treatment device 1100 comprises a needle body 1102. The needle body 1102 may comprise a shaft 1101 and an outer tube 1103. The outer tube 1103 may comprise a heat shrink material. The distal end of the needle body 1102 comprises a tissue-penetrating tip 1104. The tip 1104 comprises a first electrode. The portion of the needle body 1102 proximal to the tip 1104 may comprise electrically insulating material. The needle body 1102 comprises an aperture 1107. The needle body 1102 may comprise a longitudinally-extending lumen in communication with the aperture 1107. The needle body 1102 may be hollow at least proximal to the aperture 1107.

The treatment device 1100 comprises a stylet 1112 configured to exit the aperture 1107 and curve radially away from the needle body 1102. The stylet 1112 may comprise a plurality of layers or tubes or components. For example, FIG. 11A shows the outer tube 1120 of the stylet 1112. The outer tube 1120 may comprise a heat shrink. The outer tube 1120 may comprise a polymer such PEEK, PTFE, combinations thereof, or the like. The outer tube 1120 may reduce damage to the needle body 1102 (e.g., from friction between the stylet 1112 and the needle body 1102 during deployment and retraction of the stylet 1112). The outer tube 1120 may have a thickness, for example, between about 0.002 in (approx. 0.05 mm) and about 0.02 in. (approx. 0.5 mm) (e.g., about 0.002 in (approx. 0.05 mm), about 0.005 in (approx. 0.13 mm), about 0.008 in (approx. 0.2 mm), about 0.01 in (approx. 0.25 mm), about 0.015 in (approx. 0.38 mm), about 0.02 in (approx. 0.5 mm), ranges between such values, and the like). The distal end of the stylet 1112 comprises a tissue-penetrating tip 1106. The tip 1106 comprises a second electrode.

FIG. 11B is a partial perspective view of the tissue treatment device 1100 of FIG. 11A with a component, specifically the stylet outer tube 1120, removed. FIG. 11B shows that the stylet 1112 comprises a tube 1118. The tube 1118 may comprise a coil, a braid, a tube, combinations thereof, or the like. The tube 1118 shown in FIG. 11B comprises a patterned (e.g., laser-cut) hypotube. The tube 1118 may be shape set (e.g., to a particular curvature). The tube 1118 may be not shape set. The tube 1118 may comprise, for example, stainless steel or other biocompatible materials. In some embodiments, the tube 1118 does not comprise a shape memory material such as nitinol.

FIG. 11Ci is a side view of an example component, the tube 1118, of the tissue treatment device 1100 of FIG. 11A. FIG. 11Cii is an expanded side view of the example component, the tube 1118, of FIG. 11Ci. The tube 1118 comprises a first plurality of kerfs 1151 and a second plurality of kerfs 1161. The first plurality of kerfs 1151 are on a first circumferential side of the tube 1118 and the second plurality of kerfs 1161 are on a second side of the tube 1118 opposite the first plurality of kerfs 1151. The pluralities of kerfs 1151, 1161 allow the tube 1118 to bend in one or two directions along a plane (e.g., the plane of the page with respect to FIG. 11Ci). The tube 1118, in one embodiment, does not bend in another direction during normal use of the device 1100. The kerfs 1151 may be the same as the kerfs 1161.

The kerfs 1151 may be different than the kerfs 1161 (e.g., as shown in FIG. 11Ci and 11Cii). The kerfs 1151 may comprise a straight portion 1153 and an expanded portion 1155. The kerfs 1161 may comprise a straight portion 1163 and an expanded portion 1165. The straight portions 1153 may be thicker than the straight portions 1163. The straight portions 1153 may be shorter than the straight portions 1163. The expanded portions 1155 may be larger in area than the expanded portions 1165. The expanded portions 1155 may be a different shape than the expanded portions 1165. For example, FIG. 11Cii shows the expanded portions 1155 being circular and centered on the straight portions 1153, and the expanded portions 1165 being semicircular and having edges aligned with the straight portions 1151. The kerfs 1151 may have a first spacing and the kerfs 1161 may have a second spacing. The second spacing may be greater that the first spacing.

The differences in the kerfs 1151, 1161 can influence the bending of the tube 1118. For example, when the tube 1118 bends towards the kerfs 1151 (e.g., as shown in FIG. 11Cii), the lack of material in the wide straight portions 1153 can allow material to take the place of the space. Conversely, if the tube 1118 were to bend in the opposite direction, the lack of material in the narrow straight portions 1163 would allow less material to take the place of the space and impart less bending. For another example, when the tube 1118 bends towards the kerfs 1151 (e.g., as shown in FIG. 11Cii), the expanded portions 1155 can provide pivot points around along the same longitudinal axis as the straight portions 1153, and the expanded portions 1165 can provide pivot points that are on a different longitudinal axis as the straight portions 1163. These shapes and/or differentials can help to spread out the load and produce a device that is less prone to cracking and/or longer lasting. For another example, when the tube 1118 bends towards the kerfs 1151 (e.g., as shown in FIG. 11Cii), the kerfs 1151 being closely spaced can provide a reduced radius of curvature and the kerfs 1161 being more widely spaced can provide a higher radius of curvature, as desired for the bend.

The kerfs 1151, 1161 may be consistent over the substantial length of the tube 1118 (e.g., as shown in FIG. 11Ci). The kerfs 1151, 1161 may be vary over the length of the tube 1118 (e.g., having variable spacing, dimensions, and/or shapes, for example to make the tube 1118 more flexible towards the distal end). Other cut patterns are also possible. For example, the kerfs 1151, 1161 can be straight lines, I-lines, dumbbell lines, etc. For another example, kerfs can be formed from an interrupted helix. The outer tube 1120 may at least partially fill and/or cover kerfs in the tube 1118, for example to avoid pinching, inhibit liquid from flowing through the kerfs, etc. If the tube 1118 is conductive, the outer tube 1120 can electrically insulate the tube 1118.

FIG. 11D is a partial perspective view of the tissue treatment device 1100 of FIG. 11A with another component, specifically the tube 1118, removed, according to some embodiments. FIG. 11D shows a pull wire 1114. The pull wire 1114 extends through a lumen of the tube 1118. The pull wire is connected to a side of the tip 1106. The side is the same side as the kerfs 1151. The pull wire 1114 is configured to deflect the stylet 1112 proximally. For example, if the pull wire 1114 is proximally retracted, the tip 1106 will deflect proximally. In some embodiments, the stylet 1112 only deflects in one or two directions along a plane including the longitudinal axis of the needle body 1102. In some embodiments, the tube 1118 is shape set (e.g., to be straight, to have a curve as shown in FIG. 11D, etc.). In some embodiments, the tube 1118 is not heat treated to impart a shape set, but still reverts towards a straight configuration in the absence of outside forces such as from the pull wire 1114. The pull wire 1114 can provide electrical signals to the tip 1106. In some embodiments, a separate wire, the tube 1118, or other electrical conductor (e.g., extending through the lumen of the tube 1118) provides electrical signals to the tip 1106.

FIG. 11E is a partial cross-sectional side view of the tissue treatment device 1100 of FIG. 11A along the line 11E-11E. FIG. 11E shows the components of the stylet 1112 in combination. For example, FIG. 11E shows the pull wire 1114 extending through the lumen of the tube 1118 and coupled to the tip 1106.

FIG. 11Fi is a partial side and distal end perspective view of example components, specifically the tube 1118 and the pull wire 1114, of the tissue treatment device 1100 of FIG. 11A. FIG. 11Fii is a partial distal end view of the example components, the tube 1118 and the pull wire 1114, of FIG. 11Ei. FIG. 11Fi shows an example implementation of optionally coupling the pull wire 1114 to the tube 1118. A distal segment of the tube 1118 is cut to have a “H” pattern, and then the material 1116 in the H is pressed radially inward. The pull tube 1114 is positioned between the inner surface of the tube 1118 and the formerly outer surface of the tube 1118 that is the pressed H material 1116. The material 1116 has a natural outward spring that bears against the pull wire 1114, holding the pull wire 1114 in place. In some embodiments, the pull wire 1114 may comprise a groove, detent, etc. configured to act with the material 1116. The distal end of the pull wire 1114 in one embodiment is positioned distal to the H material 1116 so that the pull wire 1114 does not prolapse from the tube 1118. Cut shapes other than H can effect a same or similar result (e.g., E, S, U, V, parallel lines, combinations thereof, etc.). In some embodiments, the pull wire 1114 can also or alternatively be coupled to the tube 1118 by welding, soldering, adhesive, combinations thereof, and the like. In some embodiments, the pull wire 1114 is not coupled to the tip 1106 and is only coupled to the tube 1118. In some embodiments, the pull wire 1114 is not coupled to the tube 1118 and is only coupled to the tip 1106.

Coupling the pull tube 1114 to the tube 1118 can enhance manufacturing ease and/or device robustness. For example, the pull wire 1114 can be conductive such that the pull wire 1114 is electrically connected to the tube 1118. The pull wire 1114 and/or the tube 1118 can be electrically connected to the tip 11106 (e.g., as shown in FIG. 11E). Detachment of the pull wire 1114 from the tip 1106, for example, would not inhibit the application of energy during a neuromodulation procedure because the tube 1118 can receive the signals from the pull wire 1114 and transmit those signals to the tip 1106.

In several embodiments, the steerable stylet 1112 extends out of a lateral side of the needle body 1102. For example, in some embodiments, the stylet 1112 does not exit or extend out of a distal end of the needle body 1102. The steerable stylet 1112 is configured to navigate through soft tissue. For example, in some embodiments, the stylet 1112 cannot penetrate bone or navigate through spongy bone. The steerable stylet 1112 includes one, two, three or more electrodes that are used for neuromodulation (e.g., ablation), and thus should not be confused with a guidewire, a guide catheter, etc. In some embodiments, the steerable stylet 1112 is not a hollow steerable component that is then used to guide a separate electrode-bearing tube.

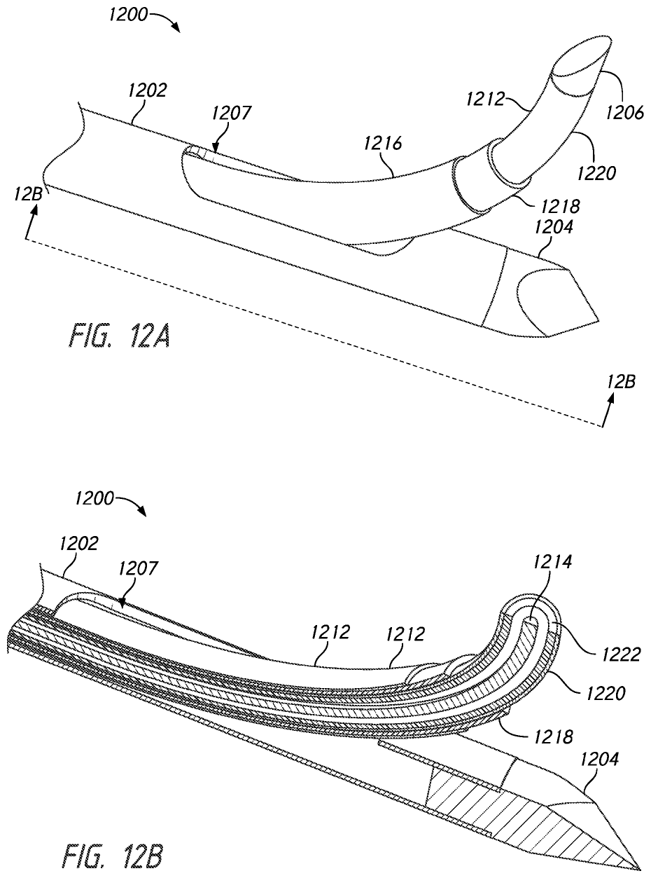

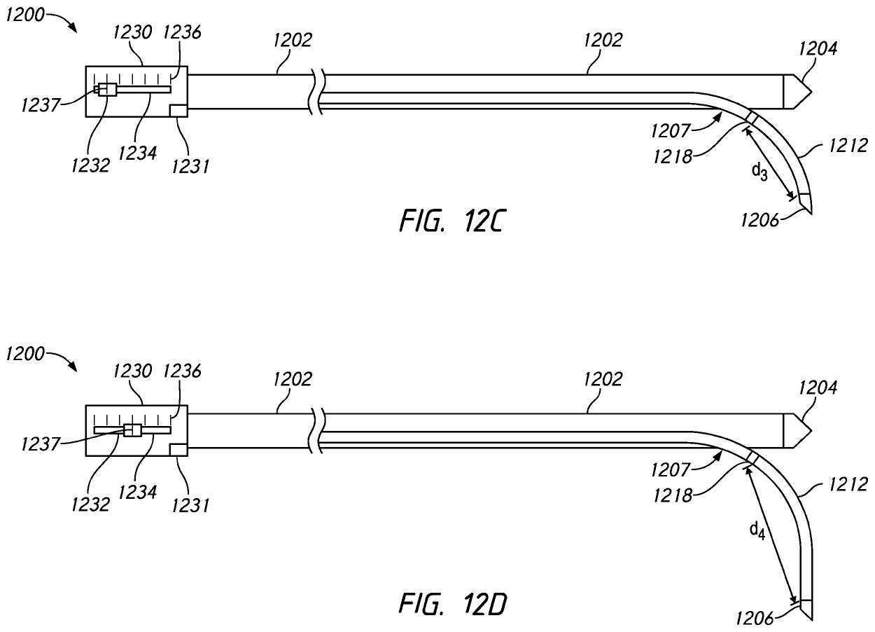

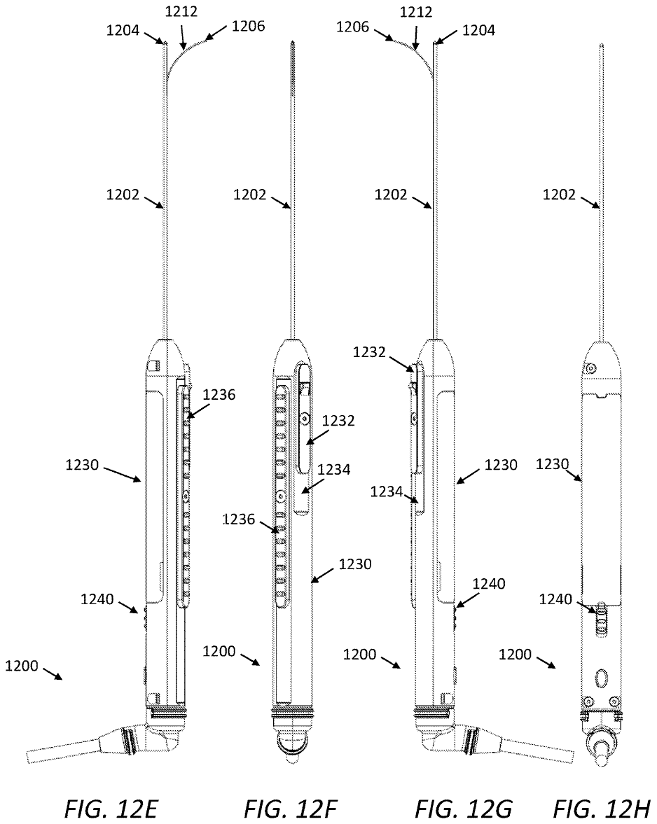

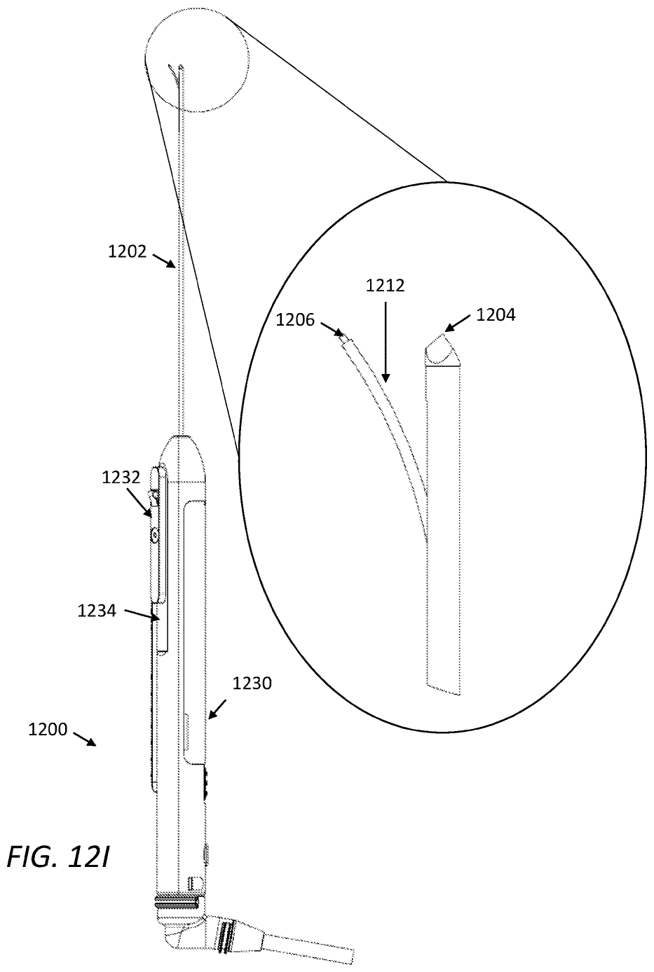

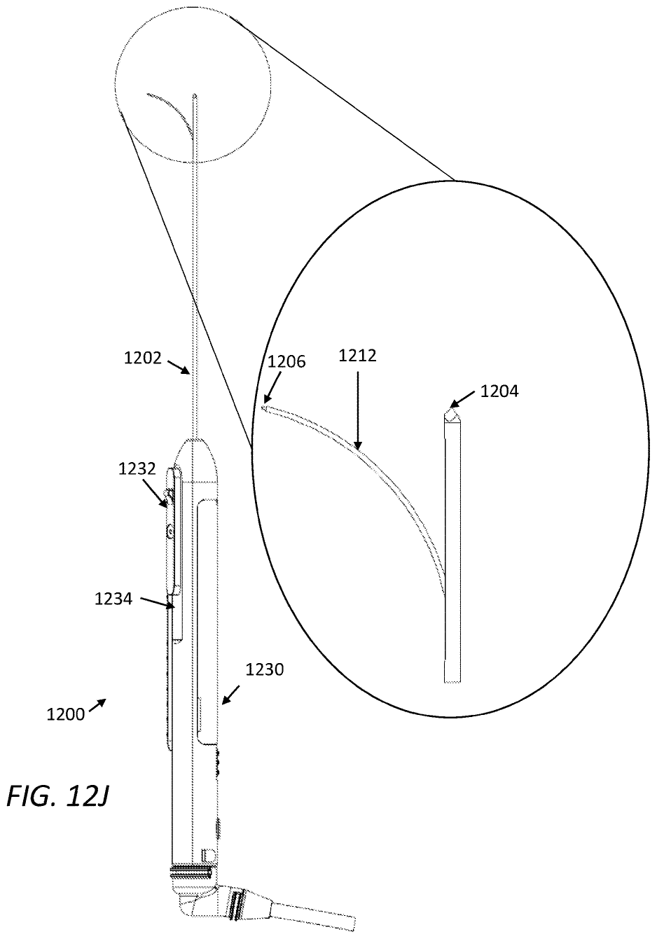

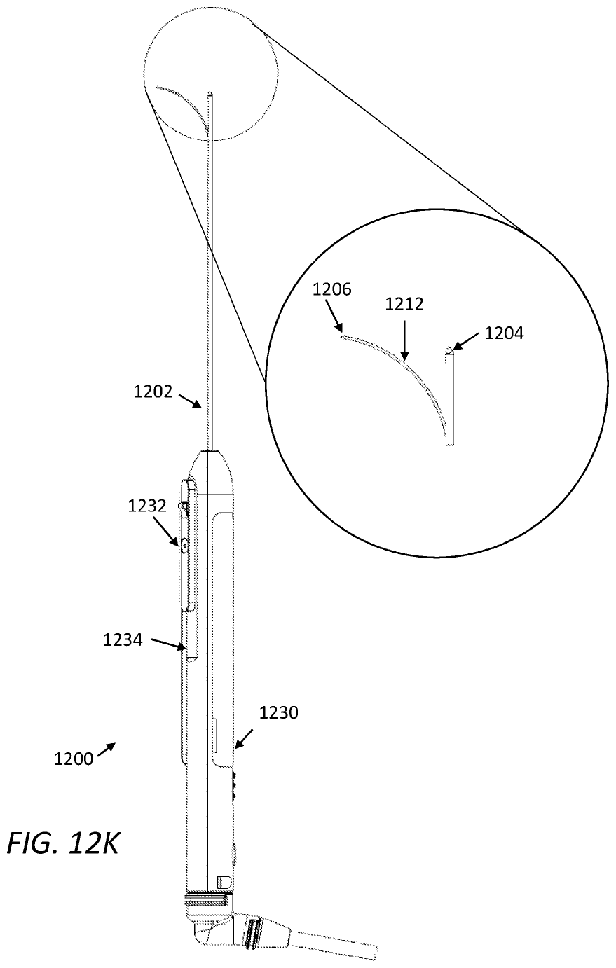

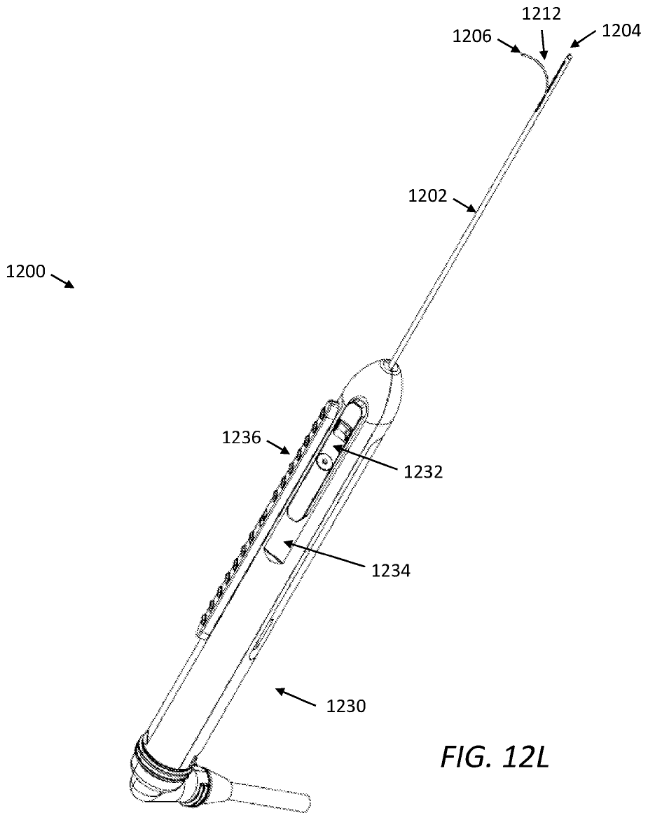

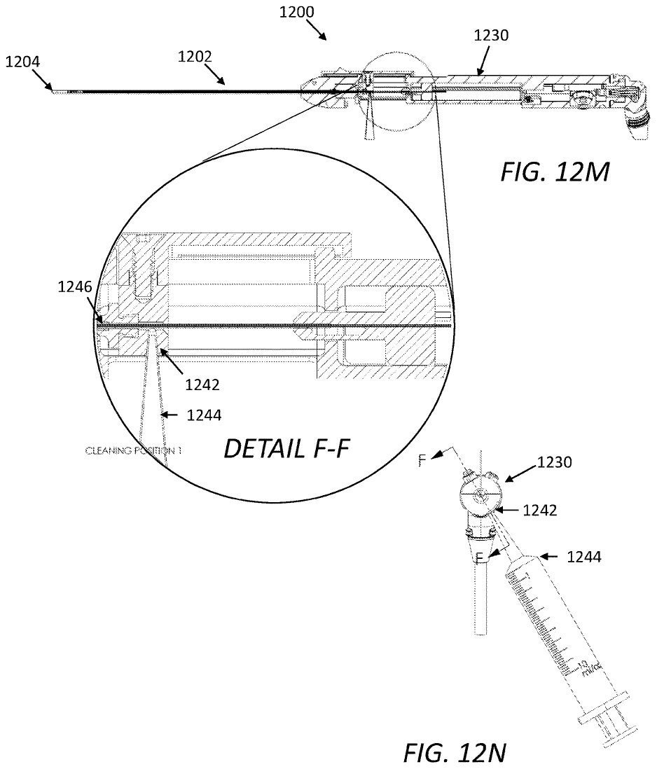

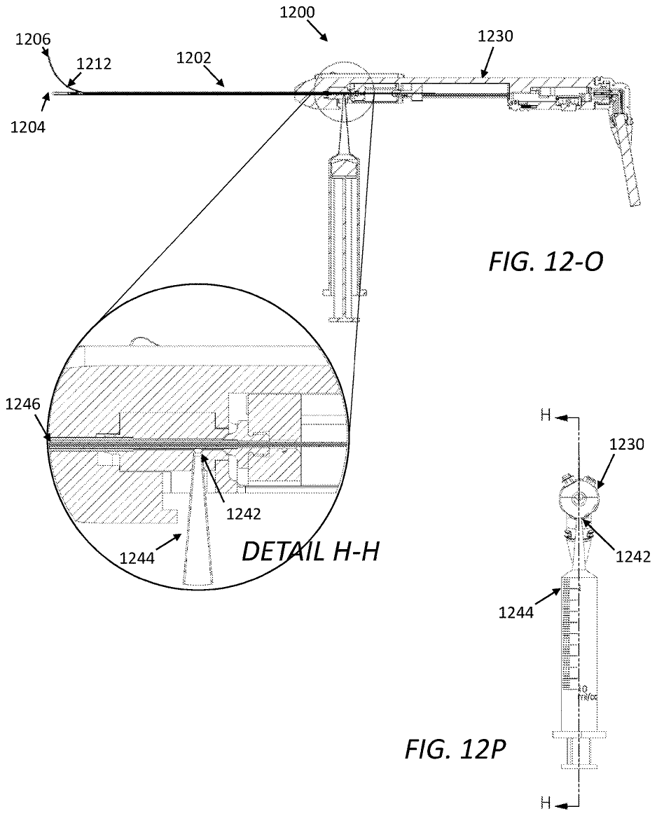

FIG. 12A is a partial perspective view of an example tissue treatment device 1200. The device 1200 may share features with the device 600, the device 900, the device 1000, the device 1100, and/or other tissue treatment devices herein (e.g., radiopaque markers, echogenic surfaces, electrode type, tip type, sensors, materials, dimensions including distances, usage, handle features, connection to a tissue treatment (e.g., nerve modulation (e.g., ablation)) system, movable ramp, etc.). The treatment device 1200 comprises a needle body 1202. The distal end of the needle body 1202 comprises a tissue-penetrating tip 1204. In some embodiments, the tip 1204 may comprises a first electrode. In some embodiments, the tip 1204 and/or the needle body 1202 is free of an electrode. The needle body 1202 comprises an aperture 1207. The needle body 1202 may comprise a longitudinally-extending lumen in communication with the aperture 1207. The needle body 1202 may be hollow at least proximal to the aperture 1207.

The treatment device 1200 comprises a stylet 1212 configured to exit the aperture 1207 and curve radially away from the needle body 1202. The stylet 1212 may comprise a plurality of layers or tubes. For example, the stylet 1212 shown in FIG. 12A includes a first tube 1216, a second tube 1218, and a third tube 1220. The first tube 1216 may comprise an insulating material. The second tube 1218 may comprise a conductive material. The conductive material of the second tube 1218 that is not covered by the first tube 1216 forms a first electrode. The third tube 1220 may comprise an insulating material. The distal end of the stylet 1212 comprises a tissue-penetrating tip 1206. The tip 1206 comprises a second electrode. In some embodiments, the second electrode can be separate from the tip 1206, for example as described with respect to the device 600.

FIG. 12B is a partial cross-sectional side view of the tissue treatment device 1200 of FIG. 12A along the line 12B-12B. FIG. 12B further shows interaction between the tubes 1216, 1218, 1220 and other components. The tip 1206 is removed for clarity. FIG. 12B shows two additional tubes. The stylet 1212 optionally comprises a fourth tube 1222 and a wire 1214. The fourth tube 1222 may, for example, provide column strength during deployment of the stylet 1212 from the needle body 1202. The wire 1214 may comprise a shape set wire (e.g., comprising nitinol) configured to impart curvature to the stylet 1212 when deployed from the needle body 1202. The wire 1214 may be coupled to the tip 1206. The wire 1214 may provide electrical signals to the tip 1206 such that the tip 1206 is the second electrode. The wire 1214 may be electrically insulated from the tube 1218 by the tube 1220 and/or the tube 1222. The tubes 1214, 1216, 1218, 1220, 1222 are generally coaxial about a longitudinal axis.

The tubes 1214, 1220, 1222 telescope relative to the tubes 1216, 1218. When the stylet 1212 is deployed from the needle body 1202, the tubes of the stylet 1212 extend together until the tubes 1216, 1218 reach a stopping point. Upon further deployment, the tubes 1214, 1220, 1222 continue to advance laterally and distally. The tube 1218 comprises a first electrode and the tip 1206 comprises a second electrode such that the distance between the first and second electrodes changes upon additionally deployment of the stylet 1212.

FIG. 12C is a schematic diagram of the tissue treatment device 1200 of FIG. 12A. The device 1200 comprises a handle 1230. The handle 1230 may share features with the handle 630, 930, 1030 (e.g., electronic circuitry 1231, gearing, etc.). The handle 1230 comprises a knob 1232 configured to slide in a channel 1234. Distally advancing the knob 1232 distally advances the stylet 1212. The handle 1230 comprises indicia 1236 to provide a user with information about the extent of extension of the stylet 1212 and/or the distance between the first electrode (e.g., formed from the tube 1218) and the second electrode (e.g., formed from the tip 1206). For example, the indicia 1236 aligned with the knob indicia 1237 in FIG. 12C can correspond to a first distance d₃ between the first electrode and the second electrode.

FIG. 12D is another schematic diagram of the tissue treatment device 1200 of FIG. 12A. FIG. 12D shows the knob 1232 advanced further through the channel 1234. The stylet 1212, in particular the tip 1206, has advanced further out of the needle body 1202, but the position of the tube 1218 is not changed. In some embodiments, the handle 1230 can comprise a detent, indicia, or the like to indicate that the tube 1218 has reached a stopping point. The indicia 1236 aligned with the knob indicia 1237 in FIG. 12D can correspond to a second distance da between the first electrode and the second electrode.

In some embodiments, the first electrode is close enough to the distal end of the device 1200 that an ablation zone between the first electrode and the second electrode can be close to a bone or other surface. The wire 1214 can help the stylet 1212 to take a sharp turn even when advanced out of the needle body 1202 fairly close to the tip 1204. The electrical connections of the device 1200 can all be made at the stylet 1212, which can simplify manufacturing.

The veneered stylet 1212 in some embodiments extends out of one lateral side of the needle body 1202. For example, in some embodiments, the device 1200 does not comprise two stylets exiting or extending out of the needle body 1202. The veneered stylet 1212 can include the first and second electrodes both residing in soft tissue. For example, in some embodiments, the device 1200 does not comprise a first electrode configured to reside in a blood vessel and a second electrode configured to reside in soft tissue.

FIG. 12E is partial side view of the tissue treatment device 1200 of FIG. 12 A. The treatment device 1200 comprises a needle body 1202. The distal end of the needle body 1202 comprises a tissue-penetrating tip 1204. In some embodiments, the tip 1204 may comprises a first electrode. In some embodiments, the tip 1204 and/or the needle body 1202 is free of an electrode. The treatment device 1200 may comprise a stylet 1212 that extends from an aperture of the needle body 1202, as discussed above in conjunction with FIG. 12A. The distal end of the stylet 1212 comprises a tissue-penetrating tip 1206. The tip 1206 comprises a second electrode. In some embodiments, the second electrode can be separate from the tip 1206, for example as described with respect to the device 600. In some embodiments, the stylet 1212 may comprise one or more telescoping tubes.

The device 1200 may include a handle 1230. The handle 1230 may share features with the handle 630, 930, 1030 (e.g., electronic circuitry, gearing, etc.). The handle 1230 comprises indicia 1236 to provide a user with information about the extent of extension of the stylet 1212 and/or the distance between the first electrode (e.g., formed from the a tube of the stylet 1212) and the second electrode (e.g., formed from the tip 1206). The handle 1230 may include a rotatable knob 1240. Gearing (e.g., a worm gear, bevel gears, rack and pinion, etc.) can be used to translate the rotation of the knob 1240 into longitudinal movement of the stylet 1212. In some embodiments, the rotation of the knob 1240 may be translated into a distal/proximal movement of a ramp located at the distal end of the needle body 1202, such as the ramp 922. The handle 1230 may comprise detents (e.g., providing audible and/or tactile feedback), indicia, sensors, etc. to provide a user with information about the extent of extension of the stylet 1212. In some embodiments, the knob 1240 may be longitudinally advanced to provide rough or gross motion and the knob 1240 may be rotated to provide fine motion. In some embodiments, the handle 1230 is rotatable relative to the needle body 1202. Gearing (e.g., a worm gear, bevel gears, rack and pinion, etc.) can be used to translate the rotation of the handle 1230 into longitudinal movement of the stylet 1212. The handle may comprise detents, indicia, sensors, etc. to provide a user with information about the extent of extension of the stylet 1212, such as the indicia 1236.

FIG. 12F is partial top view of the tissue treatment device 1200 of FIG. 12E. The device 1200 may include a handle 1230. The handle 1230 may share features with the handle 630, 930, 1030 (e.g., electronic circuitry, gearing, etc.). The handle 1230 comprises a knob 1232 configured to slide in a channel 1234. Distally advancing the knob 1232 distally advances the stylet 1212. The handle 1230 comprises indicia 1236 to provide a user with information about the extent of extension of the stylet 1212 and/or the distance between the first electrode (e.g., formed from a second tube of the stylet 1212) and the second electrode (e.g., formed from the tip 1206). For example, the indicia 1236 aligned with the knob indicia 1237 in FIG. 12C can correspond to a first distance between the first electrode and the second electrode.

FIG. 12G is another partial side view the tissue treatment device 1200 of FIG. 12E. The treatment device 1200 may include a handle 1230. The handle 1230 may include a knob 1232 configured to slide in the channel 1234. The handle may also include a rotatable knob 1240. The handle 1230 may include a rotatable knob 1240. Gearing (e.g., a worm gear, bevel gears, rack and pinion, etc.) can be used to translate the rotation of the knob 1240 into longitudinal movement of the stylet 1212.

FIG. 12H is partial bottom view of the tissue treatment device of FIG. 12E. The treatment device 1200 may include a handle 1230. The handle 1230 may include a rotatable knob 1240 configured to control a longitudinal movement of the stylet 1212.

FIG. 12I is a side and detail view of a 5 mm deployment of the tissue treatment device 1200 of FIG. 12E. The treatment device 1200 comprises a needle body 1202. The distal end of the needle body 1202 comprises a tissue-penetrating tip 1204. In some embodiments, the tip 1204 may comprises a first electrode. In some embodiments, the tip 1204 and/or the needle body 1202 is free of an electrode. The treatment device 1200 may comprise a stylet 1212 that extends from an aperture of the needle body 1202, as discussed above in conjunction with FIG. 12A. The distal end of the stylet 1212 comprises a tissue-penetrating tip 1206. The tip 1206 comprises a second electrode. In some embodiments, the second electrode can be separate from the tip 1206, for example as described with respect to the device 600. In some embodiments, the stylet 1212 may comprise one or more telescoping tubes.

The device 1200 may include a handle 1230. The handle 1230 may share features with the handle 630, 930, 1030 (e.g., electronic circuitry, gearing, etc.). The handle 1230 comprises a knob 1232 configured to slide in a channel 1234. Distally advancing the knob 1232 distally advances the stylet 1212. In some embodiments, the handle 1230 comprises indicia to provide a user with information about the extent of extension of the stylet 1212 and/or the distance between the first electrode (e.g., formed at the tissue penetrating tip 1204 of the needle body 1202) and the second electrode (e.g., formed from the tip 1206). For example, the knob 1232 may be advanced distally within the channel 1234, extending the stylet such that there is a first distance between the first electrode and the second electrode. As shown in FIG. 12I, the stylet 1212 may be extended such that the first distance is equal to 5 mm. In some embodiments, the first distance may be defined as a distance between the stylet tip 1206 and the needle body 1202, i.e. a stylet length. For example, the distance between the tip 1206 and the needle body 1202 may be 5 mm. In some embodiments, the first distance may be defined as a distance that the stylet is axially extended at a proximal end with respect to the needle body 1202.

FIG. 12J is a side and detail view of a 20 mm deployment of the tissue treatment device 1200 of FIG. 12E. The device 1200 may include a handle 1230. The handle 1230 may share features with the handle 630, 930, 1030 (e.g., electronic circuitry, gearing, etc.). The handle 1230 comprises a knob 1232 configured to slide in a channel 1234. Distally advancing the knob 1232 distally advances the stylet 1212. In some embodiments, the handle 1230 comprises indicia to provide a user with information about the extent of extension of the stylet 1212 and/or the distance between the first electrode (e.g., formed at the tissue penetrating tip 1204 of the needle body 1202) and the second electrode (e.g., formed from the tip 1206). For example, the knob 1232 may be advanced distally within the channel 1234, extending the stylet such that there is a first distance between the first electrode and the second electrode. As shown in FIG. 12J, the stylet 1212 may be extended such that the first distance is equal to 20 mm. In some embodiments, the first distance may be defined as a distance between the stylet tip 1206 and the needle body 1202. For example, the distance between the tip 1206 and the needle body 1202 may be 20 mm.

FIG. 12K is a side and detail view of a 22 mm deployment of the tissue treatment device of FIG. 12E. The device 1200 may include a handle 1230. The handle 1230 may share features with the handle 630, 930, 1030 (e.g., electronic circuitry, gearing, etc.). The handle 1230 comprises a knob 1232 configured to slide in a channel 1234. Distally advancing the knob 1232 distally advances the stylet 1212. In some embodiments, the handle 1230 comprises indicia to provide a user with information about the extent of extension of the stylet 1212 and/or the distance between the first electrode (e.g., formed at the tissue penetrating tip 1204 of the needle body 1202) and the second electrode (e.g., formed from the tip 1206). For example, the knob 1232 may be advanced distally within the channel 1234, extending the stylet such that there is a first distance between the first electrode and the second electrode. As shown in FIG. 12K, the stylet 1212 may be extended such that the first distance is equal to 22 mm. In some embodiments, the first distance may be defined as a distance between the stylet tip 1206 and the needle body 1202. For example, the distance between the tip 1206 and the needle body 1202 may be 22 mm.

FIG. 12L is an isometric view of the tissue treatment device of FIG. 12E. The treatment device 1200 comprises a needle body 1202. The distal end of the needle body 1202 comprises a tissue-penetrating tip 1204. In some embodiments, the tip 1204 may comprises a first electrode. In some embodiments, the tip 1204 and/or the needle body 1202 is free of an electrode. The treatment device 1200 may comprise a stylet 1212 that extends from an aperture of the needle body 1202, as discussed above in conjunction with FIG. 12A. The distal end of the stylet 1212 comprises a tissue-penetrating tip 1206. The tip 1206 comprises a second electrode. In some embodiments, the second electrode can be separate from the tip 1206, for example as described with respect to the device 600. In some embodiments, the stylet 1212 may comprise one or more telescoping tubes.

The device 1200 may include a handle 1230. The handle 1230 may share features with the handle 630, 930, 1030 (e.g., electronic circuitry, gearing, etc.). The handle 1230 comprises a knob 1232 configured to slide in a channel 1234. Distally advancing the knob 1232 distally advances the stylet 1212. The handle 1230 comprises indicia 1236 to provide a user with information about the extent of extension of the stylet 1212 and/or the distance between the first electrode (e.g., formed from the tip 1205) and the second electrode (e.g., formed from the tip 1206). For example, the indicia 1236 aligned with the knob indicia 1237 in FIG. 12C can correspond to a first distance between the first electrode and the second electrode. In some embodiments, the first distance may be between about 1 mm and 25 mm (e.g., 1 mm, 5 mm, 8 mm, 10 mm, 13 mm, 15 mm, 18 mm, 20 mm, 22 mm, 25 mm, and other values and ranges therein).

FIG. 12M is a schematic view of cleaning a retracted tissue treatment device 1200 of FIG. 12E. The treatment device 1200 comprises a needle body 1202 that includes a tissue penetrating tip 1204. The needle body 1202 may house a stylet as discussed herein, which may not be extended during cleaning. The treatment device 1200 comprises a handle 1230. The handle 1230 may comprise an aperture 1242 configured to receive an insertion member 1244. The insertion member 1244 may be configured to mate with the geometry of the aperture 1242. In some embodiments, the insertion member 1244 may be a conical member that fits within the aperture 1242 and may be sealed within the aperture 1242 with friction. The insertion member 1244 may be threaded, and the aperture 1242 may correspondingly include threads to receive the insertion member 1242. In some embodiments, the insertion member 1244 may be a syringe that includes a needle, a conical member, and/or a threaded connection at the distal end of the syringe. The insertion member 1244 may deliver a fluid to the handle 1230 of the treatment device via the aperture 1242. The fluid may be configured to clean the needle body 1202. The insertion member 1244 may be further configured to withdraw the fluid from the handle 1230.

The handle 1230 may comprise a needle body channel 1246 configured to receive the needle body 1202. In some embodiments, the aperture 1242 may be in fluid communication with the needle body channel 1246 such that the fluid is delivered to the needle body 1202 and/or the stylet. For example, the insertion member 1244 may deliver a cleaning fluid to the needle body 1202 and/or the stylet via the aperture 1242. Within the needle body channel 1246, the cleaning fluid may clean and/or sterilize the needle body 1202 and/or the stylet. The insertion member 1244 may remove the fluid from the needle body channel 1246 once the needle body 1202 is cleaned and/or sterilized. In some embodiments, the fluid may evaporate within the needle body channel 1246 and may not be withdrawn by the insertion member 1244. The needle body 1202 may be moved within the needle body channel 1246 to facilitate a cleaning procedure and expose the needle body 1202 to the fluid. In some embodiments, the needle body channel 1246 may be in fluid communication with the distal end of the needle body 1202 such that the fluid inserted from the insertion member 1244 may be delivered to a treatment site. For example, the distal end of the needle body 1202 may include a second aperture in fluid connection with the aperture 1242 and the needle body channel 1246.

FIG. 12N is a schematic view of cleaning the retracted tissue treatment device 1200 of FIG. 12E. The treatment device 1200 comprises a handle 1230. The handle 1230 may comprise an aperture 1242 configured to receive an insertion member 1244. As shown in FIG. 12N, the aperture 1242 may be disposed on a lateral side of the handle 1230. The aperture 1242 may be disposed below a center line of the lateral side of the handle 1230. For example, the aperture 1242 may be disposed on a lateral side of the handle 1230, 45 degrees below a horizontal midline. The insertion member 1244 may be configured to mate with the geometry of the aperture 1242. As shown in FIG. 12N, the insertion member 1244 may be a syringe that includes a conical member configured to mate with an aperture 1242 of the handle 1230.

FIG. 12O is a schematic view of cleaning a retracted tissue treatment device 1200 of FIG. 12E. The treatment device 1200 comprises a needle body 1202 that includes a tissue penetrating tip 1204. In some embodiments, the tip 1204 may comprises a first electrode. In some embodiments, the tip 1204 and/or the needle body 1202 is free of an electrode. The treatment device 1200 may comprise a stylet 1212 that extends from an aperture of the needle body 1202, as discussed above in conjunction with FIG. 12A. The distal end of the stylet 1212 comprises a tissue-penetrating tip 1206. The tip 1206 comprises a second electrode.

The handle 1230 may comprise an aperture 1242 configured to receive an insertion member 1244. The insertion member 1244 may be configured to mate with the geometry of the aperture 1242. In some embodiments, the insertion member 1244 may be a syringe that includes a needle, a conical member, and/or a threaded connection at the distal end of the syringe. The insertion member 1244 may deliver a fluid to the handle 1230 of the treatment device. The fluid may be configured to clean the needle body 1202. The insertion member 1244 may be further configured to withdraw the fluid from the handle 1230. In some embodiments, the needle body 1202 and/or the stylet 1212 may be cleaned while the stylet is extended.

The handle 1230 may comprise a needle body channel 1246 configured to receive the needle body 1202. In some embodiments, the aperture 1242 may be in fluid communication with the needle body channel 1246 such that the fluid is delivered to the needle body 1202 and/or the stylet. For example, the insertion member 1244 may deliver a cleaning fluid to the needle body 1202 and/or the stylet via the aperture 1242. Within the needle body channel 1246, the cleaning fluid may clean and/or sterilize the needle body 1202 and/or the stylet. The insertion member 1244 may remove the fluid from the needle body channel 1246 once the needle body 1202 is cleaned and/or sterilized. In some embodiments, the fluid may evaporate within the needle body channel 1246 and may not be withdrawn by the insertion member 1244, FIG. 12P is a schematic view of cleaning the retracted tissue treatment device 1200 of FIG. 12E. The treatment device 1200 comprises a handle 1230. The handle 1230 may comprise an aperture 1242 configured to receive an insertion member 1244. As shown in FIG. 12P, the aperture 1242 may be disposed on a bottom side of the handle 1230. For example, the aperture 1242 may be at a midpoint of the bottom side of the handle 1230. In some embodiments, the aperture 1242 may be positioned based on the extension or non-extension of the stylet. In some embodiments, the handle may include two or more apertures such as a first aperture disposed at the position shown in FIGS. 12M-12N and a second aperture disposed at the position shown in FIGS. 12O-12P. In such embodiments, the insertion 1244 may be inserted into the first or second aperture based on the extension or non-extension of the stylet 1212. The insertion member 1244 may be configured to mate with the geometry of the aperture 1242. As shown in FIG. 12P, the insertion member 1244 may be a syringe that includes a conical member configured to mate with an aperture 1242 of the handle 1230. FIGS. 13A-13D are schematic depictions of example lesions. Different types of electrode devices produce different sizes and shapes of lesions, even when operated under the same or similar radiofrequency signals. In various embodiments, a treatment device may be an ablation device or probe (or other device suitable to deliver radiofrequency and/or other modalities for neuromodulation, such as heat, cryotherapy, microwave, ultrasound, chemical, etc.).

FIG. 13A shows an example lesion 1300 formed using a protruding electrode (PE) probe 1302 including an electrode 1304 deployed from a needle or cannula 1306. Monopolar energy was applied to both the electrode 1304 and the needle 1306 at 80° C. for 150 seconds. The energy produces a lesion 1300 having a thickness T₁ of 6.9 mm, a length L₁ of 11.7 mm, and a volume of 215mm 3 . The electrode 1304 is deployed to make the lesion 1300 larger, and the device does not emit bipolar energy between the probe 1302 and the electrode 1304.

FIG. 13B shows an example lesion 1310 formed using a cooled probe 1312. Monopolar energy was applied at 60° C. for 150 seconds. The energy produces a lesion 1310 having a thickness T₂ of 11.2 mm, a length L₂ of 10.7 mm, and a volume of 595mm³.

FIG. 13C shows an example lesion 1320 formed using a monopolar probe 1322. Monopolar energy was applied to the probe 1322 at 80° C. for 150 seconds. The energy produces a lesion 1320 having a thickness T₃ of 5.9 mm, a length L₃ of 10.8 mm, and a volume of 169mm³.

FIG. 13D shows an example lesion 1330 formed using a multi-tine (MT) probe 1332 including two electrodes 1334 deployed from a needle 1336. Monopolar energy was applied to the probe 1332 at 75° C. for 80 seconds. The energy produces a lesion 1330 having a thickness T₄ of 9 mm, a length L₄ of 10 mm, and a volume of 467mm³ . The electrodes 1334 are deployed to make the lesion 1330 larger, and the device does not emit bipolar energy between the probe 1332 and the electrodes 1334.

FIG. 13E is another schematic depiction of an example lesion 1350 using an ablation device 1352 having features described herein (e.g., the device 600, 800, 900, 1000, 1100, 1200). Bipolar energy was applied between a first electrode 1354 and a second electrode 1356 on a deployable stylet 1358. The energy produces a lesion 1350 having a thickness T₅ of 20 mm, a length L₅ of 5 mm, and a volume of 1,047mm³ . The lesion 1350 has a thickness T₅ that is significantly greater than (e.g., more than double) the thicknesses T₁, T₂, T₃, T₄. The thickness T₅ may be between about 1 mm and about 100 mm (e.g., about 1 mm, about 5 mm, about 10 mm, about 15 mm, about 20 mm, about 25 mm, about 30 mm, about 40 mm, about 50 mm, about 75 mm, about 100 mm, ranges between such values, and the like) depending on the subject being treated. For example, a mouse may have a very small hip joint capsule while an elephant may have a very large hip joint capsule. The lesion 1350 has a length L₅ that is significantly smaller than (e.g., less than half of) the lengths L₁, L₂, L₃, L₄. The length L₅ may be substantially constant over a range of thickness T₅. The lesion 1350 has a thickness T₅ to length L₅ (T₅:L₅) ratio of 20:5(4:1), which is significantly greater than (e.g., more the four times) the ratios T₁:L₁ (6.9/11.7(0.57:1), T₂:L₂ (11.2/10.7(1.05:1)), T₃:L₃ (5.9/10.8(0.55:1)), T₄:L₄ (9/10(0.9:1)). In some embodiments, a lesion produced by the devices described herein have a thickness to length ratio greater than about 1.25:1 and/or between about 1.25:1 and about 10:1 (e.g., about 1.25:1, about 1.5:1, about 2:1, about 2.5:1, about 3:1, about 3.5:1, about 4:1, about 4.5:1, about 5:1, about 6:1, about 8:1, about 10:1, ranges between such values, and the like). Greater thicknesses and/or ratios can be achieved, for example, by further extending an electrode on a stylet further from an electrode on a needle from which the stylet extends and/or further from an electrode on the stylet proximate to a needle from which the stylet extends. The lesion 1350 can have a thin long shape that is better suited for denervation and/or is more adjustable for the specific application than the shapes of the lesions 1300, 1310, 1320, 1330.

FIG. 13F is another schematic depiction of an example lesion 1360 using an ablation device 1352 having features described herein (e.g., the device 600, 800, 900, 1000, 1100, 1200). Bipolar energy was applied between a first electrode 1354 and a second electrode 1356 on a deployable stylet 1358. The energy produces a lesion 1350 having a thickness T₆, a length L₆ and a volume. For example, T₆ may be 20 mm, and L₆ may be 5 mm, resulting in a volume of 1,047mm³ . The lesion 1350 has a thickness T₆ that is significantly greater than (e.g., more than double) the thicknesses T₁, T₂, T₃, T₄. The thickness T₆ may be between about 1 mm and about 100 mm (e.g., about 1 mm, about 5 mm, about 10 mm, about 15 mm, about 20 mm, about 25 mm, about 30 mm, about 40 mm, about 50 mm, about 75 mm, about 100 mm, ranges between such values, and the like) depending on the subject being treated. For example, a mouse may have a very small hip joint capsule while an elephant may have a very large hip joint capsule. The lesion 1350 has a length L₆ that is significantly smaller than (e.g., less than half of) the lengths L₁, L₂, L₃, L₄. The length L₆ may be substantially constant over a range of thickness T₅. The lesion 1350 has a thickness T₆ to length L₆ (T₆:L₆) ratio of 20:5(4 :1), which is significantly greater than (e.g., more the four times) the ratios T₁:L₁ (6.9/11.7 (0.57:1), T₂:L₂ (11.2/10.7 (1.05:1), T₃:L₃ (5.9/10.8 (0.55:1)), T₄:L₄ (9/10 (0.9:1). In some embodiments, a lesion produced by the devices described herein have a thickness to length ratio greater than about 1.25:1 and/or between about 1.25:1 and about 10:1 (e.g., about 1.25:1, about 1.5:1, about 2:1, about 2.5:1, about 3:1, about 3.5:1, about 4:1, about 4.5:1, about 5:1, about 6:1, about 8:1, about 10:1, ranges between such values, and the like). Greater thicknesses and/or ratios can be achieved, for example, by further extending an electrode on a stylet further from an electrode on a needle from which the stylet extends and/or further from an electrode on the stylet proximate to a needle from which the stylet extends. The lesion 1360 can have a thin long shape that is better suited for denervation and/or is more adjustable for the specific application than the shapes of the lesions 1300, 1310, 1320, 1330.

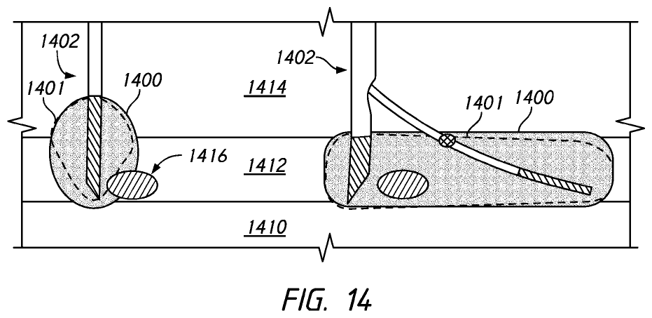

FIG. 14 is a schematic front (left) and side (right) depiction of example lesion 1400 using an ablation device 1402 having features described herein (e.g., the device 600, 800, 900, 1000, 1100, 1200). The area bounded by the solid line is the actual lesion 1400 and the area bounded by the dashed line is the expected lesion 1401. The expected lesion 1400 and the actual lesion 1401 are very close to each other. The device 1402 is designed specifically for the anatomy and tissue composition. For example, FIG. 14 also shows layers including bone 1410 (0.03 Siemens/meter (S/m) conductivity), adipose tissue 1412 (0.1 S/m), and muscle 1414 (0.54 S/m), and schematically shows the connective tissue layers 1416 protecting the nerve including the epineurium (0.08 S/m), endoneurium (0.08 S/m), and perineurium (0.002 S/m). The device 1402 focuses the electrical current in the target area and across the nerve, reduces (e.g., minimizes) current loss and/or damage to muscle 1414, and/or creates a more reproducible ablation zone 1401.

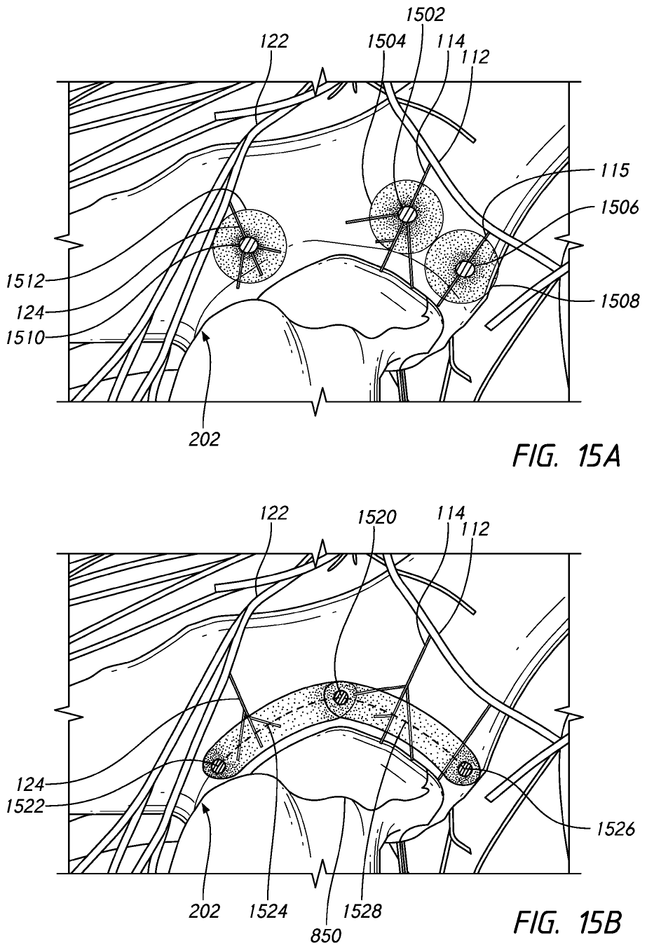

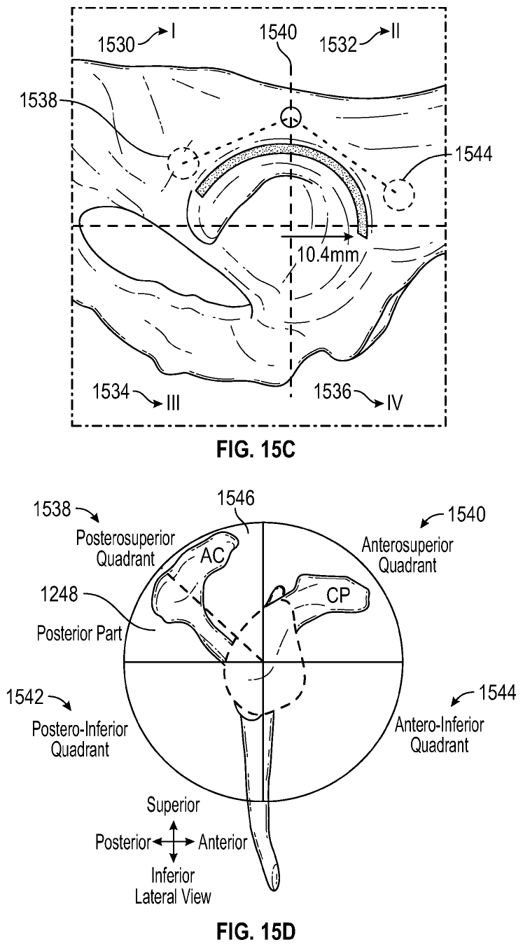

FIG. 15A is an example method of treating hip joint pain. For example, the method illustrated in FIG. 15A may be performed using one or more of the probes 1302, 1312, 1322, 1332 to form one or more of the lesions 1300, 1310, 1320, 1330. The method comprises modulating (e.g., denervating, ablating, etc.) an articular branch 124 from the sciatic nerve 122 and an articular branch 114 from the cranial gluteal nerve 112. The method optionally comprises modulating an articular branch 104 from femoral nerve 102 (e.g., FIGS. 1A, 1B, 8A). The method optionally comprises modulating an articular branch 134 from the obturator nerve 132 (e.g., FIGS. 1B, 5, 8B).

A user can insert an embodiment of a treatment device or probe at a first point 1502. The first point 1502 may attempt to target the articular branch 114. Facets of the hip joint capsule 202 may be used to help determine a position of the first point 1502, although it may be difficult to use landmarks for positioning. Because the T:L ratio of the lesion will be less than 1.25, the orientation of the device is not important. Imaging (e.g., fluoroscopy, ultrasound, etc.) may be used to verify the position of the treatment device prior to modulation. A lesion 1504 may then be created by applying energy to the treatment device. The lesion 1504 has a T:L ratio less than 1.25 and extends into the muscle. If the first point 1502 is not well targeted, the articular branch 114 may be entirely missed, as the low T:L ratio generally cannot account for nerve location variability. The user can insert the treatment device at a second point 1506 to attempt to modulate a second articular branch 115. A lesion 1508 may then be created by applying energy to the treatment device. The lesion 1508 has a T:L ratio less than 1.25 and extends into the muscle. If the second point 1504 is not well targeted, the articular branch 115 may be entirely missed. The user can insert the treatment device at a third point 1510 to attempt to modulate the articular branch 124. A lesion 1512 may then be created by applying energy to the treatment device. The lesion 1512 has a T:L ratio less than 1.25 and extends into the muscle. If the third point 1510 is not well targeted, the articular branch 124 may be entirely missed. The user therefore may have performed at least three separate insertions and at least three ablations and missed the articular branches 114, 115, 124 altogether, but affected muscle tissue around at least three points of the hip joint capsule 202. Ablating muscle tissue can cause additional pain. The lesions 1504, 1508, 1512 may also or alternatively ablate critical structures.

FIG. 15B is another example method of treating hip joint pain. The method of FIG. 15B may share features with the method of FIGS. 8A and 8B. The method comprises modulating (e.g., denervating, ablating, etc.) an articular branch 124 from the sciatic nerve 122 and an articular branch 114 from the cranial gluteal nerve 112. The method optionally comprises modulating an articular branch 104 from femoral nerve 102 (e.g., FIGS. 1A, 1B, 8A). The method optionally comprises modulating an articular branch 134 from the obturator nerve 132 (e.g., FIGS. 1B, 5, 8B).

Using a dorsal and caudal approach, a user can insert a treatment device as described herein (e.g., the device 600, 800, 900, 1000, 1100, 1200) at a first point 1520. The first point 1520 may be an apex of a hip joint capsule. The user can advance the treatment device until the distal end contacts bone. Other approaches are also possible. Facets of the hip joint capsule 202 may be used to help determine a position of the first point 1520. Imaging can help to orient the treatment device so that the stylet is deployed until the second point 1522. The acetabulum/acetabular rim (socket) and femoral head 850, for example, may be used to help determine a position of the second point 1522. The acetabulum is a concave/curved surface. The devices described herein may be uniquely designed to allow electrode positioning on bone around a concave/curved structure. The deployed stylet may extend distal to the tip of the needle body, for example by curving downward along a bone. Imaging (e.g., fluoroscopy, ultrasound, etc.) may be used to verify the position of the treatment device prior to modulation. Terms such as “first,” second, “third,” etc. provide nomenclature that can help distinguish between different points or other multiple items discussed herein, and do not necessarily describe an order, a preference, a hierarchy, etc. For example, in some embodiments, the treatment device may be inserted at the second point 1522 and extended to the first point 1520. For example, the second point 1522 may be easier to identify than the first point 1520 when percutaneously positioning the treatment device. The acetabulum is bounded dorsally, cranially, and caudally by the acetabular rim. The targeted modulations may occur just above this rim when targeting the articular branch 124 of the sciatic nerve 122 and/or the articular branch 114 of the cranial gluteal nerve 112. Using borders (e.g., dorsal, cranial, caudal) of the hip joint or acetabulum can help to identify the rim, which may be termed the dorsal rim and/or the ventral rim). Although certain examples of insertion and extension points are provided herein, other insertion and extension points are also possible, for example others that would create a path that would include an articular branch nerve of interest. Bipolar radiofrequency energy can be applied between the first electrode and the second electrode. The energy will extend along the path 1524, forming a substantially linear or curved/arcuate ablation zone (e.g., having a T:L ratio greater than about 1.25, as described with respect to FIGS. 13E and 14; measuring the curved thickness of the outside of the ablation zone in the case of curved/arcuate). The curvature of the ablation zone may follow the curvature of the bone, and bone is reflective of RF energy. The articular branch 124 from the sciatic nerve 122 lies in the path 1524 such that the RF energy can modulate the articular branch 124. The stylet can be retracted back into the needle body. After modulating the articular branch nerve 124, the pain is reduced. Pain reduction can be measured, for example, by walking evaluation, a biped station, rotation with external abduction, subluxation and iliopsoas, combinations thereof, a distraction index, etc. The test(s) can be performed before a procedure to establish a preoperative baseline, and then at one or more intervals after the procedure (e.g., one day, two days, one week, two weeks, one month, three months, six months, etc.). Imaging can also or alternatively be used to evaluate the tissue for signs of recovery.

In some embodiments, the user can rotate the treatment device (e.g., between about 100° and about 160°) to orient the treatment device so that the stylet is deployed until the third point 1526. Facets of the hip joint capsule 202 may be used to help determine a position of the third point 1526. Imaging (e.g., fluoroscopy, ultrasound, etc.) may be used to verify the position of the treatment device prior to modulation. Bipolar radiofrequency energy can be applied between the first electrode and the second electrode. The energy will extend along the path 1528, forming a substantially linear or curved/arcuate ablation zone (e.g., having a T:L ratio greater than about 1.25, as described with respect to FIGS. 13E and 14; measuring the curved thickness of the outside of the ablation zone in the case of curved/arcuate). The curvature of the ablation zone may follow the curvature of the bone, and bone is reflective of RF energy The articular branch 114 from the cranial gluteal nerve 112 lies in the path 1528 such that the RF energy can modulate the articular branch 114. After modulating the articular branch nerve 114, the pain is reduced. The stylet can be retracted back into the needle body. Rotating the treatment device between energy application can reduce the number of puncture sites (e.g., only a single puncture site to denervate the entire hip joint capsule 202), which can reduce soft tissue trauma and a number of possible infection sites. A smaller amount of rotation is also possible, for example to make sure that the first tissue was treated enough to capture the articular branch nerve. The paths 1524, 1528 extending along the hip joint capsule 202 can account for any nerve location variation that could occur. If the capsule 202 as illustrated in FIG. 15B is considered a clock face, the lesions may be created between the 12:00 position and the 3:00 position and between the 12:00 position and the 9:00 position, for example. The method can ablate two segments or paths to ablate two quadrants of a capsule from a single percutaneous insertion position. The lesions created along the paths 1524, 1528 can direct ablation away from critical structures and muscle. The method can simplify positioning using visible anatomical landmarks. The ablation zones have an adjustable length to account for various sizes of capsules 202 from subject to subject and across various species of subjects.

FIG. 15C depicts another exemplary embodiment demonstrating a quadrant approach to treating the hip of a subject. The hip of the subject may be broken into quadrants relative to one or more anatomical planes, such as the medial plane, the transverse plane, and/or the dorsal plane. One of the anatomical planes may be used as a dividing plane, and the remaining two anatomical planes may define the quadrants. For example, the medial plane may divide a joint of the subject, such as the hip, parallel to the medial plane. The joint may be divided into four quadrants based on a central point of the joint and the remaining two anatomical planes. Returning to the example, the hip may be divided into a posterior-superior quadrant, an anterosuperior quadrant, a postero-inferior quadrant, and an antero-inferior quadrant.

Similarly, the hip shown in FIG. 15C may be divided into four quadrants using a plane. The four quadrants may be a first quadrant 1530, a second quadrant 1532, a third quadrant 1534, and a fourth quadrant 1536. The user may insert a treatment device (e.g., the device 600, 800, 900, 1000, 1100, 1200) at a first point 1538 in the first quadrant 1530. The user may insert the treatment device until the treatment device reaches bone. Facets of the hip joint capsule may be used to help determine a position of the first point 1538. A stylet of the treatment device may be extended until it reaches a second point 1540. The second point 1540 may correspond with an intersection of the first quadrant 1530 and the second quadrant 1532. In some embodiments, the stylet extended from the second point 1540 to the third point 1544, which may be disposed in the second quadrant 1532. As discussed herein, the stylet may be shaped or adapted such that the stylet may be disposed between the first point 1538, the second point 1540, and the third point 1544.

Imaging can help to orient the treatment device so that the stylet is deployed between the first point, 1538, the second point 1540, and the third point 1544. The acetabulum/acetabular rim (socket) and femoral head, for example, may be used to help determine a position of the second point 1540 and/or the third point 1544. The acetabulum is a concave/curved surface. The devices described herein may be uniquely designed to allow electrode positioning on bone around a concave/curved structure. The deployed stylet may extend distal to the tip of the needle body, for example by curving downward along a bone. Imaging (e.g., fluoroscopy, ultrasound, etc.) may be used to verify the position of the treatment device prior to modulation.

The treatment device may be positioned in the first quadrant and second quadrant such that the stylet is above the acetabulum, allowing the device to treat the articular branch of the sciatic nerve and/or the articular branch of the cranial gluteal nerve, as discussed above in conjunction with FIGS. 15A and 15B, at the same time. In some embodiments, the treatment device may treat tissue in one or more of the quadrants at the same time, such as tissue in the third quadrant 1534 and the fourth quadrant 1536.

FIG. 15D depicts an exemplary embodiment of treatment of a joint in one, two, three, or four quadrants. Similar to the example given above, the joint in FIG. 15D may be split by the medial plane and divided into four quadrants by the transverse plane and the dorsal plane. The joint may be divided into a first quadrant 1538, a second quadrant 1540, a third quadrant 1542, and a fourth quadrant 1544. The first quadrant 1538 may be the posterosuperior quadrant. The second quadrant 1540 may be the anterosuperior quadrant. The third quadrant 1542 may be the postero-inferior quadrant, and the fourth quadrant 1544 may be the antero-inferior quadrant. In some embodiments each quadrant may be further divided into a first portion and a second portion. The first and second portion may also be defined by their proximity to an anatomical plane. For example, the first quadrant 1538 may be divided into a posterior portion 1546 and a superior portion 1548. In some embodiments, the treatment device may be used to target specific quadrant(s) or portion(s) of a quadrant. In various embodiments, any joint (e.g., hip, shoulder, elbow, knee, wrist, ankle, stifle, carpal joint, hock, etc.) may be targeted with a quadrant approach.

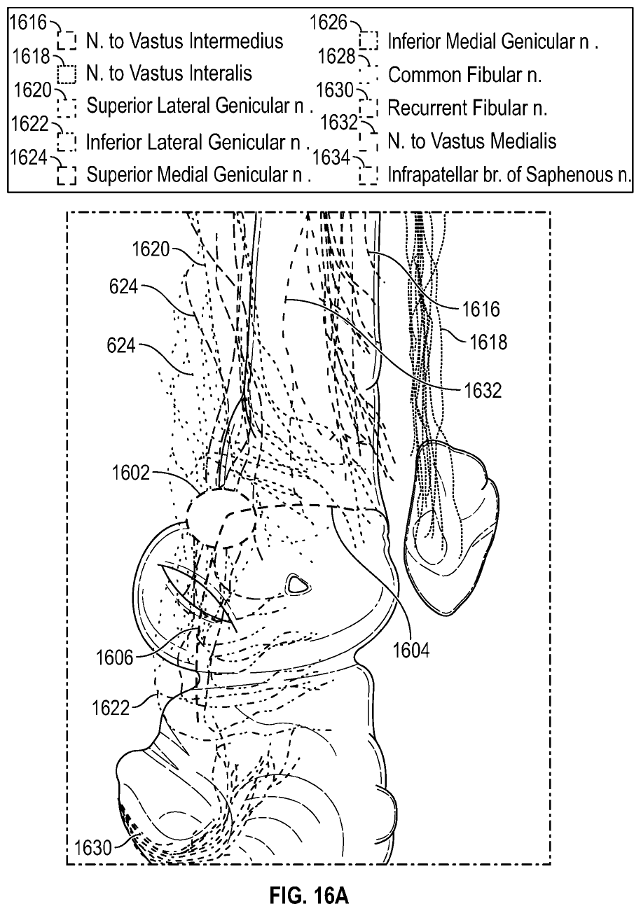

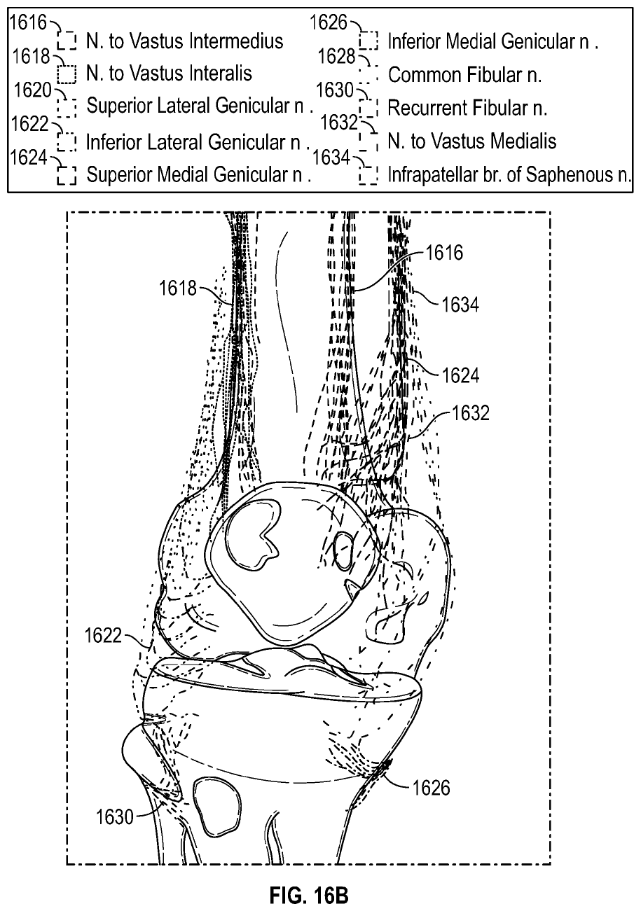

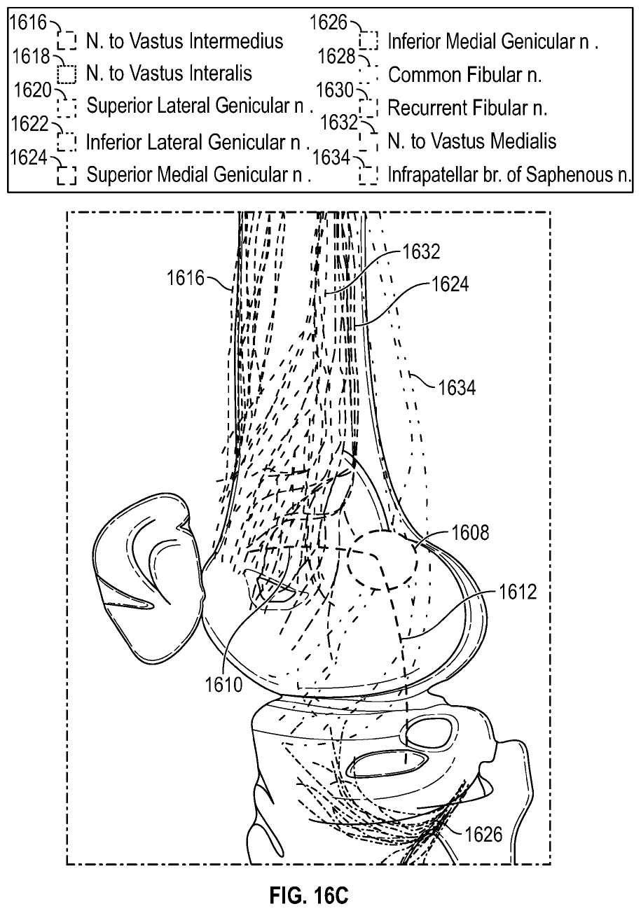

FIGS. 16A, 16B, and 16C illustrate an exemplary approach for treating knee pain in a subject using a lateral insertion (16A) and/or a medial insertion (16C) of the treatment device. At the knee, articular branches of nerves run along, or are immediately adjacent, to a cortical surface of bone as the branches approach the joint (as shown by the color-coded lines). In some embodiments, a treatment device (e.g., the device 600, 800, 900, 1000, 1100, 1200) may be inserted along a medial or lateral side of a knee 1600. For example, as shown in FIG. 16A, the treatment device may be inserted at a first point 1602 until a needle body of the treatment device contacts bone. For example, the first point 1602 may be a point on the lateral side of the subject's knee. The first point 1602 may be disposed on the posterior side of the knee superior to the joint capsule. A stylet of the treatment device may be extended to a first ablation region 1604. In some embodiments, the first ablation region 1604 may correspond with a lateral articular surface. The treatment device may include a first electrode disposed along a needle body and a second electrode disposed within the stylet. A bipolar radiofrequency energy may be applied to the first electrode and the second electrode to ablate tissue disposed within the first ablation region 1604. Similar to the embodiments described above in conjunction with a subject's hip, a user may reposition or rotate the stylet to a second ablation region 1606 and apply a bipolar radiofrequency to the tissue. The second ablation region 1606 may be disposed across a lateral side of the joint capsule. In some embodiments, the stylet may be extended along the second ablation region 1606 before the first ablation region 1604. As discussed above, in some embodiments, the stylet may be configured to ablate the first ablation region 1604 and the second ablation region 1606 at the same time.

FIG. 16B depicts an anterior view of the subject's knee, and FIG. 16C depicts a medial view of the subject's knee along with a second point 1608, a third ablation region 1610, and a fourth ablation region 1612. Similar to the process described above in conjunction with FIG. 16A. A treatment device may be inserted at the second point 1608 to treat the medial side of the subject's knee in a manner that is substantially similar to the lateral side as described in conjunction with FIG. 16A. The second point 1608 may be disposed on the medial side of the subject's knee. The second point 1610 may be posterior to the joint and disposed superior to the joint capsule. The third ablation region 1610 may correspond with a medial articular surface. The fourth ablation region 1612 may correspond with a medial side of the joint capsule. The third ablation region 1610 and the fourth ablation region 1612 may be treated in the same manner as the first ablation region 1604 and the second 1606 while accounting for the anatomical differences between the lateral and medial sides of the subject's knee.

Various nerves 1614 around a knee are shown in FIGS. 16A, 16B, and 16C. Nerve 1616 corresponds with nerves to the vastus intermedius. Nerve 1618 corresponds with nerves to the vastus lateralis. Nerve 1620 corresponds with the superior lateral genicular nerve. Nerve 1622 corresponds with the inferior lateral genicular nerve. Nerve 1624 corresponds with the superior medial genicular nerve. Nerve 1626 corresponds with the inferior medial genicular nerve. Nerve 1628 corresponds with the common fibular nerve. Nerve 1630 corresponds with the recurrent fibular nerve. Nerve 1632 corresponds with the nerves tot the vastus medialis. Nerve 1634 corresponds with the infrapatellar branch of the saphenous nerve. In some embodiments, the first ablation region 1604 may target the common fibular nerve, the superior lateral genicular nerve, the nerves to the vastus intermedius and/or the nerves to the vastus lateralis. The second ablation region 1606 may target the common fibular nerve, the inferior lateral genicular nerve, and/or the recurrent fibular nerve. In some embodiments, the third ablation region 1610 may target the nerves to the vastus intermedius, the nerves to the vastus medialis, the superior medial genicular nerve, and/or the infrapatellar branch of the saphenous nerve. The fourth ablation region 1612 may target the inferior medial genicular nerve and/or the infrapatellar branch of the saphenous nerve.

Advantageously, ablating in an area close to joint capsule attachment or across the capsule limits targets to the nerves of the joint capsule. In turn, vascular damage can be reduced because vessel branches to areas like nutrient foramen and metaphysis points have already branched and most of the blood supply to the capsule may not be present. Moreover, the identified ablation regions avoid ligaments, tendons, and their associated attachment points near the joint capsule. Furthermore, ablating where nerve is close to bone greatly increases likelihood of capturing nerve, provides easy anatomical reference points, and allows use of lower profile ablation zone (proximal/distal direction) which protects tissues/structures nearby.

In some embodiments, knee pain may be treated by ablating a plurality of nociceptors. The plurality of nociceptors may be disposed within the joint capsule, outside the joint capsule, at a tendon or ligament attachment, at an articular surface, in a fibrous layer of the joint capsule, or a tissue associated with the knee. In some embodiments, sympathetic nervous tissue is not be ablated. The sympathetic nerves may be in close proximity to vessels and can be found in the synovium below fibrous layers. For example, the plurality of nociceptors may be disposed in a fibrous layer of the joint capsule where sympathetic nerves may not be present. The fibrous layer may be ablated, and the sympathetic nerves may be spared along with other tissues in the synovium. In some embodiments a treatment device (e.g., the device 600, 800, 900, 1000, 1100, 1200) may be used to ablate the plurality of nociceptors.

Advantageously, this approach results in very minimal disruption to other structures, especially ligaments, tendons, arteries, veins, and nerves. Selective targeting of nociceptive nerve endings while sparing the sympathetic nerves may ensure that the subject do not suffer complications from lack of pressure, stability, motor feedback, or vasoconstriction.

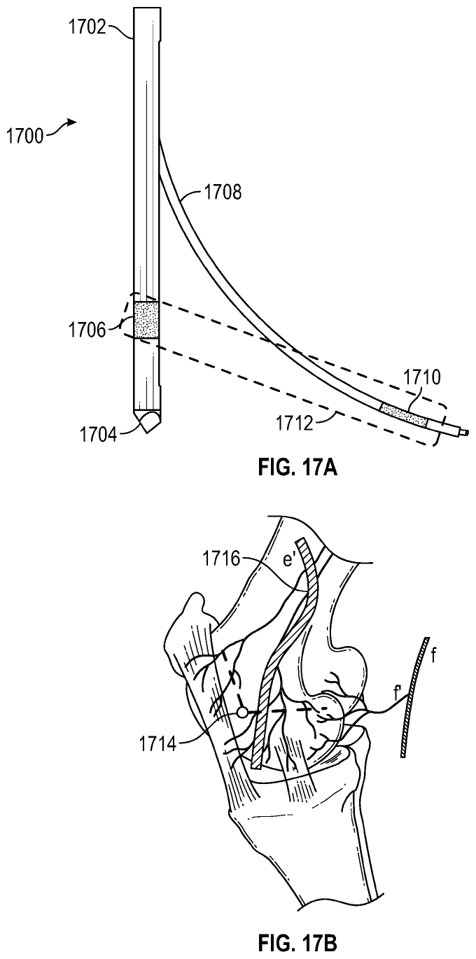

FIG. 17A depicts an exemplary treatment device 1700. The treatment device 1700 may be configured similarly to other embodiments described herein (e.g., the device 600, 800, 900, 1000, 1100, 1200 and any probe herein). The treatment device 1700 may comprise a needle body 1702 and a stylet 1708. The needle body 1704 may have a tissue-penetrating tip 1704 and a first electrode 1706. The first electrode 1706 may be disposed above the tissue-penetrating tip 1704 by a first distance. For example, the first distance between the tip 1704 and the first electrode 1706 may be 0.1 inches, 0.2 inches, 0.25 inches, 0.5 inches, 0.75 inches, 1.0 inch, 1.25 inches, 1.5 inches, 1.75 inches, 2.0 inches. Similarly, the stylet 1708 may comprise a second electrode 1710 that is disposed along the body of the stylet 1708 and offset from the tip of the stylus 1708 by a second distance. The second distance between the second electrode 1710 and the tip of the stylet 1708 may be 0.1 inches, 0.2 inches, 0.25 inches, 0.3 inches, 0.4 inches, 0.5 inches, 0.6 inches, 0.7 inches, 0.75 inches 0.8 inches, 0.9 inches, or 1.0 inch. An ablation area 1712 may lie between the first electrode 1706 and the second electrode 1710. The placement of the first electrode 1706 along the needle body 1708 and the placement the second electrode 1710 along the body of the stylet 1708 may define features of the ablation area 1712. Features of the ablation area 1712 may include a length, a width, and/or an angle between the needle body 1702 and the ablation area 1712. By placing the electrodes more proximal from the distal tips, the ablation area 1712 may be advantageously elevated off of a bone surface to avoid critical structures.

FIG. 17B illustrates how the treatment device 1700 may be used to target certain anatomical areas of a subject without damaging critical structures. For example, the treatment device 1700 may be used to ablate tissue along the dotted lines 1714. The elevation of the ablation area 1712 may allow for the tissue proximal to the dotted line 1714 to be ablated while the vessel 1716 underneath the ablation area 1712 is spared.

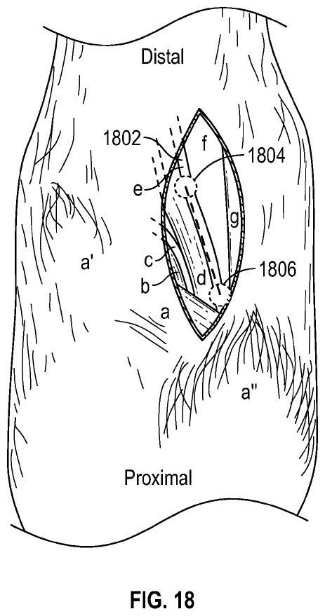

FIG. 18 demonstrates an exemplary embodiment where a treatment device (e.g., the device 600, 800, 900, 1000, 1100, 1200, 1700 and any probe herein) may be used to treat a nerve tissue 1802. A needle body of the treatment device may be inserted along a nerve 1802 at a first point 1804. The first point 1804 may be a proximal point along an extended length of the nerve 1802. The stylet of the treatment device may be extended to a second point 1806. The second point 1806 may be disposed along the nerve 1802 more distal than the first point 1804. A bipolar radiofrequency may be applied between the first point 1804 and the second point 1806 such that the nerve 1802 is ablated along its length. Advantageously, the treatment device allows for targeting of the nerve 1802 while collateral damage to other tissues may be minimized. Moreover, ablation of a nerve lengthwise may provide more sustained relief to a subject.

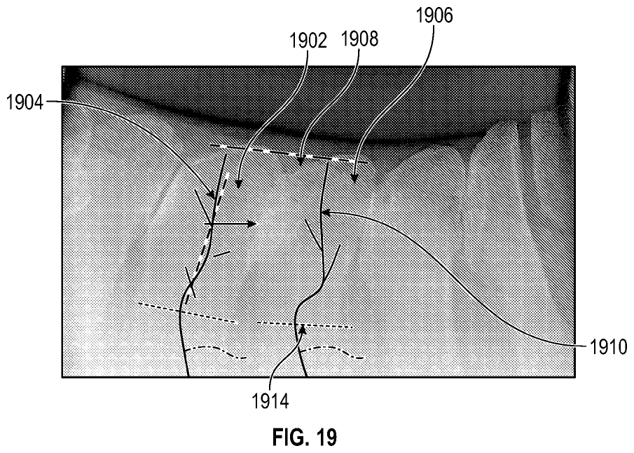

FIG. 19 depicts an exemplary embodiment 1900 where a treatment device (e.g., the device 600, 800, 900, 1000, 1100, 1200, 1700 and any probe herein) may be used to modulate the sensory nerves of a spinous process. A needle body may be inserted at a first spinous process 1902. In some embodiments, the stylet of the treatment device may be extended along the first spinous process and a nerve tissue 1904 (shown by the dotted line). A bipolar radiofrequency may be generated between a first electrode on the needle body and a second electrode on the stylet such that the nerve tissue 1904 is ablated. The nerve tissue 1904 may be one of: a dorsal ramus of the first vertebrae, a lateral dorsal ramus of the first vertebrae, and/or a medial dorsal ramus of the first vertebrae. In some embodiments, the medial branch of the dorsal ramus may be spared from ablation.

In some embodiments, the needle body may be inserted at the first spinous process 1902, and the stylet may be extended to a second spinous process 1906 such that the treatment device is in a first position 1908. In a similar manner, a bipolar radiofrequency may be generated between a first electrode on the needle body and a second electrode on the stylet. The bipolar radiofrequency may ablate the first nerve tissue 1904 of the first spinous process 1902 and second nerve tissue 1910 of the second spinous process 1906. In some embodiments, the ablation area, i.e. the area between the first electrode and the second electrode, may be deeper along the first spinous process 1902 and the second spinous process 1906. For example, the ablation area may be along the dotted lines 1914.

Advantageously, in several embodiments, modulation of nerve tissue along the spinous process may be a minimally invasive alternative to invasive surgical procedures. For example, the method described in FIG. 19 may be used treat spinous process impingement, or “kissing spine,” in horses as a minimally invasive alternative to a desmotomy or other invasive surgical procedures. Additionally, modulation of nerve tissue along the spinous process and vertebrae may provide pain relief to a subject with a shorter recovery time and greater efficacy.

A treatment device as described herein (e.g., the device 600, 800, 900, 1000, 1100, 1200, 1700 and any probe herein) may be used to modify tissue associated with various joints. For example, the treatment device may be used to modify articular branches of nerves associated with a hip, a knee, a vertebral joint, a stifle, an elbow, a shoulder, a metacarpophalangeal joint, a metatarsophalangeal joint, a hock, a coffin joint, a wrist, an ankle, etc. In some embodiments, the treatment device may be adapted to the anatomy of the joint. The treatment device may be inserted near the joint, and the stylet may be extended such that a target nerve associated with the joint is positioned between the first electrode and the second electrode. A bipolar radiofrequency may be applied between the first electrode and the second electrode, ablating the nerve tissue. The following figures are exemplary placements of the treatment device for various joints. Although specific tissues and locations are indicated, the examples are not intended to limit the functionality of the treatment device or the manner in which the treatment device may be used to treat a particular joint.

### Quadruped Stifle

The quadruped stifle roughly corresponds to a knee. The stifle comprises several articular nerves.

The medial articular nerve is usually in the first branch of the saphenous nerve, and is approximately ⅔ of the diameter of the saphenous nerve. Its course from the saphenous nerve to the knee joint is in the connective tissue interval between the anterior aspect of the adductor magnus and brevis muscle and the posterior surface of vastus medialis. It runs deep to the separation between the cranial and caudal portions of the satorius muscle in company with the descending genicular artery and vein. At about the level of the proximal attachment of the medial collateral ligament the medial articular nerve branches towards its various destinations. Branches go to the MCL, other branches are traced to the capsular tissue of the anterior, medial and posterior aspects of the joint, while some of these branches could be further traced to their apparent terminations within the capsule proper, the infrapatellar fat pad, or their apparent destinations in the attachments of the cruciate ligaments or meniscal horns. The medial articular nerve may be approached surgically. The groovelike depression immediately posterior to the firm belly of vastus medialis may be identified by firmly sliding one's finger posteriorly on the medial aspect of the thigh, beginning anteriorly. A 2-3 cm long incision may be made in the middle third of the thigh along the anterior edge of this depression to expose vastus medialis. The surface of the muscle may be followed posterior and deep until the descending geniculate artery and vein are identified, at which depth the medial articular nerve may easily be found. No muscle, large artery, vein or nerve is endangered using this approach

The posterior articular nerve is generally smaller than the corresponding medial articular nerve, and as a group the posterior articular nerves vary both with regard to the number of roots from which they are formed and their point of origin. The posterior articular nerves may be approached surgically as follows: with the animal lying prone, the depression marking the popliteal fossa is identified, and by palpation is traced cranially and laterally. This extension of the popliteal fossa represents the division between the biceps femoris laterally and the semimembranosus and semitendinosus medially. An incision is made from approximately mid-thigh to the lowermost extent of the popliteal fossa, and the sciatic nerve is exposed cranially by retracting the biceps femoris laterally and the semimembranosus and semitendinosus medially. The bifurcation of the tibial and common peroneal nerves is identified, and the posterior articular nerve is sought as it departs from the deep surface of the tibial nerve. As with the surgical exposure of the medial articular nerve, no major blood vessels or nerves need be disturbed.

The lateral articular nerve arises as one or several branches from the common peroneal nerve about 2 cm before it wraps around the neck of the fibula. It sometimes consists of one, two, three, four, or five branches. Each such branch travels superiorly to supply the superior tibiofibular joint, the lateral collateral ligament, or the lateral or posterolateral joint capsule. Occasional branches could be traced through the capsular tissue to the periphery of the lateral meniscus. The lateral articular nerve may be approached surgically, but often by damaging the most distal fibers of the biceps femoris muscle. Thus, the common peroneal nerve is identified by palpation as it wraps around the neck of the fibula, and an incision is made immediately over the nerve. The fibers of the biceps femoris muscle are then separated to expose the common peroneal nerve, and the articular nerves springing from it.

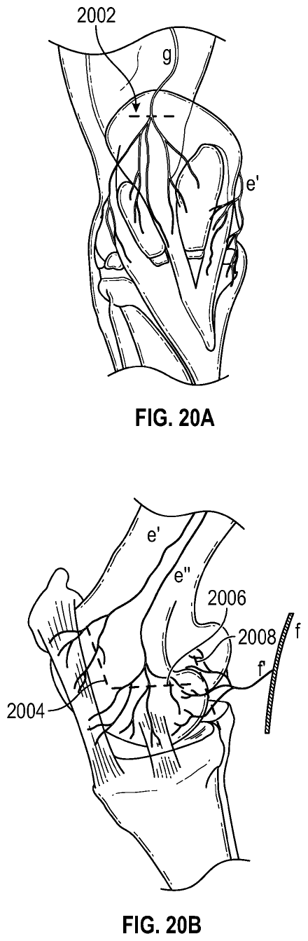

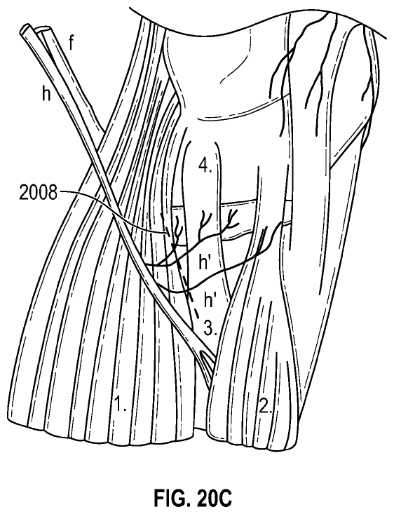

FIG. 20A illustrates an example of placement of a probe via an anterior view of an equine stifle. The treatment device (e.g., the device 600, 800, 900, 1000, 1100, 1200, 1700 and any probe herein) may be used to modify the femoral nerve. A user may modify the femoral nerve by disposing the first electrode and the second electrode of the treatment device along the dotted line 2002, which may be disposed proximal to the stifle, and generating a bipolar radiofrequency between the first and second electrode.

FIG. 20B illustrates an example of placement of a probe via a medial view of an equine stifle. The treatment device (e.g., the device 600, 800, 900, 1000, 1100, 1200, 1700 and any probe herein) may be used to modify nerves associated with the stifle. A user may modify the cranial branch of the medial articular nerve by disposing the first electrode and the second electrode of the treatment device along the dotted line 2004 and generating a bipolar radiofrequency between the first and second electrode. In some embodiments, a user may modify the caudal branch of the medial articular nerve by disposing the first electrode and the second electrode of the treatment device along the dotted line 2006 and generating a bipolar radiofrequency between the first and second electrode. A user may modify the articular branch of the tibial nerve by disposing the first electrode and the second electrode of the treatment device along the dotted line 2008 and generating a bipolar radiofrequency between the first and second electrode. In some embodiments, a user may ablate along one or more of the dotted lines 2004, 2006, and 2008. The treatment device may be adapted to treat two or more areas at once, such as a first area along the dotted line 2004 and a second area along the dotted line 2006.

FIG. 20C illustrates an example of placement of a probe via a lateral view of an equine stifle. The treatment device (e.g., the device 600, 800, 900, 1000, 1100, 1200, 1700 and any probe herein) may be used to modify nerves associated with the stifle. A user may modify one or more of the articular branches of the common peroneal nerve by disposing the first electrode and the second electrode of the treatment device along the dotted line 2010 and generating a bipolar radiofrequency between the first and second electrode.

In some embodiments, a user may curatively or palliatively treat the stifle by ablating the joint along one or more of the lines 2002, 2004, 2006, 2008, and 2010. For example, the stifle may be treated at the dotted lines 2002, 2004, 2006, 2008, and/or 2010 to treat osteoarthritis, pain, or other conditions.

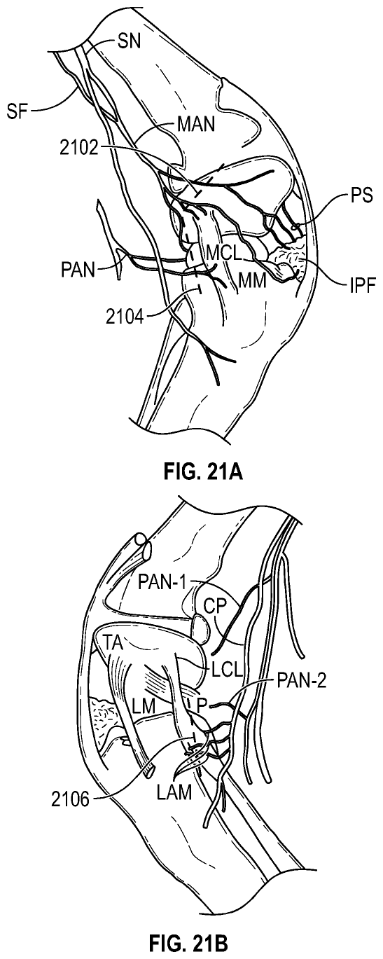

FIG. 21A illustrates an example of placement of a probe via a medial view of a canine knee. The treatment device (e.g., the device 600, 800, 900, 1000, 1100, 1200, 1700 and any probe herein) may be used to modify nerves associated with the knee. A user may modify one or more of the branches of the medial articular nerve and/or one or more branches of the posterior articular nerve by disposing the first electrode and the second electrode of the treatment device along the dotted line 2102 and the dotted line 2104, respectively. The user may ablate the branches by generating a bipolar radiofrequency between the first and second electrode. In some embodiments, the treatment device may be configured to ablate a first area along the dotted line 2102 and a second area along the dotted line 2104 in a single pulse.

In some embodiments, the medial articular nerve may easily be approached surgically. The groovelike depression immediately posterior to the firm belly of vastus medialis may be identified by firmly sliding one's finger posteriorly on the medial aspect of the thigh, beginning anteriorly. A 2-3 cm long incision may be made in the middle third of the thigh along the anterior edge of this depression to expose vastus medialis. The surface of the muscle may be followed posterior and deep until the descending geniculate artery and vein are identified, at which depth the medial articular nerve may easily be found. No major blood vessels or nerves may be disturbed by this approach.

The posterior articular nerve(s) may be approached surgically according to some embodiments. With the subject lying prone, the depression marking the popliteal fossa may be identified and may be traced, by palpation, cranially and laterally. This extension of the popliteal fossa represents the division between the biceps femoris laterally and the semimembranosus and semitendinosus medially. An incision may be made from approximately mid-thigh to the lowermost extent of the popliteal fossa, and the sciatic nerve may be exposed cranially by retracting the biceps femoris laterally and the semimembranosus and semitendinosus medially. The bifurcation of the tibial and common peroneal nerves may be identified, and the posterior articular nerve may be sought as it departs from the deep surface of the tibial nerve. As with the surgical exposure of the medial articular nerve, no major blood vessels or nerves need be disturbed.

FIG. 21B illustrates an example of placement of a probe via a lateral view of a canine knee. The treatment device (e.g., the device 600, 800, 900, 1000, 1100, 1200, 1700 and any probe herein) may be used to modify nerves associated with the knee. A user may modify one or more of the branches of the lateral articular nerve a by disposing the first electrode and the second electrode of the treatment device along the dotted line 2106 and generating a bipolar radiofrequency between the first and second electrode. In some embodiments, the lateral articular nerve may be approached surgically. In some embodiments the common peroneal nerve may be approach surgically. The common peroneal nerve may be identified by palpation as it wraps around the neck of the fibula, and an incision may be made immediately over the nerve. The fibers of the biceps femoris muscle may be separated to expose the common peroneal nerve, and the articular nerves springing from it. This approach may limit damage to the biceps to the most distal fibers of the muscle.

In some embodiments, a user may curatively or palliatively treat the knee by ablating the joint along one or more of the lines 2102, 2104, and 2106. For example, the knee may be treated at the dotted lines 2102, 2104, and/or 2106 to treat dysplasia, osteoarthritis, pain, or other conditions.

### Quadruped Elbow

Quadruped elbow joint pain is a common issues due to animal having a majority of its weight on the forelimbs, which is much different than with humans. In addition to the extra load and forces put on the elbow joints quadrupeds (canines in particular) suffer from dysplasia. Fragmented coronoid process (FCP), ununited anconeal process (UAP), osteochondritis dessicans (OCD) of the medial humeral condyle, ununited medial epicondyle (UME), and elbow joint incongruity have all been described as components of elbow dysplasia. In elbow dysplasia, the forces concentrated on specific areas of the joint will not only result in osteoarthritis (as happens with hip dysplasia) but also in discrete pathological entities like fractures within the joint that may need to be managed separately and alongside the osteoarthritis. Until recently it was believed that these disease entities were the manifestations of different elbow pathology but as they were commonly seen in combination with each other and they all resulted in elbow osteoarthritis, the term elbow dysplasia was used as an umbrella term for all of them. We now believe that these disease entities are the manifestation of the same pathology, which is the abnormal conformation of the joint and therefore we often see them in combination with each other. Although we still use the term elbow dysplasia to describe them, this term is now mostly used to describe the main pathology and not as an umbrella term for different joint disease entities.

In addition to predisposition to congenital abnormalities, quadrupeds also have distinct differences that differentiate them from humans. For example, the canine humeral head is less rounded compared with the human head, to assist with weight bearing. Distally, there is an olecranon fossa and supratrochlear foramen for the secure positioning of the protruding anconeal process of the ulna for more stability in weight bearing. The radius is the medial forearm bone and is the main weight-bearing bone of the antebrachium distally. The proximal surface of the radius articulates with the humeral capitulum, which is not as prominent as in the human. The canine distal radius has distinct facets for articulation with carpal bones, providing stability in weight bearing. The ulna is the lateral forearm bone and has a very prominent olecranon process, which allows secure attachment for the large triceps brachii muscle, needed as an antigravity muscle for weight bearing in dogs. The ulna is the longest bone of the canine body. It articulates distally with the ulnar carpal and accessory carpal bones by two distal facets and does not have an articular disk. The dog has an anconeal process, which is near the attachment site of the anconeus muscle. The anconeal process is needed for stability in weight bearing. The radius and ulna are equivalent to the bones of the human lower arm but, unlike the human, they are fused together to prevent the horse's foreleg from twisting. The ulna is very small except for the olecranon process, which forms part of the elbow. The elbow is a ginglymus joint between the humerus, radius and ulna, and allows movement in one direction only.

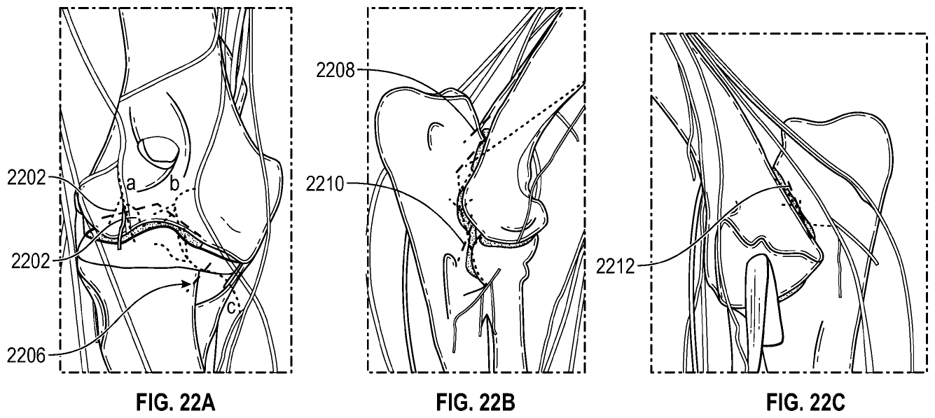

FIG. 22A illustrates an example of placement of a probe via a cranial view of an elbow. The treatment device (e.g., the device 600, 800, 900, 1000, 1100, 1200, 1700 and any probe herein) may be used to modify nerves associated with the elbow. A user may modify one or more of the branches of the radial nerve by disposing the first electrode and the second electrode of the treatment device along the dotted line 2202 and generating a bipolar radiofrequency between the first and second electrode. Similarly, one or more branches of the musculoskeletal nerve may be modified by disposing the first electrode and the second electrode of the treatment device along the dotted line 2204 and generating a bipolar radiofrequency between the first and second electrode. One or more branches of the median nerve may be modified by disposing the first electrode and the second electrode of the treatment device along the dotted line 2206 and generating a bipolar radiofrequency between the first and second electrode. In some embodiments, the treatment device may treat multiple areas of the elbow at once, such as a first area along the dotted line 2202 and a second area long the dotted line 2204. In some embodiments, the treatment device may be configured to ablate a first area along the dotted line 2102 and a second area along the dotted line 2104 in a single pulse.

In several embodiments, the devices and methods described herein are specifically designed to treat quadrupeds. Alternate embodiments are designed to accommodate ablation or other forms of neuromodulation in humans. Quadruped elbow sensory innervation and location of joint pain are different due to differences normal weight bearing of the joint, anatomical differences, and diseases not common in humans, and several embodiments herein are designed to uniquely accommodate such differences.

The radial (deep branch) nerve has innervation in the cranio-lateral region. As the radial nerve crosses the flexor surface of the elbow joint it sends articular branch medially into the craniolateral portion of joint capsule. This nerve contributed to innervation of the craniolateral aspect of the articular capsule in dogs by 1(70 %) or 2(20 %) branches given off at the flexor side of the elbow joint. Target sides of articular branches of radial nerve were proximal of the annular ligament in several instances, and distal to annular ligament in other instances. Deep Branches of radial n. can be located obliquely midline to lateral on the cranial of elbow joint and gave off several branches to articular capsule. These branches were found to penetrate the articular capsule from the craniolateral aspect. Thus, in several embodiments, a user may modify one or more of the branches of the radial nerve a by disposing the first electrode and the second electrode of the treatment device along the radial nerve and generating a bipolar radiofrequency between the first and second electrode. In some embodiments, the radial nerve may be approached surgically. In some embodiments the radial nerve may be approach surgically. The radial nerve may be identified by palpation as it wraps around the humerus, radius, and/or ulna, and an incision may be made immediately over the nerve. The fibers of the overlying muscles may be separated to expose the radial nerve, and the articular nerves springing from it. This approach may limit damage to the muscles around the elbow. In some embodiments, a user may curatively or palliatively treat the elbow by ablating the joint along one or more of lines of treatment. For example, the elbow may be treated to treat dysplasia, osteoarthritis, pain, or other conditions.

The median nerve has innervation in the medial/cranio-medial region. The median nerve crosses the flexor surface of the elbow joint cranial to the medial epicondyle. Small articular branch splits off and innervates medial aspect of joint capsule. The median nerve passes deep to the pronator teres and enters the large caudal group of flexor muscles located in the antebrachium. The median nerve gives muscular branches to the pronator teres, pronator quadratus, flexor carpi radialis, and flexor digitorum superficialis and the radial head of the flexor digitorum profundus. It also sends axons to the deep part of the humeral head of the flexor digitorum profundus and a small articular branch to the medial aspect of the elbow joint. Articular branches terminate near the middle of the proximodistal distance of the cranial aspect of articular capsule. This area is mediodistal to the site of entry of articular branch of musculocutaneous nerve to the capsule. Thus, in several embodiments a user may modify one or more of the branches of the median nerve a by disposing the first electrode and the second electrode of the treatment device along the median nerve and generating a bipolar radiofrequency between the first and second electrode. In some embodiments, the median nerve may be approached surgically. In some embodiments the median nerve may be approach surgically. The median nerve may be identified by palpation as it wraps around the humerus, radius, and/or ulna, and an incision may be made immediately over the nerve. The fibers of the overlying muscles may be separated to expose the median nerve, and the articular nerves springing from it. This approach may limit damage to the muscles around the elbow. In some embodiments, a user may curatively or palliatively treat the elbow by ablating the joint along one or more of lines of treatment. For example, the elbow may be treated to treat dysplasia, osteoarthritis, pain, or other conditions.

The ulnar nerve has innervation in the medial region. In dogs, ulnar nerve have an articular branch to the articular capsule of elbow joint during its course at the medial of the elbow joint between medial epicondyle and olecranon, and between the ulnar head of the flexor carpi ulnaris muscle and flexordigitalis superficialis muscle. Articular branches are localized at the medial part of the articular capsule. Thus, in several embodiments, a user may modify one or more of the branches of the ulnar nerve a by disposing the first electrode and the second electrode of the treatment device along the ulnar nerve and generating a bipolar radiofrequency between the first and second electrode. In some embodiments, the ulnar nerve may be approached surgically. In some embodiments the ulnar nerve may be approach surgically. The ulnar nerve may be identified by palpation as it wraps around the humerus, radius, and/or ulna, and an incision may be made immediately over the nerve. The fibers of the overlying muscles may be separated to expose the ulnar nerve, and the articular nerves springing from it. This approach may limit damage to the muscles around the elbow. In some embodiments, a user may curatively or palliatively treat the elbow by ablating the joint along one or more of lines of treatment. For example, the elbow may be treated to treat dysplasia, osteoarthritis, pain, or other conditions.

The musculocutaneous nerve has innervation in the cranial region. The musculocutaneous nerve gives off articular branches to articular capsule in dogs. Articular branches running into articular capsule originated from either distal part of the muscular branch of the musculocutaneous nerve or initial part of the medial antebrachial cutaneous nerve. Articular branches were usually localized at the cranial side of the articular capsule, proximal to the insertion of biceps brachii muscle. Thus, in several embodiments, a user may modify one or more of the branches of the musculocutaneous nerve a by disposing the first electrode and the second electrode of the treatment device along the musculocutaneous nerve and generating a bipolar radiofrequency between the first and second electrode. In some embodiments, the musculocutaneous nerve may be approached surgically. In some embodiments the musculocutaneous nerve may be approach surgically. The musculocutaneous nerve may be identified by palpation as it wraps around the humerus, radius, and/or ulna, and an incision may be made immediately over the nerve. The fibers of the overlying muscles may be separated to expose the musculocutaneous nerve, and the articular nerves springing from it. This approach may limit damage to the muscles around the elbow. In some embodiments, a user may curatively or palliatively treat the elbow by ablating the joint along one or more of lines of treatment. For example, the elbow may be treated to treat dysplasia, osteoarthritis, pain, or other conditions. FIG. 22B illustrates an example of placement of a probe via a lateral view of an elbow. The treatment device (e.g., the device 600, 800, 900, 1000, 1100, 1200, 1700) may be used to modify nerves associated with the elbow. A user may modify one or more of the branches of the radial, median, ulnar, and/or musculocutaneous nerves by disposing the first electrode and the second electrode of the treatment device along the dotted line 2208 and/or the dotted line 2010 and generating a bipolar radiofrequency between the first and second electrode. Other forms of neuromodulation (such as ablation) may also be used (including but not limited to heat, cryo, ultrasound, microwave, etc.).

FIG. 22C illustrates an example of placement of a probe via a medial view of an elbow. The treatment device (e.g., the device 600, 800, 900, 1000, 1100, 1200, 1700) may be used to modify nerves associated with the elbow. A user may modify one or more of the branches of the radial, median, ulnar, and/or musculocutaneous nerves by disposing the first electrode and the second electrode of the treatment device along the dotted line 2212 and generating a bipolar radiofrequency between the first and second electrode. Other forms of neuromodulation (such as ablation) may also be used (including but not limited to heat, cryo, ultrasound, microwave, etc.).

In some embodiments, a user may curatively or palliatively treat the elbow by ablating the joint along one or more of the lines 2202, 2204, 2206, 2208, 2210, and 2212. For example, the elbow may be treated at the dotted lines 2202, 2204, 2206, 2208, 2210, and/or 2212 to treat dysplasia, osteoarthritis, pain, or other conditions.

### Quadruped Shoulder

Quadrupeds are much different than humans in their usage of their forelimbs. For example, quadrupeds do not have clavicles. Unlike humans, the quadruped forelimbs have the most amount of weight on them (up to 62%) and endure tremendous amounts of force and strain. There are significant anatomical and morphological differences to accommodate for these differences. Additionally, the onset of joint disease and pain can occur much earlier and more often. The shoulder of quadrupeds is much less mobile than in humans. It functions like a roller joint vs. a true ball and socket joint. Thus, several embodiments described herein are designed to accommodate the quadruped shoulder anatomy.

The human shoulder joint is highly mobile, typically allowing for abduction of 90°, adduction of 20°, anteversion of 90°, and retroversion of 30°. Normal values of internal and external rotation of the humerus are each 70°. However, the large range of movement of the human shoulder is increased further by contribution from the scapulothoracal, sternoclavicular, and the acromioclavicular joints, leading to abduction of 180°and anteflexion of approximately 170°. In contrast, the range of motion of the shoulder joint in quadrupeds is limited because of the adjacent muscles and tendons, which lead the joint to function as a roller joint. Extension and flexion in carnivores typically is greater than 120°, with external rotation of up to 45°, but internal rotation is usually less than 35°. In several embodiments, human shoulder anatomy is treated.

Additional differences between humans and canines include the joint ligaments, tendons of the shoulder joint, and lack of rotator cuff. The structural and biomechanical characteristics of the joints of quadrupedal animals raise the question of their appropriateness for shoulder research. The surface relation of the glenoid cavity to humeral head is about 1:3 in dogs. In humans the humeral head is nearly 4 times larger than the glenoid cavity.

One of the most important differences is that the innervation of the joint is different from the human. Quadruped innervation of the shoulder includes Musculocutaneous, Suprascapular, and Axillary nerves. Human shoulder innervation includes Lateral Pectoral, Lower subscapular, Axillary, and Suprascapular nerves. These differences needs novel approaches and treatments for quadruped shoulder joint pain.

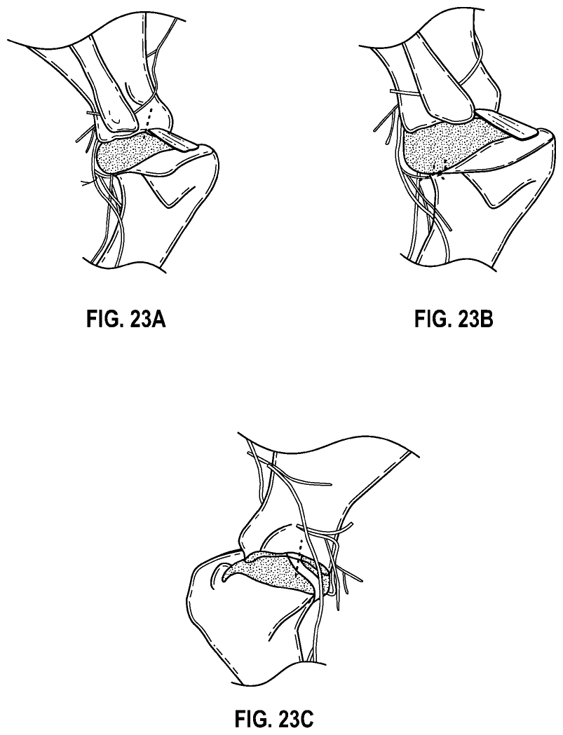

FIG. 23A illustrates an example of placement of a probe via a lateral view of a shoulder. The treatment device (e.g., the device 600, 800, 900, 1000, 1100, 1200, 1700) may be used to modify nerves associated with the shoulder. A user may modify one or more of the branches of the suprascapular nerve by disposing the first electrode and the second electrode of the treatment device along the dotted line 2302 and generating a bipolar radiofrequency between the first and second electrode. Other forms of neuromodulation (such as ablation) may also be used (including but not limited to heat, cryo, ultrasound, microwave, etc.).

FIG. 23B illustrates an example of placement of a probe via a lateral view of a shoulder. The treatment device (e.g., the device 600, 800, 900, 1000, 1100, 1200, 1700) may be used to modify nerves associated with the shoulder. A user may modify one or more of the branches of the axillary nerve by disposing the first electrode and the second electrode of the treatment device along the dotted line 2304 and generating a bipolar radiofrequency between the first and second electrode. Other forms of neuromodulation (such as ablation) may also be used (including but not limited to heat, cryo, ultrasound, microwave, etc.).

FIG. 23C illustrates an example of placement of a probe via a lateral view of a shoulder. The treatment device (e.g., the device 600, 800, 900, 1000, 1100, 1200, 1700) may be used to modify nerves associated with the shoulder. A user may modify one or more of the branches of the musculocutaneous nerve by disposing the first electrode and the second electrode of the treatment device along the dotted line 2306 and generating a bipolar radiofrequency between the first and second electrode. Other forms of neuromodulation (such as ablation) may also be used (including but not limited to heat, cryo, ultrasound, microwave, etc.).

In some embodiments, a user may curatively or palliatively treat the shoulder by ablating the joint along one or more of the lines 2302, 2304, and 2306. For example, the shoulder may be treated at the dotted lines 2302, 2304, and/or 2306 to treat osteoarthritis, pain, or other conditions.

### Quadruped Carpal Joint

The metacarpophalangeal (MCP) joint is the most common joint for spontaneous osteoarthritis in the racehorse, followed by the carpal joints. Both joints have close fitting articular surfaces that can quickly develop linear erosions and wear lines in association with osteochondral fragmentation. The carpal in a dog is roughly equivalent of a wrist for humans, except quadrupeds put pressure on it 24/7 when they are standing. A dog has almost its entire weight on its carpals (as well as its hocks in the rear end) so any carpal pain in dogs can be extremely painful. In several embodiments, carpals are treated.

Degeneration of the carpal bones beneath the cartilage are associated with Subchondral Bone Disease. Most fractures that occur within the carpal joint are likely preceded by the death of cells in the third carpal bone. This degeneration of cells is believed to be caused by recurring trauma. Bone disease beneath the cartilage elsewhere in the carpus may also cause degeneration of carpal bones. Signs include lameness, reduced performance, and swelling of the joint. Depending on the location, degeneration can be diagnosed either by using x-rays or by surgically inspecting the inside of the joint with an endoscope or arthroscope). Treatment involves surgically removing any dead, damaged, or infected tissue with an arthroscope.

Torn Medial Palmar Intercarpal Ligament: this injury is usually diagnosed when there is an inflammation of the membranes of the carpal joint that does not respond to treatment. Bone chips may be present in the carpus, and the horse may be exceedingly lame. Diagnosis is made by surgical inspection of the joint using an endoscope. An endoscope is also used for standard treatment to remove the torn fibers. The outlook for recovery depends on the amount of tearing and the presence of underlying bone damage. Studies of the effect of angular limb deformities on future musculoskeletal problems show conflicting results as to impact on performance whereas other carpal conformational deformities (e.g., “offset knees” conformation) have been associated with an increased risk of injury. Osteochondrosis is an equine developmental bone disease at multiple predisposed sites as a result of endochondral ossification. Osteochondrosis of the carpus is rare; however, osteochondrosis dissecans of the third and fourth carpal bones is reported in foals with angular limb deformities, and subchondral cyst-like lesions (thought to be a manifestation of osteochondrosis) have been reported in the medial aspect of the distal radial epiphysis in juvenile horses. Osteochondromata are cartilaginous exostoses characterized by a radiolucent hyaline cartilage cap overlying endochondral bone that communicates with the medullary cavity of the parent bone. Osteochondromata arising from defects of the perichondrial ring of the distal radial physis can grow and protrude into the carpal canal, leading to deep digital flexor tendinitis, synovitis of the carpal sheath, and lameness that could affect current, and potentially future, performance. In several embodiments, the treatment device may be used to modify nerves associated with the carpal joint. In several embodiments, the treatment device may be used to treat osteochondromata by ablating the cartilaginous exostoses.

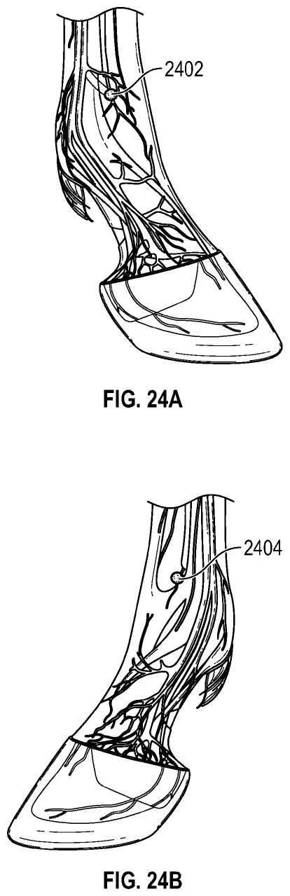

FIG. 24A illustrates an example of placement of a probe via a medial view of a metacarpophalangeal joint. The treatment device (e.g., the device 600, 800, 900, 1000, 1100, 1200, 1700) may be used to modify nerves associated with the metacarpophalangeal joint. A user may modify one or more of the branches of the medial palmar metacarpal nerve by disposing the first electrode and the second electrode of the treatment device at the point 2402 and generating a bipolar radiofrequency between the first and second electrode. Other forms of neuromodulation (such as ablation) may also be used (including but not limited to heat, cryo, ultrasound, microwave, etc.).

FIG. 24B illustrates an example of placement of a probe via a lateral view of a metacarpophalangeal joint. The treatment device (e.g., the device 600, 800, 900, 1000, 1100, 1200, 1700) may be used to modify nerves associated with the metacarpophalangeal joint. A user may modify one or more of the branches of the lateral palmar metacarpal nerve by disposing the first electrode and the second electrode of the treatment device at the point 2404 and generating a bipolar radiofrequency between the first and second electrode. Other forms of neuromodulation (such as ablation) may also be used (including but not limited to heat, cryo, ultrasound, microwave, etc.).

In some embodiments, a user may curatively or palliatively treat the metacarpophalangeal joint by ablating the joint at one or more of the first point 2402 and the second point 2404. For example, the metacarpophalangeal may be treated at the first point 2402 and/or the second point 2404 to treat osteoarthritis, synovitis, capsulitis, pain, or other conditions.

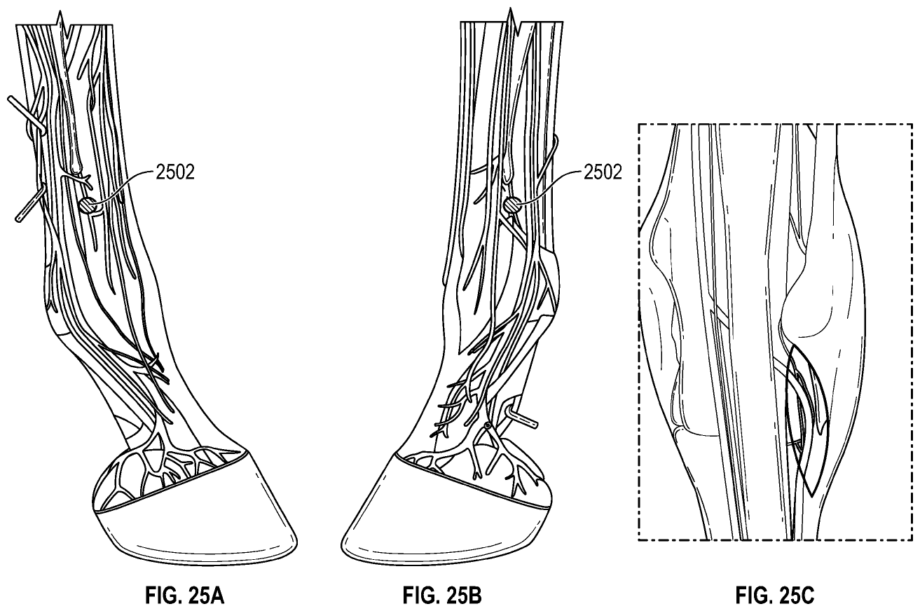

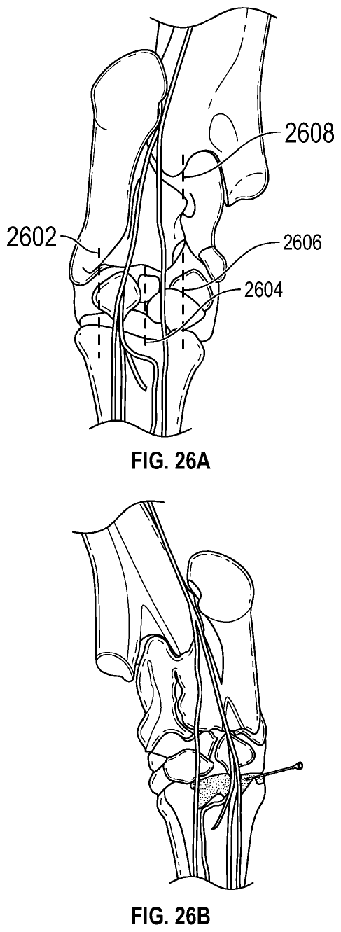

FIG. 25A illustrates an example of placement of a probe via a medial view of a metatarsophalangeal joint. The treatment device (e.g., the device 600, 800, 900, 1000, 1100, 1200, 1700) may be used to modify nerves associated with the metatarsophalangeal joint. A user may modify one or more of the branches of the medial palmar metatarsal nerve by disposing the first electrode and the second electrode of the treatment device at the point 2502 and generating a bipolar radiofrequency between the first and second electrode. Other forms of neuromodulation (such as ablation) may also be used (including but not limited to heat, cryo, ultrasound, microwave, etc.).

FIG. 25B illustrates an example of placement of a probe via a lateral view of a metatarsophalangeal joint. The treatment device (e.g., the device 600, 800, 900, 1000, 1100, 1200, 1700) may be used to modify nerves associated with the metatarsophalangeal joint. A user may modify one or more of the branches of the lateral palmar metatarsal nerve by disposing the first electrode and the second electrode of the treatment device at the point 2504 and generating a bipolar radiofrequency between the first and second electrode. Other forms of neuromodulation (such as ablation) may also be used (including but not limited to heat, cryo, ultrasound, microwave, etc.).

In some embodiments, a user may curatively or palliatively treat the metatarsophalangeal joint by ablating the joint at one or more of the first point 2502 and the second point 2504. For example, the metatarsophalangeal may be treated at the first point 2502 and the second point 2504 to treat osteoarthritis, synovitis, capsulitis, pain, or other conditions.

FIG. 25C illustrates an example incision for placement of a probe via a cranial view of a metatarsophalangeal joint.

### Quadruped Hock

Humans do not have a hock. A close similar structure would be the ankle. In equines, the hock is a complex joint and plays a major part in the generation of power to jump and gallop, as well as to sit in advanced dressage movements. Hocks are prone to both degeneration and injury. Probably the most common hock issue is arthritis; this can present subtlety as a small drop in performance, up to signs as obvious as moderate to severe lameness. It is rare to see hock arthritis other than in the lower two hock joints. One of the most common causes of lameness in horses is arthritis of the lower hock joints. Almost all hard-working horses over the age of 5 will have some pathology in these joints. There are many different treatments for hock arthritis including injecting the joints with cortisone and hyaluronic acid. Also, sometimes people ride horses while the horse is on anti-inflammatories like bute, and many of them are on oral joint supplements with glucosamine. However, some of these horses are so severely affected that many of these treatments are ineffective or at least do not last very long.

The next step in treatment is to arthrodese the joints, or destroy the joints so that the bones grow together. This decreases the pain in the area because if there is no longer a joint then there is no arthritis pain. In the past, destroying the joint was a surgical procedure accomplished with a laser or by drilling out the cartilage in the joint. In several embodiments described herein, neuromodulation, such as ablation, preserves joints, cartilage, and mobility.

For the hock, the lateral and medial plantar nerves lie plantar to their satellite vessels along the dorsal borders of the digital flexor tendons. These nerves supply the lateral, medial, and plantar structures of the metatarsus. A related issue near the hock is chronic suspensory disease of the ligament attachment to Cannon Bone. In certain cases, neurectomy of the deep branch of the lateral plantar nerve, which is closer to the area in the hock rather than the heel, is a viable option for horses with chronic suspensory disease at the origin or where the ligament attaches to the cannon bone. Pain or chronic lameness often remains even after proper treatment and rehabilitation due to enlargement of the ligament, causing a compartment-like syndrome and compression on the nerve. A very small branch of the lateral plantar nerve that supplies the suspensory origin is removed during the procedure. The main nerve supply to the foot and limb is not affected.

FIG. 26A illustrates an example of placement of a probe via a medial view of a hock. The treatment device (e.g., the device 600, 800, 900, 1000, 1100, 1200, 1700) may be used to modify nerves associated with the hock joint. A user may modify the medial plantar nerve and/or the lateral plantar nerve by placing the treatment device at along one or more of the first dotted line 2602, the second dotted line 2604, the third dotted line 2606, and the fourth dotted line 2608. In some embodiments, the treatment device may be adapted to modify tissue along two or more of the dotted lines. Once the treatment device is in position, a bipolar radiofrequency may be generated between the first and second electrode. Other forms of neuromodulation (such as ablation) may also be used (including but not limited to heat, cryo, ultrasound, microwave, etc.).

FIG. 26B illustrates an example of placement of a probe via a lateral view of a hock.

In some embodiments, a user may curatively or palliatively treat the hock by ablating the joint along one or more of one or more of the lines 2602, 2604, 2606, and 2608. For example, the hock may be treated along the lines 2602, 2604, 2606, and/or 2608 to treat osteoarthritis, chronic suspensory disease, pain, or other conditions.

### Quadruped Fetlock

The fetlock joint is unique to certain large animals (equines mostly and some large dogs) and is a very complex and high motion joint that is often subjected to huge forces and stresses during locomotion. Treatment for fetlock joint pain is usually conservative with injections (HA, steroids, etc.) being the most invasive (exception would be if there was a fracture). Arthrodesis of the joints is another option for severe cases. As the fetlock is a high motion joint, even relatively mild arthritis can adversely affect performance. Modulation of the following nerves and/or their branches that innervate the fetlock joint: Medial palmar metacarpal n., Lateral palmar metacarpal n., Medial palmar metatarsal n., Lateral palmar metatarsal n.

The fetlock joint is Metatarsophalangeal in both the forelimb and hindlimb. In the forelimb, nerves include Lateral palmar metacarpal nerve and the Medial palmar metacarpal nerve. Soon after receiving the branch of the ulnar, the lateral palmar nerve gives off a deep branch that innervates the interosseus and is continued by the medial and lateral palmar metacarpal nerves. These are deeply placed and run along the axial surfaces of the two splint bones where they are accompanied by equally thin arteries.

In the hindlimb, nerves include the Lateral palmar metatarsal nerve and the Medial palmar metatarsal nerve. The medial and lateral plantar metatarsal nerves arise from the deep branch of the lateral plantar nerve and distribute themselves as their counterparts in the forelimb. That is to say, they pass along the axial surfaces of the splint bones, innervate (part of) the fetlock joint and the skin on the dorsal surface of the proximal phalanx. The medial and lateral dorsalmetatarsal nerves are terminal branches of the deep peroneal nerve.

Osteoarthritis of the fetlock joint is common and can be insidious in onset and have no known causes. Known causes such as fragmentation, fracture, and luxation can ultimately lead to osteoarthritis and treatments are aimed at preventing such a process. Insidious osteoarthritis shows slow worsening of clinical signs including synovitis, capsulitis, reduced range of motion, and pain. In athletes such as racehorses, the accumulation of damage over time likely leads to the insidious onset of osteoarthritis. Progressive loss of articular cartilage can occur as can joint capsule fibrosis and limited range of motion. A wide spectrum of clinical signs can be apparent, and early signs include synovial effusion, reduced range of motion, and recurring lameness. However, it must be remembered that for disease processes that start within the subchondral bone, lameness may not be associated with any outward signs of disease in the fetlock joint. These cases require more in-depth diagnostic work-up, as mentioned before. As the severity of disease worsens, synovial effusion will continue to be apparent but will be compounded by joint capsule thickening, soft tissue swelling, reduced range of motion (both passively and actively during movement), and pain. Diagnosis of subtle injuries can be challenging and often require volumetric imaging to best characterize the pathologic process. The treatment of osteoarthritis depends on its stage of severity. Synovitis and capsulitis can be effectively treated through intraarticular medication. Mesenchymal stem cells are sometimes recommended at this stage to prevent worsening of articular cartilage damage.

For more advanced lesions, more continual therapy and strict management and oversight of exercise are often necessary to prolong the career and athletic ability of horses with fetlock osteoarthritis. Occasionally diagnostic arthroscopy may be useful to debride cartilage lesions although this method of treatment is controversial. Use of stem cells may be needed, although efficacy correlated with severity of osteoarthritis is unknown. In the most severe cases, fetlock arthrodesis may be needed to provide pain relief and prevent contralateral limb laminitis.

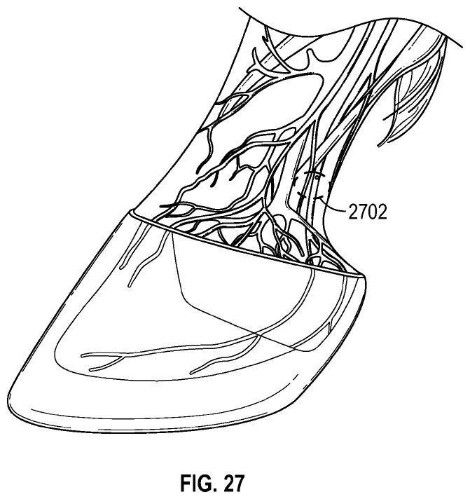

FIG. 27 illustrates an example of placement of a treatment device at the fetlock via a lateral view of a coffin joint. The treatment device (e.g., the device 600, 800, 900, 1000, 1100, 1200, 1700) may be used to modify nerves associated with the coffin joint. A user may modify one or more of the branches of the lateral palmar digital nerve by disposing the first electrode and the second electrode of the treatment device at the point 2702 and generating a bipolar radiofrequency between the first and second electrode. Other forms of neuromodulation (such as ablation) may also be used (including but not limited to heat, cryo, ultrasound, microwave, etc.). In some embodiments, a user may curatively or palliatively treat the coffin joint by ablating at the point 2702 to treat osteoarthritis, degeneration of the carpal bones, a torn medial palmar intercarpal ligament, pain, or other conditions.

Although described herein with respect mainly to canine and equine joints, the methods and systems described herein may be suitable for other joints and species. For example, hip pain in canines, equines, felines (including big cats such as lions and tigers), bovines, porcines, pachyderms, and other species maybe treated by modulating an articular branch nerve. Some bulls are not able to naturally mate due to hip joint pain inhibiting the bull from being able to mount the heifer, usually leaving artificial insemination and pharmaceuticals as the only options. Modulation as described herein can relieve the pain to allow natural insemination, and can avoid pharmaceuticals that might otherwise wind up in beef from the bull. For additional examples, stifle, elbow, and/or spine pain in canines, equines, felines, bovines, porcines, pachyderms, and other species maybe treated by modulating an articular branch nerve.

The foregoing description and examples has been set forth merely to illustrate the disclosure and are not intended as being limiting. Each of the disclosed aspects and examples of the present disclosure may be considered individually or in combination with other aspects, examples, and variations of the disclosure. In addition, unless otherwise specified, none of the steps of the methods of the present disclosure are confined to any particular order of performance. Modifications of the disclosed examples incorporating the spirit and substance of the disclosure may occur to persons skilled in the art and such modifications are within the scope of the present disclosure. Furthermore, all references cited herein are incorporated by reference in their entirety.

The features of the devices 600, 900, 1000, 1100, 1200 can be combined in some embodiments. Non-limiting examples include: a device comprising a movable ramp, a steerable stylet, and a veneered stylet; a device comprising a movable ramp and a steerable stylet; a device comprising a movable ramp and a veneered stylet; and a device comprising a steerable and veneered stylet, etc.

While the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various examples described and the appended claims. Further, the disclosure herein of any particular feature, aspect, method, property, characteristic, quality, attribute, element, or the like in connection with an example can be used in all other examples set forth herein. Any methods disclosed herein need not be performed in the order recited. Depending on the example, one or more acts, events, or functions of any of the algorithms, methods, or processes described herein can be performed in a different sequence, can be added, merged, or left out altogether (e.g., not all described acts or events are necessary for the practice of the algorithm). In some examples, acts or events can be performed concurrently, e.g., through multi-threaded processing, interrupt processing, or multiple processors or processor cores or on other parallel architectures, rather than sequentially. Further, no element, feature, block, or step, or group of elements, features, blocks, or steps, are necessary or indispensable to each example. Additionally, all possible combinations, subcombinations, and rearrangements of systems, methods, features, elements, modules, blocks, and so forth are within the scope of this disclosure. The use of sequential, or time-ordered language, such as “then,” “next,” “after,” “subsequently,” and the like, unless specifically stated otherwise, or otherwise understood within the context as used, is generally intended to facilitate the flow of the text and is not intended to limit the sequence of operations performed. Thus, some examples may be performed using the sequence of operations described herein, while other examples may be performed following a different sequence of operations.

Conditional language used herein, such as, among others, “can,” “might,” “may,” “e.g.,” and the like, unless specifically stated otherwise, or otherwise understood within the context as used, is generally intended to convey that some examples include, while other examples do not include, certain features, elements, and/or states. Thus, such conditional language is not generally intended to imply that features, elements, blocks, and/or states are in any way required for one or more examples or that one or more examples necessarily include logic for deciding, with or without author input or prompting, whether these features, elements and/or states are included or are to be performed in any particular example.

The methods disclosed herein may include certain actions taken by a practitioner; however, the methods can also include any third-party instruction of those actions, either expressly or by implication. For example, actions such as “positioning a catheter” include “instructing positioning of a catheter.”

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 4 mm” includes “4 mm.” Phrases preceded by a term such as “substantially” include the recited phrase and should be interpreted based on the circumstances (e.g., as much as reasonably possible under the circumstances). For example, “substantially linear” includes “linear.” Unless stated otherwise, all measurements are at standard conditions including temperature and pressure.

The phrase “at least one of” is intended to require at least one item from the subsequent listing, not one type of each item from each item in the subsequent listing. For example, “at least one of A, B, and C” can include A; B; C; A and B; A and C; B and C; or A, B, and C.

What is claimed is:

1. A method of ablating an equine or canine articular nerve branch in a shoulder or hip, the method comprising:
imaging an equine or canine joint capsule using at least one of ultrasound or fluoroscopy to identify anatomical landmarks in a shoulder or hip;
percutaneously inserting a treatment device to a first point determined by the anatomical landmarks until a distal end of the treatment device contacts a bone,
wherein the first point is disposed at one side selected from the group of: a medial side of the joint capsule and a lateral side of the joint capsule;
radially extending a stylet laterally out of a needle body of the treatment device along a surface of the bone at an angle in a range of 45°-135° and a length of 20-90 mm,
wherein extending the stylet comprises sliding the stylet along the bone to a second point,
wherein the needle body comprising a first electrode and the stylet comprising a second electrode;
imaging the joint capsule using the at least one of ultrasound or fluoroscopy to confirm a first location of the first electrode and a second location of the second electrode relative to the anatomical landmarks;
applying bipolar radiofrequency energy to the first electrode and the second electrode to ablate a first tissue between the first point and the second point, the first tissue including an articular nerve branch;
retracting the stylet into the needle body; and
retracting the treatment device out of a subject.

2. A minimally invasive method of ablating an articular nerve branch adjacent to a joint capsule to treat joint pain in a subject, the method comprising:
percutaneously inserting a treatment device to a first point until a distal end of the treatment device contacts a bone;
radially extending a stylet laterally out of a needle body of the treatment device along a surface of the bone at an angle in a range of 45°-135° and a length of 30-60 mm,
wherein extending the stylet comprises sliding the stylet along the bone to a second point,
wherein the needle body comprising a first electrode and the stylet comprising a second electrode;
applying bipolar radiofrequency energy to the first electrode and the second electrode to ablate first tissue between the first point and the second point, the first tissue including an articular branch nerve;
retracting the stylet into the needle body.

3. The method of claim 1 or 2, further comprising repeating the percutaneously inserting, extending, and applying to ablate a second tissue selected from the group consisting of: a sciatic nerve, a cranial gluteal nerve, a femoral nerve, and an obturator nerve.

4. The method of claim 1 or 2, further comprising repeating the percutaneously inserting, extending, and applying to ablate a third tissue selected from the group consisting of: a sciatic nerve, a cranial gluteal nerve, a femoral nerve, and an obturator nerve.

5. The method of claim 1 or 2, further comprising repeating the percutaneously inserting, extending, and applying to ablate a fourth tissue selected from the group consisting of: a sciatic nerve, a cranial gluteal nerve, a femoral nerve, and an obturator nerve.

6. The method of claim 1 or 2, wherein the subject is a quadruped.

7. The method of claim 1 or 2, wherein the joint capsule is in a hip, a shoulder, a knee, an elbow, a stifle, a fetlock, or a carpal.

8. The method of claim 1 or 2, further generating coordinates for a target treatment site as a function of a calculated distance from the anatomical landmarks.

9. The method of claim 1 or 2, wherein the subject has osteoarthritis or degenerative joint disease.

10. A minimally invasive method of ablating an articular nerve branch adjacent a joint quadrant to treat joint pain in a subject, the method comprising:
identifying an anatomical landmark;
percutaneously inserting a treatment device to a first point determined by the anatomical landmarks until a distal end of the treatment device contacts a bone,
radially extending a stylet laterally out of a needle body of the treatment device along a surface of the bone at an angle in a range of 45°-135°,
wherein extending the stylet comprises sliding the stylet along the bone to a second point,
wherein the needle body comprising a first electrode and the stylet comprising a second electrode;
wherein a first region between the first point and the second point corresponds to a first joint quadrant;
applying bipolar radiofrequency energy to the first electrode and the second electrode to ablate a first tissue between the first point and the second point, the first tissue including an articular nerve branch;
retracting the stylet into the needle body; and
retracting the treatment device out of the subject.

11. The method of claim 10, wherein extending the stylet further comprises:
sliding the stylet along the bone from the second point to a third point, wherein a second region between the second point and the third point corresponds to a second joint quadrant.

12. The method of claim 10, wherein extending the stylet further comprises:
sliding the stylet along the bone from the second point to a third point, wherein a second region between the second point and the third point corresponds to a third joint quadrant.

13. The method of claim 10, wherein extending the stylet further comprises:
sliding the stylet along the bone from the second point to a third point, wherein a second region between the second point and the third point corresponds to a fourth joint quadrant.

14. The method of any one of claims 10-13, wherein the subject is a quadruped.

15. The method of any one of claims 10-13, wherein the first joint quadrant is in a hip, a shoulder, a knee, an elbow, a stifle, a fetlock, or a carpal.

16. The method of any one of claims 10-13, further generating coordinates for a target treatment site as a function of a calculated distance from the anatomical landmarks.

17. The method of any one of claims 10-13, wherein the subject has osteoarthritis or degenerative joint disease.

18. A minimally invasive method of ablating an extended length of an articular nerve to increase a duration of joint pain relief, the method comprising:
percutaneously inserting a treatment device to a first point determined by an anatomical landmark;
radially extending a stylet laterally out of a needle body of the treatment device along a surface of a bone at an angle in a range of 45°-135°,
wherein extending the stylet comprises sliding the stylet along the bone to a second point lengthwise along an articular nerve,
wherein the needle body comprising a first electrode and the stylet comprising a second electrode;
imaging using at least one of ultrasound or fluoroscopy to confirm a location of the first electrode and the second electrode relative to the anatomical landmarks;
applying bipolar radiofrequency energy to the first electrode and the second electrode to ablate nerve tissue along a length of the nerve between the first point and the second point;
retracting the stylet into the needle body; and
retracting the treatment device out of a subject.

19. The method of claim 18, wherein the length of the nerve between the first point and the second point comprises a plurality of nociceptors.

20. A minimally invasive method of modulating one or more sensory nerves of a dorsal spinous process in a subject, the method comprising:
percutaneously inserting a treatment device to a first point determined by anatomical landmarks, wherein the first point is disposed at a first spinous process of a first vertebrae of the subject;
radially extending a stylet laterally out of a needle body of the treatment device along a surface of the first vertebrae at an angle in a range of 45°-135°,
wherein extending the stylet comprises sliding the stylet along a bone to a second point,
wherein the needle body comprising a first electrode and the stylet comprising a second electrode;
imaging using at least one of ultrasound or fluoroscopy to confirm a location of the first electrode and the second electrode relative to the anatomical landmarks;
applying bipolar radiofrequency energy to the first electrode and the second electrode to ablate a first tissue between the first point and the second point;
retracting the stylet into the needle body; and
retracting the treatment device out of the subject.

21. The method of claim 20, wherein the first tissue is one or more of: a dorsal ramus of the first vertebrae, a lateral dorsal ramus of the first vertebrae, a medial dorsal ramus of the first vertebrae.

22. The method of any one of claims 20-21, wherein the second point is one of: a transverse process of the first vertebrae or a transverse process of a second vertebrae.

23. A minimally invasive device for treating tissue, the device comprising:
a needle body comprising:
a tissue penetrating tip; and
an aperture in a side of the needle body;
a stylet configured to radially extend from the aperture of the needle body at an angle in a range of 45°-135°, wherein the stylet is configured for extending along a surface of a bone, the stylet comprising:
a first electrode, and
a tissue penetrating tip;
a second electrode disposed along the needle body between the tissue penetrating tip of the needle body and the stylet; and
a handle configured to be coupled to a tissue treatment system, the handle comprising a stylet mechanism configured to longitudinally move the stylet relative to the needle body.

24. The device of claim 23, wherein the needle body comprises a needle body temperature sensor.

25. The device of claim 23, wherein the stylet comprises a stylet temperature sensor,

26. The device of claim 23, wherein the stylet is configured to radially extend from the aperture of the needle body along a curved path.

27. The device of any one of claims 23-26, wherein the stylet mechanism comprises a knob configured to slide along a path.

28. The device of any one of claims 23-26, wherein the stylet mechanism comprises the handle being rotatable relative to the needle body.

29. The device of any one of claims 23-26, wherein the stylet mechanism comprises a rotatable knob.

30. The device of any one of claims 23-26, wherein the needle body comprises the first electrode.

31. The device of any one of claims 23-26, wherein the tissue penetrating tip of the needle body comprises the first electrode.

32. The device of any one of claims 23-26, wherein the tissue penetrating tip of the stylet comprises the first electrode.

33. The device of any one of claims 23-26, wherein the tissue penetrating tip of the stylet comprises the first electrode.

34. The device of any one of claims 23-26, wherein the stylet mechanism comprises indicia configured to inform a user about an extend of longitudinal movement of the stylet.

35. The device of any one of claims 23-26, wherein the stylet mechanism comprises detents configured to inform a user about an extend of longitudinal movement of the stylet.

36. The device of any one of claims 23-26, further comprising electronic circuitry configured to track use information.

37. The device of any one of claims 23 to 26, further comprising a longitudinally movable ramp, wherein a longitudinal position of the ramp affects a longitudinal position at which the stylet radially extends from the aperture of the needle body.

38. The device of any one of claims 23-26, wherein the handle comprises a ramp mechanism configured to longitudinally move the ramp relative to the needle body.

39. The device of any one of claims 23-26, wherein the stylet is steerable.

40. The device of any one of claims 23-26, wherein the stylet is steerable in one direction.

41. The device of any one of claims 23-26, wherein the stylet is steerable in two directions.

42. The device of claim 41, wherein the two directions are on one plane.

43. The device of any one of claims 23-26, wherein the stylet comprises one steering wire.

44. The device of any one of claims 23-26, wherein the stylet comprises two steering wires.

45. The device of any one of claims 23-26, wherein the stylet comprises a tube.

46. The device of claim 45, wherein the tube comprises a first plurality of kerfs on a first side of the tube and a second plurality of kerfs on a second side of the tube opposite the first side.

47. The device of claim 46, wherein at least one of a shape, size, or spacing of the first plurality of kerfs is different than at least one of a shape, size, or spacing of the second plurality of kerfs.

48. The device of claim 45, wherein a distal portion of the tube comprises a pattern and material bent radially inwardly to attach a steering wire between the material and an inner surface of the tube.

49. The device of any one of claims 23-26, wherein the stylet mechanism comprises a knob configured to slide along a path and to rotate relative to the needle body, wherein rotation of the knob is configured to steer the stylet.

50. The device of any one of claims 23-26, wherein the stylet comprises the first electrode and the second electrode longitudinally movable relative to each other.

51. The device of any one of claims 23-26, wherein the stylet comprises a first tube comprising the first electrode and a second tube comprising a second electrode, the second tube in telescoping arrangement with the first tube.

52. The device of any one of claims 23-26, wherein the stylet comprises a tube comprising a shape memory material.

53. A method of ablating an equine or canine articular nerve branch in the method comprising:
imaging a joint capsule using at least one of ultrasound or fluoroscopy to identify anatomical landmarks;
percutaneously inserting a treatment device to a first point determined by the anatomical landmarks until a distal end of the treatment device contacts a bone,
wherein the first point is disposed at one side selected from the group of: a medial side of the joint capsule and a lateral side of the joint capsule;
radially extending a stylet laterally out of a needle body of the treatment device at an angle in a range of 45°-135°,
wherein extending the stylet comprises sliding the stylet along the bone to a second point,
wherein the needle body comprising a first electrode and the stylet comprising a second electrode;
imaging the joint capsule using the at least one of ultrasound or fluoroscopy to confirm a first location of the first electrode and a second location of the second electrode relative to the anatomical landmarks;
applying bipolar radiofrequency energy to the first electrode and the second electrode to ablate a first tissue between the first point and the second point, the first tissue including an articular nerve branch;
retracting the stylet into the needle body; and
retracting the treatment device out of a subject.

54. A minimally invasive method of ablating an articular nerve branch adjacent to a joint capsule to treat joint pain in a subject, the method comprising:
percutaneously inserting a treatment device to a first point until a distal end of the treatment device contacts a bone;
radially extending a stylet laterally out of a needle body of the treatment device at an angle in a range of 45°-135°,
wherein extending the stylet comprises sliding the stylet along the bone to a second point,
wherein the needle body comprising a first electrode and the stylet comprising a second electrode;
applying bipolar radiofrequency energy to the first electrode and the second electrode to ablate first tissue between the first point and the second point, the first tissue including an articular branch nerve;
retracting the stylet into the needle body.

55. A treatment system having one or more of the features described in the foregoing description.

56. A tissue treatment system having one or more of the features described in the foregoing description.

57. A method of ablating an articulator nerve branch having one or more of the features described in the foregoing description.
